(19) United States
(12) Patent Application Publication
(10) Pub. No.: US 2005/0107374 A1
(43) Pub. Date: May 19, 2005

(54) Substituted heterocyclic compounds and methods of use

(75) Inventors: Daniel Elbaum, Newton, MA (US); Matthew W. Martin, Cambridge, MA (US); Joseph J. Nunes, Andover, MA (US)

Correspondence Address: AMGEN INC.;U.S. Patent Operations/GPR, Dept. 4300, M/S 27-4-A, One Amgen Center Drive, Thousand Oaks, CA, 91320-1799, US

(73) Assignee: Amgen Inc.

(21) Appl. No.: 10/969,826

(22) Filed: Oct. 20, 2004

Related U.S. Application Data
(60) Provisional application No. 60/513,234, filed on Oct. 21, 2003.

(51) Int. Cl. C07D 473/12; A61K 31/541; A61K 31/5377; A61K 31/52

(52) U.S. Cl. 514/227.8; 514/234.5; 514/241; 514/263.21; 514/263.2; 544/60; 544/114; 544/209; 544/277

(57) ABSTRACT

The present invention relates to hydroxybenzimidazole pyrimidines or pyridines or pharmaceutically-acceptable salts thereof. Also included is a method of treatment of inflammation, inhibition of T cell activation and proliferation, arthritis, rheumatoid arthritis, psoriatic arthritis, osteoarthritis, organ transplant, acute transplant or heterograft or homograft rejection, transplantation tolerance induction, ischemic or reperfusion injury, myocardial infarction, stroke, multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea, atopic dermatitis, colon carcinoma or thymoma in a mammal comprising administering a therapeutically-effective amount a compound as described above.

This application claims the benefit of U.S. Provisional Application No. 60/513,234 filed Oct. 21, 2003, which is hereby incorporated by reference.

### BACKGROUND OF THE INVENTION

T cells play a pivotal role in the regulation of immune responses and are important for establishing immunity to pathogens. In addition, T cells are often activated during inflammatory autoimmune diseases, such as rheumatoid arthritis, inflammatory bowel disease, type I diabetes, multiple sclerosis, Sjogren's disease, myasthenia gravis, psoriasis, and lupus. T cell activation is also an important component of transplant rejection, allergic reactions, and asthma.

T cells are activated by specific antigens through the T cell receptor (TCR) which is expressed on the cell surface. This activation triggers a series of intracellular signaling cascades mediated by enzymes expressed within the cell (Kane, L P et al. Current Opinion in Immunol. 200, 12, 242). These cascades lead to gene regulation events that result in the production of cytokines, like interleukin-2 (IL-2). IL-2 is a critical cytokine in T cell activation, leading to proliferation and amplification of specific immune responses.

One class of enzymes shown to be important in signal transduction are the kinases. There are eight members of the Src-family of tyrosine kinases: Lck, Fyn(B), Fyn(T), Lyn, Src, Yes, Hck, Fgr and Blklck (for review see: Bolen, J B, and Brugge, J S Annu. Rev. Immunol 1997, 15, 371). Gene disruption studies suggest that inhibition of some members of the src family of kinases would potentially lead to therapeutic benefit. Src(−/−) mice have abnormalities in bone remodeling or osteopetrosis (Soriano, P. Cell 1991, 64, 693), suggesting that inhibition of this kinase might be useful in diseases of bone resorption, such as osteoporosis. Lck(−/−) mice have defects in T cell maturation and activation (Anderson, S J et al. Adv. Immunol. 1994, 56, 151), suggesting that inhibition of this kinase might be useful in diseases of T cell mediated inflammation. In addition, human patients have been identified with mutations effecting Lck kinase activity (Goldman, F D et al. J. Clin. Invest.1998, 102, 421). These patients suffer from a severe combined immunodeficiency disorder (SCID).

Without wishing to imply that the compounds disclosed in the present invention possess pharmacological activity only by virtue of an effect on a single biological process, it is believed that the compounds modulate T cell activation by way of inhibition of one or more of the multiple protein tyrosine kinases involved in early signal transduction steps leading to T cell activation, for example by way of inhibition of Lck kinase.

Src-family kinases are also important for signaling downstream of other immune cell receptors. Fyn, like Lck, is involved in TCR signaling in T cells (Appleby, M W et al. Cell 1992, 70, 751). Hck and Fgr are involved in Fcγ receptor signaling leading to neutrophil activation (Vicentini, L. et al. J. Immunol. 2002, 168, 6446). Lyn and Src also participate in Fcγ receptor signaling leading to release of histamine and other allergic mediators (Turner, H. and Kinet, J-P Nature 1999, 402, B24). These findings suggest that Src family kinase inhibitors may be useful in treating allergic diseases and asthma.

Src kinases have also been found to be activated in tumors including sarcoma, melanoma, breast, and colon cancers suggesting that Src kinase inhibitors may be useful anti-cancer agents (Abram, C L and Courtneidge, S A Exp. Cell Res. 2000, 254, 1).

Src kinase inhibitors have also been reported to be effective in an animal model of cerebral ischemia (R. Paul et al. Nature Medicine 2001, 7, 222), suggesting that Src kinase inhibitors may be effective at limiting brain damage following stroke.

Several groups have published on inhibitors of Src family kinase and the activities of these inhibitors in various in vitro and in vivo biological systems. These include the 2-phenylamino-imidazo[4,5-h]isoquinolin-9-ones (Snow, R J et al. J. Med. Chem. 2002, 45, 3394), the pyrazolo[3,4-d]pyrimidines (Burchat, A F et al. Bioorganic and Med. Chem. Letters 2002, 12, 1687. Hanke, J H et al. J. Biol. Chem. 1996, 271, 695), the pyrrolo[2,3-d]pyrimidines (Altmann, E et al. Bioorganic and Med. Chem. Letters 2001, 11, 853), the anilinoquinazolines (Wang, Y D et al. Bioorganic and Med. Chem. Letters 2000, 10, 2477), and the imidazoquinoxalines (Chen, P. et al. Bioorganic and Med. Chem. Letters 2002, 12, 3153).

### BRIEF DESCRIPTION OF THE INVENTION

The compounds of the present invention inhibit protein tyrosine kinases, especially Src-family kinases such as Lck, Fyn(B), Fyn(T), Lyn, Src, Yes, Hck, Fgr and Blk, and are thus useful in the treatment, including prevention and therapy, of protein tyrosine kinase-associated disorders such as immunologic disorders. “Protein tyrosine kinase-associated disorders” are those disorders which result from aberrant tyrosine kinase activity, and/or which are alleviated by the inhibition of one or more of these enzymes. For example, Lck inhibitors are of value in the treatment of a number of such disorders (for example, the treatment of autoimmune diseases), as Lck inhibition blocks T cell activation. The treatment of T cell mediated diseases, including inhibition of T cell activation and proliferation, is a preferred embodiment of the present invention. Compounds of the present invention which selectively block T cell activation and proliferation are preferred. Also, compounds of the present invention which may block the activation of endothelial cell protein tyrosine kinase by oxidative stress, thereby limiting surface expression of adhesion molecules that induce neutrophil binding, and which can inhibit protein tyrosine kinase necessary for neutrophil activation would be useful, for example, in the treatment of ischemia and reperfusion injury.

The present invention also provides methods for the treatment of protein tyrosine kinase-associated disorders, comprising the step of administering to a subject in need thereof at least one compound of the formula I in an amount effective therefor. Other therapeutic agents such as those described below may be employed with the inventive compounds in the present methods. In the methods of the present invention, such other therapeutic agent(s) may be administered prior to, simultaneously with or following the administration of the compound(s) of the present invention.

Use of the compound(s) of the present invention in treating protein tyrosine kinase-associated disorders is exemplified by, but is not limited to, treating a range of disorders such as: arthritis (such as rheumatoid arthritis, psoriatic arthritis or osteoarthritis); transplant (such as organ transplant, acute transplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; multiple sclerosis; inflammatory bowel disease, including ulcerative colitis and Crohn's disease; lupus (systemic lupus erythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers where Lck or other Src-family kinases such as Src are activated or overexpressed, such as colon carcinoma and thymoma, or cancers where Src-family kinase activity facilitates tumor growth or survival; glomerulonephritis, serum sickness; uticaria; allergic diseases such as respiratory allergies (asthma, hayfever, allergic rhinitis) or skin allergies; scleracielma; mycosis fungoides; acute inflammatory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea. The present invention also provides for a method for treating the aforementioned disorders such as atopic dermatitis by administration of a therapeutically effective amount of a compound of the present invention, which is an inhibitor of protein tyrosine kinase, to a patient in need of such treatment.

Src-family kinases other than Lck, such as Hck and Fgr, are important in the Fcγ receptor induced respiratory burst of neutrophils as well as the Fcγ receptor responses of monocytes and macrophages. The compounds of the present invention may inhibit the Fcγ induced respiratory burst response in neutrophils, and may also inhibit the Fcγ dependent production of TNFα. The ability to inhibit Fcγ receptor dependent neutrophil, monocyte and macrophage responses would result in additional anti-inflammatory activity for the present compounds in additton to their effects on T cells. This activity would be especially of value, for example, in the treatment of inflammatory diseases, such as arthritis or inflammatory bowel disease. The present compounds may also be of value for the treatment of autoimmune glomerulonephritis and other instances of glomerulonephritis induced by deposition of immune complexes in the kidney that trigger Fcγ receptor responses and which can lead to kidney damage.

In addition, certain Src family kinases, such as Lyn and Fyn(B), may be important in the Fcε receptor induced degranulation of mast cells and basophils that plays an important role in asthma, allergic rhinitis, and other allergic disease. Fcε receptors are stimulated by IgE-antigen complexes. The compounds of the present invention may inhibit the Fcε induced degranulation responses. The ability to inhibit Fcε receptor dependent mast cell and basophil responses may result in additional anti-inflammatory activity for the present compounds beyond their effect on T cells.

The combined activity of the present compounds towards monocytes, macrophages, T cells, etc. may prove to be a valuable tool in the treatment of any of the aforementioned disorders.

In a particular embodiment, the compounds of the present invention are useful for the treatment of the aforementioned exemplary disorders irrespective of their etiology, for example, for the treatment of rheumatoid arthritis, transplant rejection, multiple sclerosis, inflammatory bowel disease, lupus, graft v. host disease, T cell mediated hypersensitivity disease, psoriasis, Hashimoto's thyroiditis, Guillain-Barre syndrome, cancer, contact dermatitis, allergic disease such as allergic rhinitis, asthma, ischemic or reperfusion injury, or atopic dermatitis whether or not associated with PTK.

The compounds of the invention are represented by the following general structure: 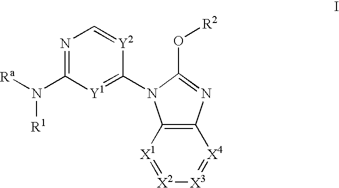
wherein X¹, X², X³, X⁴, Y¹, Y², R¹, R^{a} and R² are defined herein below.

The foregoing merely summarizes certain aspects of the invention and is not intended, nor should it be construed, as limiting the invention in any way. All patents and other publications recited herein are hereby incorporated by reference in their entirety.

### DETAILED DESCRIPTION OF THE INVENTION

In accordance with the present invention, one aspect of the invention relates to a compound of Formula I 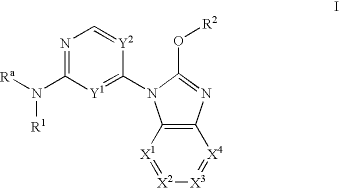
or a pharmaceutically-acceptable salt thereof, wherein
X¹ is N or C(R^{3a}); X² is N or C(R^{3b}); X³ is N or C(R^{3c}); X⁴ is N or C(R^{3d});
Y¹ is N or CH; Y² is N or CH;
R¹ is selected from —R¹¹, —R¹¹—R¹², —R¹¹—R¹⁴, —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴ and —R¹¹—R¹²—R¹³—R¹⁴, and of which is substituted by 0, 1, 2, 3 or 4 substituents independently selected from R^{c};
alternatively R¹ and R^{a} taken together with the nitrogen to which they are attached form a 5- or 6-membered heterocyclic ring having 0, 1 or 2 additional heteroatoms selected from N, O and S, which heterocyclic ring is substituted by 0, 1, 2, 3 or 4 substituents independently selected from R^{c};
R² is selected from —R²¹, —R²¹—R²², —R²¹—R²⁴, —R²²—R²⁴, —R²¹—R²²—R²⁴, —R²¹—R²³—R²⁴, —R²²—R²³—R²⁴, —R²¹—R²³—R²²—R²⁴ and —R²¹—R²²—R²³—R²⁴, any of which is substituted by 0, 1, 2, 3 or 4 substituents independently selected from R^{c};
R^{3a} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3a} is independently in each instance selected from R^{c};
R^{3b} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3b} is independently in each instance selected from R^{c};
R^{3c} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3c} is independently in each instance selected from R^{c};
R^{3d} is independently in each instance, selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3d} is independently in each instance selected from R^{c};
R¹¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R¹² is independently at each instance C₁₋₈alkyl;
R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R¹⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²² is independently at each instance C₁₋₈alkyl;
R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R²⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³² is independently at each instance C₁₋₈alkyl;
R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R³⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R^{a} is independently at each instance H or R^{b};
R^{b} is independently at each instance C₁₋₈alkyl, phenyl or benzyl; and
R^{c} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any of the above or below embodiments, X¹ is C(R^{3a}); X² is C(R^{3b}); X³ is C(R^{3c}); X⁴ is C(R^{3d}).

In another embodiment, in conjunction with any of the above or below embodiments, X¹ is N; X² is C(R^{3b}); X³ is C(R^{3c}); X⁴ is C(R^{3d}).

In another embodiment, in conjunction with any of the above or below embodiments, X¹ is C(R^{3a}); X² is N; X³ is C(R^{3c}); X⁴ is C(R^{3d}).

In another embodiment, in conjunction with any of the above or below embodiments, X¹ is C(R^{3a}); X² is C(R^{3b}); X³ is N; X⁴ is C(R^{3d}).

In another embodiment, in conjunction with any of the above or below embodiments, X¹ is C(R^{3a}); X² is C(R^{3b}); X³ is C(R^{3c}); X⁴ is N.

In another embodiment, in conjunction with any of the above or below embodiments, any one of X¹, X², X³ and X⁴ are N.

In another embodiment, in conjunction with any of the above or below embodiments, any two of X¹, X², X³ and X⁴ are N.

In another embodiment, in conjunction with any of the above or below embodiments, Y¹ is CH and Y² is N.

In another embodiment, in conjunction with any of the above or below embodiments, Y¹ is N and Y² is CH.

In another embodiment, in conjunction with any of the above or below embodiments, Y¹ is N and Y² is N.

In another embodiment, in conjunction with any of the above or below embodiments, Y¹ is CH and Y² is CH.

Embodiment A: In another embodiment, in conjunction with any of the above or below embodiments, R¹ is selected from —R¹¹, —R¹¹—R¹⁴, —R¹¹—R¹², —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴ and —R¹¹—R¹²—R¹³—R¹⁴, any of which is substituted by 0, 1, 2, 3 or 4 substituents independently selected from R^{c}.

In another embodiment, in conjunction with any of the above or below embodiments, R¹ is selected from —R¹¹, —R¹¹—R¹⁴, —R¹¹—R¹², —R¹¹—R¹²—R¹⁴, —R¹¹—R¹³—R¹⁴, —R¹¹—R¹³—R¹²—R¹⁴ and —R¹¹—R¹²—R¹³—R¹⁴, any of which is substituted by 0, 1, 2, 3 or 4 substituents independently selected from R^{c}.

In another embodiment, in conjunction with any of the above or below embodiments, R¹ is R¹¹, which is substituted by 0, 1, 2, 3 or 4 substituents independently selected from R^{c}.

Embodiment B: In another embodiment, in conjunction with any of the above or below embodiments, R¹ is —R¹¹—R¹⁴, which is substituted by 0, 1, 2, 3 or 4 substituents independently selected from R^{c}.

Embodiment C: In another embodiment, in conjunction with any of the above or below embodiments, R¹ is —R¹¹—R¹², which is substituted by 0, 1, 2, 3 or 4 substituents independently selected from R^{c}.

In another embodiment, in conjunction with any of the above or below embodiments, R¹ is —R¹¹—R¹²—R¹⁴, which is substituted by 0, 1, 2, 3 or 4 substituents independently selected from R^{c}.

In another embodiment, in conjunction with any of the above or below embodiments, R¹ is —R¹¹—R¹³—R¹⁴, which is substituted by 0, 1, 2, 3 or 4 substituents independently selected from R^{c}.

Embodiment D: In another embodiment, in conjunction with any of the above or below embodiments, R¹ is —R¹¹—R¹³—R¹²—R¹⁴, which is substituted by 0, 1, 2, 3 or 4 substituents independently selected from R^{c}.

In another embodiment, in conjunction with any of the above or below embodiments, R¹ is —R¹¹—R¹²—R¹³—R¹⁴, which is substituted by 0, 1, 2, 3 or 4 substituents independently selected from R^{c}.

In another embodiment, in conjunction with any of the above or below embodiments, R² is selected from —R²¹, —R²¹—R²⁴, —R²¹—R²², —R²²—R²⁴, —R²¹—R²²—R²⁴, —R²¹—R²³—R²⁴, —R²²—R²³—R²⁴, —R²¹—R²³—R²²—R²⁴ and —R²¹—R²²—R²³—R²⁴, and of which is substituted by 0, 1, 2, 3 or 4 substituents independently selected from R^{c}.

Embodiment E: In another embodiment, in conjunction with any of the above or below embodiments, R² is selected from —R²¹, —R²¹—R²⁴, —R²¹—R²², —R²¹—R²²—R²⁴, —R²¹—R²³—R²⁴, —R²¹—R²³—R²²—R²⁴ and —R²¹—R²²—R²³—R²⁴, and of which is substituted by 0, 1, 2, 3 or 4 substituents independently selected from R^{c}.

Embodiment F: In another embodiment, in conjunction with any of the above or below embodiments, R² is R²¹, which is substituted by 0, 1, 2, 3 or 4 substituents independently selected from R^{c}.

In another embodiment, in conjunction with any of the above or below embodiments, R² is —R²¹—R²⁴, which is substituted by 0, 1, 2, 3 or 4 substituents independently selected from R^{c}.

In another embodiment, in conjunction with any of the above or below embodiments, R² is —R²¹—R²², which is substituted by 0, 1, 2, 3 or 4 substituents independently selected from R^{c}.

In another embodiment, in conjunction with any of the above or below embodiments, R² is —R²¹—R²²—R²⁴, which is substituted by 0, 1, 2, 3 or 4 substituents independently selected from R^{c}.

In another embodiment, in conjunction with any of the above or below embodiments, R² is —R²¹—R²³—R²⁴, which is substituted by 0, 1, 2, 3 or 4 substituents independently selected from R^{c}.

Embodiment G: In another embodiment, in conjunction with any of the above or below embodiments, R² is —R²¹—R²³—R²²—R²⁴, which is substituted by 0, 1, 2, 3 or 4 substituents independently selected from R^{c}.

In another embodiment, in conjunction with any of the above or below embodiments, R² is —R²¹—R²²—R²³—R²⁴, which is substituted by 0, 1, 2, 3 or 4 substituents independently selected from R^{c}.

In another embodiment, in conjunction with any of the above or below embodiments, R² is phenyl substituted by 1, 2, 3 or 4 substituents independently selected from R^{c}.

In another embodiment, in conjunction with any of the above or below embodiments, R² is a 2,5-disubstituted phenyl, wherein the two substituents are independently selected from R^{c}.

In another embodiment, in conjunction with any of the above or below embodiments, R² is a 2,5-disubstituted phenyl, wherein the two substituents are independently selected from C₁₋₂alkyl, halo and C₁₋₂haloalkyl.

In another embodiment, in conjunction with any of the above or below embodiments, R² is a 2,5-disubstituted phenyl, wherein the two substituents are independently selected from CH₃ and Cl.

In another embodiment, in conjunction with any of the above or below embodiments, R² is 2,5-dichlorophenyl.

In another embodiment, in conjunction with any of the above or below embodiments, R² is 2,5-dimethylphenyl.

Embodiment H: In another embodiment, in conjunction with any of the above or below embodiments, R^{3a} is selected from —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; and R^{3b}, R^{3c} and R^{3d} are each independently selected from H and R^{c}.

Embodiment I: In another embodiment, in conjunction with any of the above or below embodiments, R^{3b} is selected from —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; and R^{3a}, R^{3c} and R^{3d} are each independently selected from H and R^{c}.

Embodiment J: In another embodiment, in conjunction with any of the above or below embodiments, R^{3c} is selected from —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; and R^{3a}, R^{3b} and R^{3d} are each independently selected from H and R^{c}.

Embodiment K: In another embodiment, in conjunction with any of the above or below embodiments, R^{3d} is selected from —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; and R^{3a}, R^{3b} and R^{3c} are each independently selected from H and R^{c}.

Embodiment L: In another embodiment, in conjunction with any of the above or below embodiments, R^{3a}, R^{3b}, R^{3c} and R^{3d} are each independently selected from H and R^{c}.

In another embodiment, in conjunction with any of the above or below embodiments, R^{3a}, R^{3b}, R^{3c} and R^{3d} are each independently selected from R^{a}, R^{b} and R^{c};

In another embodiment, in conjunction with any of the above or below embodiments, R¹¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹¹ is independently at each instance an unsaturated 5- or 6-membered monocyclic or 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹¹ is independently at each instance an unsaturated 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹¹ is independently at each instance an unsaturated 5- or 6-membered monocyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹¹ is independently at each instance an unsaturated 5-membered monocyclic ring containing 1 atom selected from N, O and S.

In another embodiment, in conjunction with any of the above or below embodiments, R¹¹ is independently at each instance an unsaturated 6-membered monocyclic ring containing 0, 1 or 2 N atoms.

Embodiment M: In another embodiment, in conjunction with any of the above or below embodiments, R¹¹ is phenyl.

Embodiment N: In another embodiment, in conjunction with any of the above or below embodiments, R¹¹ is independently at each instance a saturated or unsaturated 5-, 6- or 7-membered monocyclic or 6-, 7-, 8-, 9-, 10- or 11-membered bicyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹¹ is independently at each instance an unsaturated 6-membered monocyclic ring containing 1 or 2 N atoms.

In another embodiment, in conjunction with any of the above or below embodiments, R¹¹ is a phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups.

In another embodiment, in conjunction with any of the above or below embodiments, R¹¹ is a phenyl, pyridyl, pyrimidinyl, thiophenyl, thiazolyl, oxazolyl, isoxazolyl, isothiazolyl, oxazolinyl, isoxazolinyl or thiazolinyl ring.

In another embodiment, in conjunction with any of the above or below embodiments, R¹¹ is pyridinyl, pyrimidinyl or pyridazinyl.

In another embodiment, in conjunction with any of the above or below embodiments, R¹² is independently at each instance C₁₋₈alkyl.

In another embodiment, in conjunction with any of the above or below embodiments, R¹² is independently at each instance C₁₋₄alkyl.

In another embodiment, in conjunction with any of the above or below embodiments, R¹² is independently at each instance C₂₋₄alkyl.

Embodiment O: In another embodiment, in conjunction with any of the above or below embodiments, R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R¹³ is independently at each instance —C(═O)—, —C(═O)O—, —C(═O)NR^{a}—, —C(═NR^{a})NR^{a}—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R³ is —O—.

In another embodiment, in conjunction with any of the above or below embodiments, R¹³ is —N(R^{a})—.

In another embodiment, in conjunction with any of the above or below embodiments, R¹³ is —N(R^{a})C(═O)—, —C(═O)NR^{a}—, —C(═O)O— or —OC(═O)—.

In another embodiment, in conjunction with any of the above or below embodiments, R¹³ is —O—, —N(R^{a})—, —N(R^{a})C(═O)—, —C(═O)NR^{a}—, —C(═O)O— or —OC(═O)—.

Embodiment P: In another embodiment, in conjunction with any of the above or below embodiments, R¹⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹⁴ is phenyl.

In another embodiment, in conjunction with any of the above or below embodiments, R¹⁴ is naphthyl.

In another embodiment, in conjunction with any of the above or below embodiments, R¹⁴ is independently at each instance a saturated or unsaturated 5-, 6- or 7-membered monocyclic or 6-, 7-, 8-, 9-, 10- or 11-membered bicyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹⁴ is independently at each instance a saturated or unsaturated 5-, 6- or 7-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹⁴ is independently at each instance a saturated 5- or 6-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R¹⁴ is independently at each instance a saturated 5- or 6-membered monocyclic ring containing 1 or 2 N atoms, wherein the carbon atoms of the ring are substituted by 0 or 1 oxo groups.

In another embodiment, in conjunction with any of the above or below embodiments, R¹⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups.

In another embodiment, in conjunction with any of the above or below embodiments, R¹⁴ is independently at each instance a phenyl, pyridyl, pyrimidinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups.

In another embodiment, in conjunction with any of the above or below embodiments, R¹⁴ is piperidinyl, piperazinyl or pyrrolidinyl.

In another embodiment, in conjunction with any of the above or below embodiments, R²¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²¹ is independently at each instance an unsaturated 5- or 6-membered monocyclic or 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²¹ is independently at each instance an unsaturated 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²¹ is independently at each instance an unsaturated 5- or 6-membered monocyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²¹ is independently at each instance an unsaturated 5-membered monocyclic ring containing 1 atom selected from N, O and S.

In another embodiment, in conjunction with any of the above or below embodiments, R²¹ is independently at each instance an unsaturated 6-membered monocyclic ring containing 0, 1 or 2 N atoms.

Embodiment Q: In another embodiment, in conjunction with any of the above or below embodiments, R²¹ is phenyl.

Embodiment R: In another embodiment, in conjunction with any of the above or below embodiments, R²¹ is independently at each instance a saturated or unsaturated 5-, 6- or 7-membered monocyclic or 6-, 7-, 8-, 9-, 10- or 11-membered bicyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²¹ is independently at each instance an unsaturated 6-membered monocyclic ring containing 1 or 2 N atoms.

In another embodiment, in conjunction with any of the above or below embodiments, R²¹ is phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups.

In another embodiment, in conjunction with any of the above or below embodiments, R²¹ is phenyl, pyridyl, pyrimidinyl, thiophenyl, thiazolyl, oxazolyl, isoxazolyl, isothiazolyl, oxazolinyl, isoxazolinyl or thiazolinyl ring.

In another embodiment, in conjunction with any of the above or below embodiments, R²¹ is pyridinyl, pyrimidinyl or pyridazinyl.

In another embodiment, in conjunction with any of the above or below embodiments, R¹² is independently at each instance C₁₋₈alkyl.

In another embodiment, in conjunction with any of the above or below embodiments, R²² is independently at each instance C₁₋₄alkyl.

In another embodiment, in conjunction with any of the above or below embodiments, R²² is independently at each instance C₂₋₄alkyl.

Embodiment S: In another embodiment, in conjunction with any of the above or below embodiments, R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R²³ is independently at each instance —C(═O)—, —C(═O)O—, —C(═O)NR^{a}—, —C(═NR^{a})NR^{a}—, —OC(═O)—, —OC(═O)NR^{a}—, —OC(═O)N(R^{a}S(═O)₂—, —OC₂₋₆alkylNR^{a}—, —OC₂₋₆alkylO—, —S—, —S(═O)—, —S(═O)₂—, —S(═O)₂NRₐ—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R²³ is —O—.

In another embodiment, in conjunction with any of the above or below embodiments, R²³ is —N(R^{a})—.

In another embodiment, in conjunction with any of the above or below embodiments, R²³ is —N(R^{a})C(═O)—, —C(═O)NR^{a}—, —C(═O)O— or —OC(═O)—.

In another embodiment, in conjunction with any of the above or below embodiments, R²³ is —O—, —N(R^{a})—, —N(R^{a})C(═O)—, —C(═O)NR^{a}—, —C(═O)O— or —OC(═O)—.

Embodiment T: In another embodiment, in conjunction with any of the above or below embodiments, R²⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²⁴ is phenyl.

In another embodiment, in conjunction with any of the above or below embodiments, R²⁴ is naphthyl.

In another embodiment, in conjunction with any of the above or below embodiments, R²⁴ is independently at each instance a saturated or unsaturated 5-, 6- or 7-membered monocyclic or 6-, 7-, 8-, 9-, 10- or 11-membered bicyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²⁴ is independently at each instance a saturated or unsaturated 5-, 6- or 7-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²⁴ is independently at each instance a saturated 5- or 6-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R²⁴ is independently at each instance a saturated 5- or 6-membered monocyclic ring containing 1 or 2 N atoms, wherein the carbon atoms of the ring are substituted by 0 or 1 oxo groups.

In another embodiment, in conjunction with any of the above or below embodiments, R²⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups.

In another embodiment, in conjunction with any of the above or below embodiments, R²⁴ is independently at each instance a phenyl, pyridyl, pyrimidinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups.

In another embodiment, in conjunction with any of the above or below embodiments, R²⁴ is piperidinyl, piperazinyl or pyrrolidinyl.

In another embodiment, in conjunction with any of the above or below embodiments, R³¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³¹ is independently at each instance an unsaturated 5- or 6-membered monocyclic or 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³¹ is independently at each instance an unsaturated 9- or 10-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³¹ is independently at each instance an unsaturated 5- or 6-membered monocyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³¹ is independently at each instance an unsaturated 5-membered monocyclic ring containing 1 atom selected from N, O and S.

In another embodiment, in conjunction with any of the above or below embodiments, R³¹ is independently at each instance an unsaturated 6-membered monocyclic ring containing 0, 1 or 2 N atoms.

Embodiment U: In another embodiment, in conjunction with any of the above or below embodiments, R³¹ is phenyl.

Embodiment V: In another embodiment, in conjunction with any of the above or below embodiments, R³¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³¹ is independently at each instance an unsaturated 6-membered monocyclic ring containing 1 or 2 N atoms.

In another embodiment, in conjunction with any of the above or below embodiments, R³¹ is independently at each instance a phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups.

In another embodiment, in conjunction with any of the above or below embodiments, R³¹ is pyridinyl, pyrimidinyl or pyridazinyl.

In another embodiment, in conjunction with any of the above or below embodiments, R³² is independently at each instance C₁₋₈alkyl.

In another embodiment, in conjunction with any of the above or below embodiments, R³² is independently at each instance C₁₋₄alkyl.

In another embodiment, in conjunction with any of the above or below embodiments, R³² is independently at each instance C₂₋₄alkyl.

Embodiment W: In another embodiment, in conjunction with any of the above or below embodiments, R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R³³ is independently at each instance —C(═O)—, —C(═O)O—, —C(═O)NR^{a}—, —C(═NR^{a})NR^{a}—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—.

In another embodiment, in conjunction with any of the above or below embodiments, R³³ is —O—.

In another embodiment, in conjunction with any of the above or below embodiments, R³³ is —N(R^{a})—.

In another embodiment, in conjunction with any of the above or below embodiments, R³³ is —N(R^{a})C(═O)—, —C(═O)NR^{a}—, —C(═O)O— or —OC(═O)—.

In another embodiment, in conjunction with any of the above or below embodiments, R³³ is —O—, —N(R^{a})—, —N(R^{a})C(═O)—, —C(═O)NR^{a}—, —C(═O)O— or —OC(═O)—.

Embodiment X: In another embodiment, in conjunction with any of the above or below embodiments, R³⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³⁴ is phenyl.

In another embodiment, in conjunction with any of the above or below embodiments, R³⁴ is naphthyl.

In another embodiment, in conjunction with any of the above or below embodiments, R³⁴ is independently at each instance a saturated or unsaturated 5-, 6- or 7-membered monocyclic or 6-, 7-, 8-, 9-, 10- or 11-membered bicyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³⁴ is independently at each instance a saturated or unsaturated 5-, 6- or 7-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³⁴ is independently at each instance a saturated 5- or 6-membered monocyclic ring containing 1, 2, 3 or 4 atoms selected from N, O and S, so long as the combination of O and S atoms is not greater than 2, wherein the carbon atoms of the ring are substituted by 0, 1 or 2 oxo groups.

In another embodiment, in conjunction with any of the above or below embodiments, R³⁴ is independently at each instance a saturated 5- or 6-membered monocyclic ring containing 1 or 2 N atoms, wherein the carbon atoms of the ring are substituted by 0 or 1 oxo groups.

In another embodiment, in conjunction with any of the above or below embodiments, R³⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;

In another embodiment, in conjunction with any of the above or below embodiments, R³⁴ is piperidinyl, piperazinyl or pyrrolidinyl.

As stated above, the above embodiments may be used inconjuction with other embodiments listed. The following table is a non-exclusive, non-limiting list of some of the combinations of embodiments. For the structure 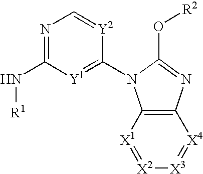
wherein

X¹ is C(R^{3a}), X² is C(R^{3b}), X³ is C(R^{3c}), X⁴ is C(R^{3d}), Y¹ is N, Y² is CH, R¹² and R²² are independently selected from C₁₋₄alkyl; R¹³, R²³ and R³³ are independently selected from Embodiments O, S and W, respectively; and R¹⁴, R²⁴ and R³⁴ are independently selected from Embodiments P, T and X, respectively.

| No. | R¹ | R¹¹ | R² | R²¹ | R^{3a-3d} | R³¹ |
|---|---|---|---|---|---|---|
| 1001 | A | M | E | Q | H | U |
| 1002 | A | M | E | Q | H | V |
| 1003 | A | M | E | Q | I | U |
| 1004 | A | M | E | Q | I | V |
| 1005 | A | M | E | Q | J | U |
| 1006 | A | M | E | Q | J | V |
| 1007 | A | M | E | Q | K | U |
| 1008 | A | M | E | Q | K | V |
| 1009 | A | M | E | Q | L | U |
| 1010 | A | M | E | Q | L | V |
| 1011 | A | M | E | R | H | U |
| 1012 | A | M | E | R | H | V |
| 1013 | A | M | E | R | I | U |
| 1014 | A | M | E | R | I | V |
| 1015 | A | M | E | R | J | U |
| 1016 | A | M | E | R | J | V |
| 1017 | A | M | E | R | K | U |
| 1018 | A | M | E | R | K | V |
| 1019 | A | M | E | R | L | U |
| 1020 | A | M | E | R | L | V |
| 1021 | A | M | F | Q | H | U |
| 1022 | A | M | F | Q | H | V |
| 1023 | A | M | F | Q | I | U |
| 1024 | A | M | F | Q | I | V |
| 1025 | A | M | F | Q | J | U |
| 1026 | A | M | F | Q | J | V |
| 1027 | A | M | F | Q | K | U |
| 1028 | A | M | F | Q | K | V |
| 1029 | A | M | F | Q | L | U |
| 1030 | A | M | F | Q | L | V |
| 1031 | A | M | F | R | H | U |
| 1032 | A | M | F | R | H | V |
| 1033 | A | M | F | R | I | U |
| 1034 | A | M | F | R | I | V |
| 1035 | A | M | F | R | J | U |
| 1036 | A | M | F | R | J | V |
| 1037 | A | M | F | R | K | U |
| 1038 | A | M | F | R | K | V |
| 1039 | A | M | F | R | L | U |
| 1040 | A | M | F | R | L | V |
| 1041 | A | M | G | Q | H | U |
| 1042 | A | M | G | Q | H | V |
| 1043 | A | M | G | Q | I | U |
| 1044 | A | M | G | Q | I | V |
| 1045 | A | M | G | Q | J | U |
| 1046 | A | M | G | Q | J | V |
| 1047 | A | M | G | Q | K | U |
| 1048 | A | M | G | Q | K | V |
| 1049 | A | M | G | Q | L | U |
| 1050 | A | M | G | Q | L | V |
| 1051 | A | M | G | R | H | U |
| 1052 | A | M | G | R | H | V |
| 1053 | A | M | G | R | I | U |
| 1054 | A | M | G | R | I | V |
| 1055 | A | M | G | R | J | U |
| 1056 | A | M | G | R | J | V |
| 1057 | A | M | G | R | K | U |
| 1058 | A | M | G | R | K | V |
| 1059 | A | M | G | R | L | U |
| 1060 | A | M | G | R | L | V |
| 1061 | A | N | E | Q | H | U |
| 1062 | A | N | E | Q | H | V |
| 1063 | A | N | E | Q | I | U |
| 1064 | A | N | E | Q | I | V |
| 1065 | A | N | E | Q | J | U |
| 1066 | A | N | E | Q | J | V |
| 1067 | A | N | E | Q | K | U |
| 1068 | A | N | E | Q | K | V |
| 1069 | A | N | E | Q | L | U |
| 1070 | A | N | E | Q | L | V |
| 1071 | A | N | E | R | H | U |
| 1072 | A | N | E | R | H | V |
| 1073 | A | N | E | R | I | U |
| 1074 | A | N | E | R | I | V |
| 1075 | A | N | E | R | J | U |
| 1076 | A | N | E | R | J | V |
| 1077 | A | N | E | R | K | U |
| 1078 | A | N | E | R | K | V |
| 1079 | A | N | E | R | L | U |
| 1080 | A | N | E | R | L | V |
| 1081 | A | N | F | Q | H | U |
| 1082 | A | N | F | Q | H | V |
| 1083 | A | N | F | Q | I | U |
| 1084 | A | N | F | Q | I | V |
| 1085 | A | N | F | Q | J | U |
| 1086 | A | N | F | Q | J | V |
| 1087 | A | N | F | Q | K | U |
| 1088 | A | N | F | Q | K | V |
| 1089 | A | N | F | Q | L | U |
| 1090 | A | N | F | Q | L | V |
| 1091 | A | N | F | R | H | U |
| 1092 | A | N | F | R | H | V |
| 1093 | A | N | F | R | I | U |
| 1094 | A | N | F | R | I | V |
| 1095 | A | N | F | R | J | U |
| 1096 | A | N | F | R | J | V |
| 1097 | A | N | F | R | K | U |
| 1098 | A | N | F | R | K | V |
| 1099 | A | N | F | R | L | U |
| 1100 | A | N | F | R | L | V |
| 1101 | A | N | G | Q | H | U |
| 1102 | A | N | G | Q | H | V |
| 1103 | A | N | G | Q | I | U |
| 1104 | A | N | G | Q | I | V |
| 1105 | A | N | G | Q | J | U |
| 1106 | A | N | G | Q | J | V |
| 1107 | A | N | G | Q | K | U |
| 1108 | A | N | G | Q | K | V |
| 1109 | A | N | G | Q | L | U |
| 1110 | A | N | G | Q | L | V |
| 1111 | A | N | G | R | H | U |
| 1112 | A | N | G | R | H | V |
| 1113 | A | N | G | R | I | U |
| 1114 | A | N | G | R | I | V |
| 1115 | A | N | G | R | J | U |
| 1116 | A | N | G | R | J | V |
| 1117 | A | N | G | R | K | U |
| 1118 | A | N | G | R | K | V |
| 1119 | A | N | G | R | L | U |
| 1120 | A | N | G | R | L | V |
| 1121 | B | M | E | Q | H | U |
| 1122 | B | M | E | Q | H | V |
| 1123 | B | M | E | Q | I | U |
| 1124 | B | M | E | Q | I | V |
| 1125 | B | M | E | Q | J | U |
| 1126 | B | M | E | Q | J | V |
| 1127 | B | M | E | Q | K | U |
| 1128 | B | M | E | Q | K | V |
| 1129 | B | M | E | Q | L | U |
| 1130 | B | M | E | Q | L | V |
| 1131 | B | M | E | R | H | U |
| 1132 | B | M | E | R | H | V |
| 1133 | B | M | E | R | I | U |
| 1134 | B | M | E | R | I | V |
| 1135 | B | M | E | R | J | U |
| 1136 | B | M | E | R | J | V |
| 1137 | B | M | E | R | K | U |
| 1138 | B | M | E | R | K | V |
| 1139 | B | M | E | R | L | U |
| 1140 | B | M | E | R | L | V |
| 1141 | B | M | F | Q | H | U |
| 1142 | B | M | F | Q | H | V |
| 1143 | B | M | F | Q | I | U |
| 1144 | B | M | F | Q | I | V |
| 1145 | B | M | F | Q | J | U |
| 1146 | B | M | F | Q | J | V |
| 1147 | B | M | F | Q | K | U |
| 1148 | B | M | F | Q | K | V |
| 1149 | B | M | F | Q | L | U |
| 1150 | B | M | F | Q | L | V |
| 1151 | B | M | F | R | H | U |
| 1152 | B | M | F | R | H | V |
| 1153 | B | M | F | R | I | U |
| 1154 | B | M | F | R | I | V |
| 1155 | B | M | F | R | J | U |
| 1156 | B | M | F | R | J | V |
| 1157 | B | M | F | R | K | U |
| 1158 | B | M | F | R | K | V |
| 1159 | B | M | F | R | L | U |
| 1160 | B | M | F | R | L | V |
| 1161 | B | M | G | Q | H | U |
| 1162 | B | M | G | Q | H | V |
| 1163 | B | M | G | Q | I | U |
| 1164 | B | M | G | Q | I | V |
| 1165 | B | M | G | Q | J | U |
| 1166 | B | M | G | Q | J | V |
| 1167 | B | M | G | Q | K | U |
| 1168 | B | M | G | Q | K | V |
| 1169 | B | M | G | Q | L | U |
| 1170 | B | M | G | Q | L | V |
| 1171 | B | M | G | R | H | U |
| 1172 | B | M | G | R | H | V |
| 1173 | B | M | G | R | I | U |
| 1174 | B | M | G | R | I | V |
| 1175 | B | M | G | R | J | U |
| 1176 | B | M | G | R | J | V |
| 1177 | B | M | G | R | K | U |
| 1178 | B | M | G | R | K | V |
| 1179 | B | M | G | R | L | U |
| 1180 | B | M | G | R | L | V |
| 1181 | B | N | E | Q | H | U |
| 1182 | B | N | E | Q | H | V |
| 1183 | B | N | E | Q | I | U |
| 1184 | B | N | E | Q | I | V |
| 1185 | B | N | E | Q | J | U |
| 1186 | B | N | E | Q | J | V |
| 1187 | B | N | E | Q | K | U |
| 1188 | B | N | E | Q | K | V |
| 1189 | B | N | E | Q | L | U |
| 1190 | B | N | E | Q | L | V |
| 1191 | B | N | E | R | H | U |
| 1192 | B | N | E | R | H | V |
| 1193 | B | N | E | R | I | U |
| 1194 | B | N | E | R | I | V |
| 1195 | B | N | E | R | J | U |
| 1196 | B | N | E | R | J | V |
| 1197 | B | N | E | R | K | U |
| 1198 | B | N | E | R | K | V |
| 1199 | B | N | E | R | L | U |
| 1200 | B | N | E | R | L | V |
| 1201 | B | N | F | Q | H | U |
| 1202 | B | N | F | Q | H | V |
| 1203 | B | N | F | Q | I | U |
| 1204 | B | N | F | Q | I | V |
| 1205 | B | N | F | Q | J | U |
| 1206 | B | N | F | Q | J | V |
| 1207 | B | N | F | Q | K | U |
| 1208 | B | N | F | Q | K | V |
| 1209 | B | N | F | Q | L | U |
| 1210 | B | N | F | Q | L | V |
| 1211 | B | N | F | R | H | U |
| 1212 | B | N | F | R | H | V |
| 1213 | B | N | F | R | I | U |
| 1214 | B | N | F | R | I | V |
| 1215 | B | N | F | R | J | U |
| 1216 | B | N | F | R | J | V |
| 1217 | B | N | F | R | K | U |
| 1218 | B | N | F | R | K | V |
| 1219 | B | N | F | R | L | U |
| 1220 | B | N | F | R | L | V |
| 1221 | B | N | G | Q | H | U |
| 1222 | B | N | G | Q | H | V |
| 1223 | B | N | G | Q | I | U |
| 1224 | B | N | G | Q | I | V |
| 1225 | B | N | G | Q | J | U |
| 1226 | B | N | G | Q | J | V |
| 1227 | B | N | G | Q | K | U |
| 1228 | B | N | G | Q | K | V |
| 1229 | B | N | G | Q | L | U |
| 1230 | B | N | G | Q | L | V |
| 1231 | B | N | G | R | H | U |
| 1232 | B | N | G | R | H | V |
| 1233 | B | N | G | R | I | U |
| 1234 | B | N | G | R | I | V |
| 1235 | B | N | G | R | J | U |
| 1236 | B | N | G | R | J | V |
| 1237 | B | N | G | R | K | U |
| 1238 | B | N | G | R | K | V |
| 1239 | B | N | G | R | L | U |
| 1240 | B | N | G | R | L | V |
| 1241 | C | M | E | Q | H | U |
| 1242 | C | M | E | Q | H | V |
| 1243 | C | M | E | Q | I | U |
| 1244 | C | M | E | Q | I | V |
| 1245 | C | M | E | Q | J | U |
| 1246 | C | M | E | Q | J | V |
| 1247 | C | M | E | Q | K | U |
| 1248 | C | M | E | Q | K | V |
| 1249 | C | M | E | Q | L | U |
| 1250 | C | M | E | Q | L | V |
| 1251 | C | M | E | R | H | U |
| 1252 | C | M | E | R | H | V |
| 1253 | C | M | E | R | I | U |
| 1254 | C | M | E | R | I | V |
| 1255 | C | M | E | R | J | U |
| 1256 | C | M | E | R | J | V |
| 1257 | C | M | E | R | K | U |
| 1258 | C | M | E | R | K | V |
| 1259 | C | M | E | R | L | U |
| 1260 | C | M | E | R | L | V |
| 1261 | C | M | F | Q | H | U |
| 1262 | C | M | F | Q | H | V |
| 1263 | C | M | F | Q | I | U |
| 1264 | C | M | F | Q | I | V |
| 1265 | C | M | F | Q | J | U |
| 1266 | C | M | F | Q | J | V |
| 1267 | C | M | F | Q | K | U |
| 1268 | C | M | F | Q | K | V |
| 1269 | C | M | F | Q | L | U |
| 1270 | C | M | F | Q | L | V |
| 1271 | C | M | F | R | H | U |
| 1272 | C | M | F | R | H | V |
| 1273 | C | M | F | R | I | U |
| 1274 | C | M | F | R | I | V |
| 1275 | C | M | F | R | J | U |
| 1276 | C | M | F | R | J | V |
| 1277 | C | M | F | R | K | U |
| 1278 | C | M | F | R | K | V |
| 1279 | C | M | F | R | L | U |
| 1280 | C | M | F | R | L | V |
| 1281 | C | M | G | Q | H | U |
| 1282 | C | M | G | Q | H | V |
| 1283 | C | M | G | Q | I | U |
| 1284 | C | M | G | Q | I | V |
| 1285 | C | M | G | Q | J | U |
| 1286 | C | M | G | Q | J | V |
| 1287 | C | M | G | Q | K | U |
| 1288 | C | M | G | Q | K | V |
| 1289 | C | M | G | Q | L | U |
| 1290 | C | M | G | Q | L | V |
| 1291 | C | M | G | R | H | U |
| 1292 | C | M | G | R | H | V |
| 1293 | C | M | G | R | I | U |
| 1294 | C | M | G | R | I | V |
| 1295 | C | M | G | R | J | U |
| 1296 | C | M | G | R | J | V |
| 1297 | C | M | G | R | K | U |
| 1298 | C | M | G | R | K | V |
| 1299 | C | M | G | R | L | U |
| 1300 | C | M | G | R | L | V |
| 1301 | C | N | E | Q | H | U |
| 1302 | C | N | E | Q | H | V |
| 1303 | C | N | E | Q | I | U |
| 1304 | C | N | E | Q | I | V |
| 1305 | C | N | E | Q | J | U |
| 1306 | C | N | E | Q | J | V |
| 1307 | C | N | E | Q | K | U |
| 1308 | C | N | E | Q | K | V |
| 1309 | C | N | E | Q | L | U |
| 1310 | C | N | E | Q | L | V |
| 1311 | C | N | E | R | H | U |
| 1312 | C | N | E | R | H | V |
| 1313 | C | N | E | R | I | U |
| 1314 | C | N | E | R | I | V |
| 1315 | C | N | E | R | J | U |
| 1316 | C | N | E | R | J | V |
| 1317 | C | N | E | R | K | U |
| 1318 | C | N | E | R | K | V |
| 1319 | C | N | E | R | L | U |
| 1320 | C | N | E | R | L | V |
| 1321 | C | N | F | Q | H | U |
| 1322 | C | N | F | Q | H | V |
| 1323 | C | N | F | Q | I | U |
| 1324 | C | N | F | Q | I | V |
| 1325 | C | N | F | Q | J | U |
| 1326 | C | N | F | Q | J | V |
| 1327 | C | N | F | Q | K | U |
| 1328 | C | N | F | Q | K | V |
| 1329 | C | N | F | Q | L | U |
| 1330 | C | N | F | Q | L | V |
| 1331 | C | N | F | R | H | U |
| 1332 | C | N | F | R | H | V |
| 1333 | C | N | F | R | I | U |
| 1334 | C | N | F | R | I | V |
| 1335 | C | N | F | R | J | U |
| 1336 | C | N | F | R | J | V |
| 1337 | C | N | F | R | K | U |
| 1338 | C | N | F | R | K | V |
| 1339 | C | N | F | R | L | U |
| 1340 | C | N | F | R | L | V |
| 1341 | C | N | G | Q | H | U |
| 1342 | C | N | G | Q | H | V |
| 1343 | C | N | G | Q | I | U |
| 1344 | C | N | G | Q | I | V |
| 1345 | C | N | G | Q | J | U |
| 1346 | C | N | G | Q | J | V |
| 1347 | C | N | G | Q | K | U |
| 1348 | C | N | G | Q | K | V |
| 1349 | C | N | G | Q | L | U |
| 1350 | C | N | G | Q | L | V |
| 1351 | C | N | G | R | H | U |
| 1352 | C | N | G | R | H | V |
| 1353 | C | N | G | R | I | U |
| 1354 | C | N | G | R | I | V |
| 1355 | C | N | G | R | J | U |
| 1356 | C | N | G | R | J | V |
| 1357 | C | N | G | R | K | U |
| 1358 | C | N | G | R | K | V |
| 1359 | C | N | G | R | L | U |
| 1360 | C | N | G | R | L | V |
| 1361 | D | M | E | Q | H | U |
| 1362 | D | M | E | Q | H | V |
| 1363 | D | M | E | Q | I | U |
| 1364 | D | M | E | Q | I | V |
| 1365 | D | M | E | Q | J | U |
| 1366 | D | M | E | Q | J | V |
| 1367 | D | M | E | Q | K | U |
| 1368 | D | M | E | Q | K | V |
| 1369 | D | M | E | Q | L | U |
| 1370 | D | M | E | Q | L | V |
| 1371 | D | M | E | R | H | U |
| 1372 | D | M | E | R | H | V |
| 1373 | D | M | E | R | I | U |
| 1374 | D | M | E | R | I | V |
| 1375 | D | M | E | R | J | U |
| 1376 | D | M | E | R | J | V |
| 1377 | D | M | E | R | K | U |
| 1378 | D | M | E | R | K | V |
| 1379 | D | M | E | R | L | U |
| 1380 | D | M | E | R | L | V |
| 1381 | D | M | F | Q | H | U |
| 1382 | D | M | F | Q | H | V |
| 1383 | D | M | F | Q | I | U |
| 1384 | D | M | F | Q | I | V |
| 1385 | D | M | F | Q | J | U |
| 1386 | D | M | F | Q | J | V |
| 1387 | D | M | F | Q | K | U |
| 1388 | D | M | F | Q | K | V |
| 1389 | D | M | F | Q | L | U |
| 1390 | D | M | F | Q | L | V |
| 1391 | D | M | F | R | H | U |
| 1392 | D | M | F | R | H | V |
| 1393 | D | M | F | R | I | U |
| 1394 | D | M | F | R | I | V |
| 1395 | D | M | F | R | J | U |
| 1396 | D | M | F | R | J | V |
| 1397 | D | M | F | R | K | U |
| 1398 | D | M | F | R | K | V |
| 1399 | D | M | F | R | L | U |
| 1400 | D | M | F | R | L | V |
| 1401 | D | M | G | Q | H | U |
| 1402 | D | M | G | Q | H | V |
| 1403 | D | M | G | Q | I | U |
| 1404 | D | M | G | Q | I | V |
| 1405 | D | M | G | Q | J | U |
| 1406 | D | M | G | Q | J | V |
| 1407 | D | M | G | Q | K | U |
| 1408 | D | M | G | Q | K | V |
| 1409 | D | M | G | Q | L | U |
| 1410 | D | M | G | Q | L | V |
| 1411 | D | M | G | R | H | U |
| 1412 | D | M | G | R | H | V |
| 1413 | D | M | G | R | I | U |
| 1414 | D | M | G | R | I | V |
| 1415 | D | M | G | R | J | U |
| 1416 | D | M | G | R | J | V |
| 1417 | D | M | G | R | K | U |
| 1418 | D | M | G | R | K | V |
| 1419 | D | M | G | R | L | U |
| 1420 | D | M | G | R | L | V |
| 1421 | D | N | E | Q | H | U |
| 1422 | D | N | E | Q | H | V |
| 1423 | D | N | E | Q | I | U |
| 1424 | D | N | E | Q | I | V |
| 1425 | D | N | E | Q | J | U |
| 1426 | D | N | E | Q | J | V |
| 1427 | D | N | E | Q | K | U |
| 1428 | D | N | E | Q | K | V |
| 1429 | D | N | E | Q | L | U |
| 1430 | D | N | E | Q | L | V |
| 1431 | D | N | E | R | H | U |
| 1432 | D | N | E | R | H | V |
| 1433 | D | N | E | R | I | U |
| 1434 | D | N | E | R | I | V |
| 1435 | D | N | E | R | J | U |
| 1436 | D | N | E | R | J | V |
| 1437 | D | N | E | R | K | U |
| 1438 | D | N | E | R | K | V |
| 1439 | D | N | E | R | L | U |
| 1440 | D | N | E | R | L | V |
| 1441 | D | N | F | Q | H | U |
| 1442 | D | N | F | Q | H | V |
| 1443 | D | N | F | Q | I | U |
| 1444 | D | N | F | Q | I | V |
| 1445 | D | N | F | Q | J | U |
| 1446 | D | N | F | Q | J | V |
| 1447 | D | N | F | Q | K | U |
| 1448 | D | N | F | Q | K | V |
| 1449 | D | N | F | Q | L | U |
| 1450 | D | N | F | Q | L | V |
| 1451 | D | N | F | R | H | U |
| 1452 | D | N | F | R | H | V |
| 1453 | D | N | F | R | I | U |
| 1454 | D | N | F | R | I | V |
| 1455 | D | N | F | R | J | U |
| 1456 | D | N | F | R | J | V |
| 1457 | D | N | F | R | K | U |
| 1458 | D | N | F | R | K | V |
| 1459 | D | N | F | R | L | U |
| 1460 | D | N | F | R | L | V |
| 1461 | D | N | G | Q | H | U |
| 1462 | D | N | G | Q | H | V |
| 1463 | D | N | G | Q | I | U |
| 1464 | D | N | G | Q | I | V |
| 1465 | D | N | G | Q | J | U |
| 1466 | D | N | G | Q | J | V |
| 1467 | D | N | G | Q | K | U |
| 1468 | D | N | G | Q | K | V |
| 1469 | D | N | G | Q | L | U |
| 1470 | D | N | G | Q | L | V |
| 1471 | D | N | G | R | H | U |
| 1472 | D | N | G | R | H | V |
| 1473 | D | N | G | R | I | U |
| 1474 | D | N | G | R | I | V |
| 1475 | D | N | G | R | J | U |
| 1476 | D | N | G | R | J | V |
| 1477 | D | N | G | R | K | U |
| 1478 | D | N | G | R | K | V |
| 1479 | D | N | G | R | L | U |
| 1480 | D | N | G | R | L | V |

Another aspect of the invention relates to a pharmaceutical composition comprising a compound according to any one of the above embodiments and a pharmaceutically acceptable carrier.

Another aspect of the invention relates to a method of treatment of inflammation comprising administering a therapeutically-effective amount of a compound according to any one of the above embodiments.

Another aspect of the invention relates to a method of inhibition of T cell activation and proliferation in a mammal in need thereof, comprising administering a therapeutically-effective amount of a compound according to any one of the above embodiments.

Another aspect of the invention relates to a method of treatment of arthritis, rheumatoid arthritis, psoriatic arthritis, or osteoarthritis in a mammal comprising administering a therapeutically-effective amount of a compound according to any one of the above embodiments.

Another aspect of the invention relates to a method of treatment of organ transplant, acute transplant or heterograft or homograft rejection, or transplantation tolerance induction in a mammal comprising administering a therapeutically-effective amount of a compound according to any one of the above embodiments.

Another aspect of the invention relates to a method of treatment of ischemic or reperfusion injury, myocardial infarction, or stroke in a mammal in need thereof, comprising administering a therapeutically-effective amount of a compound according to any one of the above embodiments.

Another aspect of the invention relates to a method of treatment of multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea or atopic dermatitis in a mammal comprising administering a therapeutically-effective amount of a compound according to any one of the above embodiments.

Another aspect of the invention relates to a method of treatment of colon carcinoma or thymoma in a mammal comprising administering a therapeutically-effective amount of a compound according to any one of the above embodiments.

Another aspect of the invention relates to the manufacture of a medicament comprising a compound according to any one of the above embodiments.

Another aspect of the invention relates to the manufacture of a medicament for the treatment of inflammation comprising a therapeutically-effective amount of a compound according to any one of the above embodiments.

Another aspect of the invention relates to the manufacture of a medicament for the inhibition of T cell activation and proliferation in a mammal in need thereof, comprising a therapeutically-effective amount of a compound according to any one of the above embodiments.

Another aspect of the invention relates to the manufacture of a medicament for the treatment of arthritis, rheumatoid arthritis, psoriatic arthritis, or osteoarthritis in a mammal comprising a therapeutically-effective amount of a compound according to any one of the above embodiments.

Another aspect of the invention relates to the manufacture of a medicament for the treatment of organ transplant, acute transplant or heterograft or homograft rejection, or transplantation tolerance induction in a mammal comprising a therapeutically-effective amount of a compound according to any one of the above embodiments.

Another aspect of the invention relates to the manufacture of a medicament for the treatment of ischemic or reperfusion injury, myocardial infarction, or stroke in a mammal in need thereof, comprising a therapeutically-effective amount of a compound according to any one of the above embodiments.

Another aspect of the invention relates to the manufacture of a medicament for the treatment of multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea or atopic dermatitis in a mammal comprising a therapeutically-effective amount of a compound according to any one of the above embodiments.

Another aspect of the invention relates to the manufacture of a medicament for the treatment of colon carcinoma or thymoma in a mammal comprising a therapeutically-effective amount of a compound according to any one of the above embodiments.

Another aspect of the invention relates to a method of making a compound as described herein, comprising the steps of:
reacting a compound having the structure 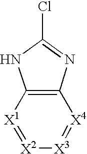
with dialkylcarbonate to give 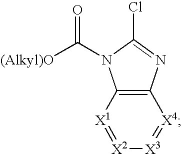
reacting the product with R²OH to give 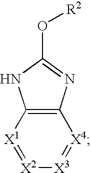
reacting the formed product with 2,4-dihalopyrimidine to give 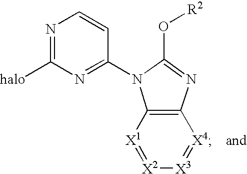
reacting the halopyrimidine with HN—R¹ in the presence of acid to give

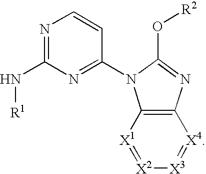

The compounds of this invention may have in general several asymmetric centers and are typically depicted in the form of racemic mixtures. This invention is intended to encompass racemic mixtures, partially racemic mixtures and separate enantiomers and diasteromers.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups from the genus.

Unless otherwise specified, the following definitions apply to terms found in the specification and claims:

“Aryl” means a phenyl or naphthyl radical, wherein the phenyl may be fused with a C₃₋₄cycloalkyl bridge.

“Benzo group”, alone or in combination, means the divalent radical C₄H₄═, one representation of which is —CH═CH—CH═CH—, that when vicinally attached to another ring forms a benzene-like ring—for example tetrahydronaphthylene, indole and the like.

“C_{α-β}alkyl” means an alkyl group comprising from α to β carbon atoms in a branched, cyclical or linear relationship or any combination of the three. The alkyl groups described in this section may also contain double or triple bonds. Examples of C₁₋₈alkyl include, but are not limited to the following:

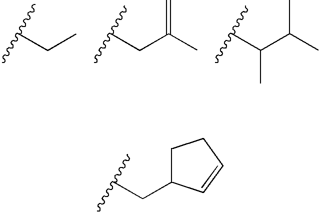

“Halogen” and “halo” mean a halogen atoms selected from F, Cl, Br and I.

“C_{α-β}haloalkyl” means an alkyl group, as described above, wherein any number—at least one—of the hydrogen atoms attached to the alkyl chain are replaced by F, Cl, Br or I.

“Heterocycle” means a ring comprising at least one carbon atom and at least one other atom selected from N, O and S. Examples of heterocycles that may be found in the claims include, but are not limited to, the following:

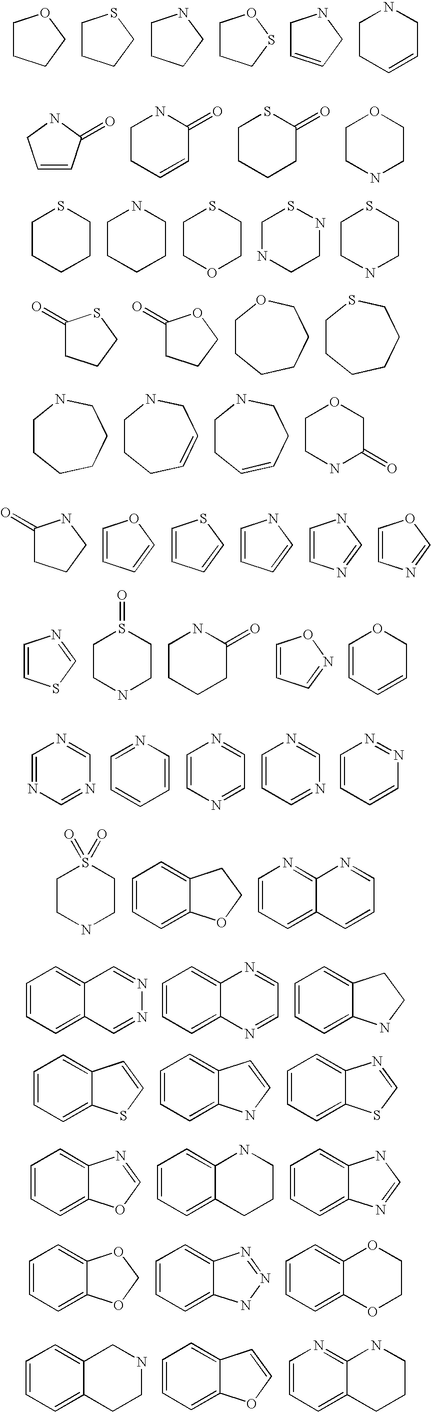

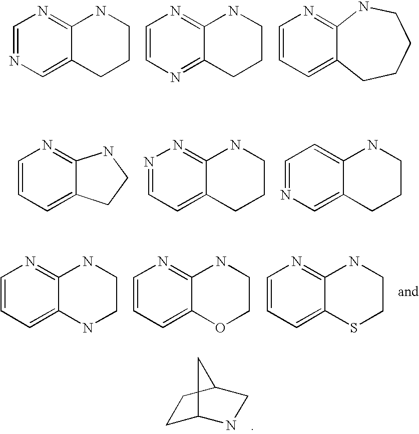

“Saturated or unsaturated” means a substitutent that is completely saturated, completely unsaturated, or has any degree of unsaturation in between. Examples of a saturated or unsaturated 6-membered ring carbocycle would include phenyl, cyclohexyl, cyclohexenyl and cyclohexadienyl.

Substituents, including rings and alkyl groups, may be either monovalent or polyvalent depending on the context of their usage. For example, if description contained the group R^{α}—R^{β—R}^{γ} and R^{β} was defined as C₁₋₆alkyl, then the R^{β} alkyl would be considered polyvalent because it must be bonded to at least R^{α} and R^{γ}. Alternatively, if R^{γ} was defined as C₁₋₆alkyl, then the R^{γ} alkyl would be monovalent (excepting any further substitution language).

“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulphonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.

“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, tri-fluoroacetyl, tri-chloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, tri-isopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-tri-silyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′═O, S, NR), and the like, which are illustrated in the following examples: 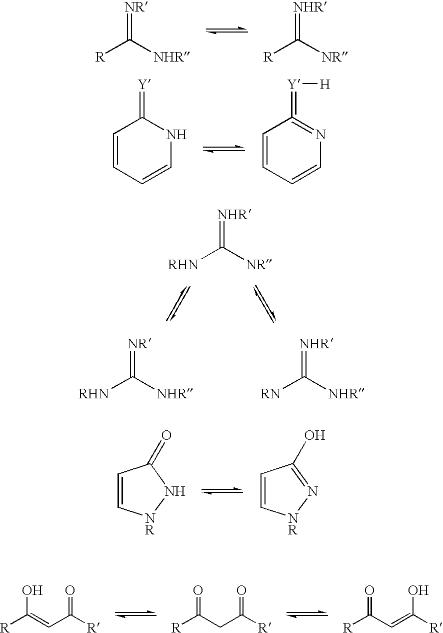
and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl).

Amines have been masked as arylcarbonyloxymethyl substituted derivatives which are cleaved by esterases in vivo releasing the free drug and formaldehyde (Bundgaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

“Cytokine” means a secreted protein that affects the functions of other cells, particularly as it relates to the modulation of interactions between cells of the immune system or cells involved in the inflammatory response. Examples of cytokines include but are not limited to interleukin 1 (IL-1), preferably IL-1β, interleukin 6 (IL-6), interleukin 8 (IL-8) and TNF, preferably TNF-α (tumor necrosis factor-α).

Synthesis

Compounds according to the invention can be synthesized according to one or more of the following methods. It should be noted that the general procedures are shown as it relates to preparation of compounds having unspecified stereochemistry. However, such procedures are generally applicable to those compounds of a specific stereochemistry, e.g., where the stereochemistry about a group is (S) or (R). In addition, the compounds having one stereochemistry (e.g., (R)) can often be utilized to produce those having opposite stereochemistry (i.e., (S)) using well-known methods, for example, by inversion.

The following Examples are presented for illustrative purposes only and are not intended, nor should they be construed, as limiting the invention in any manner. Those skilled in the art will appreciate that modifications and variations of the compounds disclosed herein can be made without violating the spirit or scope of the present invention.
Aniline Synthesis 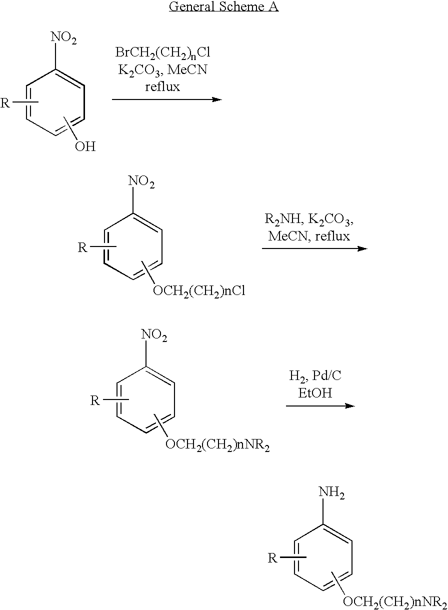
General Method A

### 3-(4-Nitrophenoxy)propyl chloride

Nitrophenol (10 g, 72 mmol) was dissolved in acetonitrile (100 mL) and potassium carbonate (24.9 g, 180 mmol) added followed by bromochloropropane (113.2 g, 720 mmol). The mixture was heated and stirred under reflux overnight. The reaction was cooled to room temperature, the solid was then filtered off and the solvent evaporated under reduced pressure, taking care to remove all excess alkylating agent, to give the title compound.

### N,N-dimethyl-3-(4-nitrophenoxy)propylamine

A mixture of 3-(4-nitrophenoxy)propyl chloride (2 g, 9.27 mmol), potassium carbonate (7.69 g, 46.4 mmol) and acetonitrile (15 mL) were stirred in a sealed tube and dimethylamine hydrochloride (3.78 g, 46.4 mmol) added quickly. The mixture was stirred and heated overnight at 80° C. The mixture was cooled well before opening the pressure tube, then water and dichloromethane were added and the aqueous layer was extracted with dichloromethane. The combined organics were dried and evaporated giving the title product. ¹H NMR (400 MHz, CDCl₃): 1.95 (2H, t, J 7 Hz); 2.2 (6H, s); 2.35-2.45 (2H, m); 4.05 (2H, t, J 7 Hz); 6.9 (2H, d, J 8 Hz); 8.1 (2H, d, J 8 Hz)

### N,N-dimethyl-3-(4-aminophenoxy)propylamine

N,N-dimethyl-3-(4-nitrophenoxy)propylamine (4.4 g, 19.6 mmol) was hydrogenated over Pd (10% on C, 0.4 g) in ethanol (ca 50 mL) for 16 h. The catalysts was filtered off and the solvent removed under reduced pressure to afford the title compound as a brown oil. ¹H NMR (400 MHz, dmso-d6): 1.95 (2H, t, J 6.5 Hz); 2.25 (6H, s); 2.35-2.45 (2H, m); 3.95 (2H, t, J 6.5 Hz); 4.7 (2H, bs); 6.9 (2H, d, J 8 Hz); 8.1 (2H, d, J 8 Hz); 6.65 (2H, d, J 8 Hz); 6.75 (2H, d, J 8 Hz)
General Scheme B 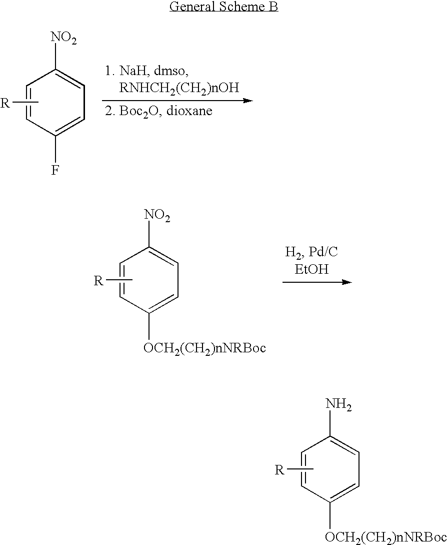
General Method B

### Isopropyl-[2-(4-nitrophenoxy)ethyl]amine

Deprotonation of DMSO (anhydrous, 5 mL) was effected with NaH (0.40 g, 60 wt % in mineral oil, 10 mmol) over 30 min at 40° C. with stirring under a nitrogen atmosphere. When 2-isopropylaminoethanol (1.15 mL, 10 mmol) was added to the solution of the DMSO anion at room temperature, some effervescence occurred. 4-Fluoronitrobenzene (1.06 mL, 10 mmol) was added after 10 min and the dark red solution was then stirred at room temperature for further 20 min. The reaction was diluted with dichloromethane (100 mL), washed with water (50 mL) and then extracted twice with 3M HCl (100 mL). The combined acidic extracts were washed once with dichloromethane (50 mL). Ethyl acetate (125 mL) was then added and the mixture was cooled to 6-8° C. before the aqueous layer was adjusted to pH 11 by gradual addition of 5M aq. NaOH (ca. 150 mL), with vigorous stirring. The organic layer was separated and washed twice with water (50 mL) dried over magnesium sulfate, and concentrated in vacuo at 35° C. to afford the title compound as a yellow oil. ¹H NMR (400 MHz, CDCl₃): 1.10 (6H, d, J 6.25), 2.88 (1H, m, J 6.25), 3.04 (2H, t, J 5.2), 4.16 (2H, t, J 5.2), 6.96 (2H, d, J 9.3), 8.18 (2H, d, J 9.3); MS: 225

### Isopropyl-[2-(4-nitrophenoxy)ethyl]carbamic acid tert-butyl ester

Isopropyl-[2-(4-nitrophenoxy)ethyl]amine (1.80 g, 8.05 mmol) was dissolved in 1,4-dioxane (containing 1% water, 20 mL) and cooled to 0-5° C. Di-tert-butyldicarbonate (1.76 g, 8.05 mmol) was added slowly with vigorous stirring. The reaction was stirred at 0° C. for 0.5 h, then at room temperature for 20 h. The solvent was removed in vacuo and the residue taken up into EtOAc. The organic layer was washed twice with water (25 mL), the aqueous washes are extracted back with EtOAc (25 mL). The combined organic extracts were washed twice with 0.3 M HCl (25 mL), then brine and are dried over sodium sulfate. The solvent was removed in vacuo to afford a yellow solid, which was recrystallised from hot n-hexane to give the crystalline title compound as fine, light-yellow needles. ¹H NMR: (400 MHz, CDCl₃): 1.06 (6H, d, J 6.8), 1.37 (9H, s), 3.90 (2H, bm, 2H), 4.06 (2H, bm), 4.26 (1H, bm), 6.86 (2H, d, J 9.0), 8.09 (2H, d, J 9.2). MS: 225 [M+H⁺-Boc]).

### Isopropyl-[2-(4-aminophenoxy)ethyl]carbamic acid tert-butyl ester

A solution of isopropyl-[2-(4-aminophenoxy)ethyl]carbamic acid tert-butyl ester (2.09 g, 6.45 mmol) in ethanol/tetrahydrofuran (30 mL, 2:1) was reduced over palladium on carbon (10 wt %, 50% wet, 0.4 g) with hydrogen under atmospheric pressure at room temperature for 20 h. The catalyst was separated by filtration through celite. The solvent was removed in vacuo to afford the title compound as a red oil. ¹H NMR: (400 MHz, CDCl₃): 1.08 (6H, d, J 6.7), 1.39 (9H, s), 3.34 (2H, bm), 3.90 (2H, bm), 4.26 (1H, bm), 6.56 (2H, d, J 8.9), 6.67 (2H, d, J 8.9); MS: 195 [M+H⁺-Boc], 295 [M+H⁺] 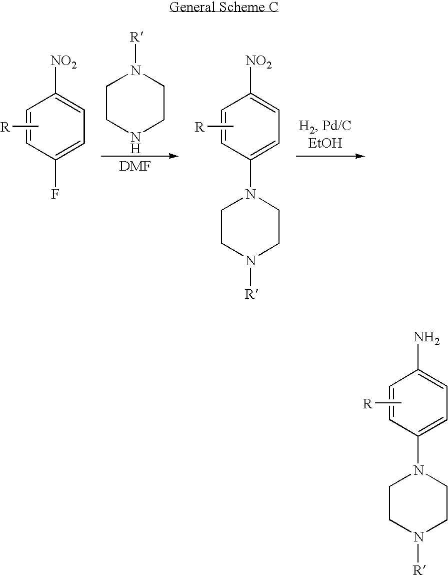
General Method C

### 1-(2-Fluoro-4-nitrophenyl)-4-methylpiperazine

N-Methylpiperazine (30 mL, 27.1 g, 0.268 mol) was cooled in ice/water while adding 3,4-difluoronitrobenzene (2.0 g, 0.0126 mol) with stirring. The mixture was then heated at 100° C. overnight, evaporated to remove all excess N-methylpiperazine and the residue dissolved in 1M hydrochloric acid (30 mL). After washing twice with 20 mL portions of dichloromethane the solution was basified with 5M sodium hydroxide (10 mL). The product was extracted into dichloromethane (twice with 20 mL), dried over sodium sulphate and evaporated giving a yellow oil which solidified on standing. ¹H NMR (CDCl₃) 8.00 (m, 1H) 7.91 (m, 1H) 6.92 (m, 1H) 3.33 (m, 4H) 2.63 (m, 4H) 2.39 (s, 3H).

### 1-(2-Fluoro-4-aminophenyl)-4-methylpiperazine

Obtained by hydrogenation over Pd-10% C of the corresponding nitro compound in ethanol. ¹H NMR (CDCl₃) 6.75 (m, 1H) 6.33 (m, 2H) 3.48 (m, 2H) 2.94 (m, 4H) 2.53 (m, 4H) 2.29 (s, 3H).

Specific Syntheses:

### tert-Butyl 4-(2-difluoromethoxy-4-nitrophenyl)piperazine-1-carboxylate

### 1-(2-Difluoromethoxy-4-nitrophenyl)piperazine

A stirred mixture of 1-bromo-2-difluoromethoxy-4-nitrobenzene (prepared from the corresponding phenol following the procedure outlined in WO9749710A1; 2.68 g, 10 mmol), piperazine (1.12 g, 13 mmol), potassium carbonate (2.07 g, 15 mmol), tetrabutylammonium bromide (0.03 g, 0.1 mmol) and dry dimethyl sulphoxide (20 mL) was heated under nitrogen at 120° C. for 3 h. The product was added to water (100 mL) and 6M hydrochloric acid (10 mL, 60 mmol), washed with ethyl acetate until the washings were colorless and the aqueous layer basified with 5M sodium hydroxide solution (20 mL, 100 mmol). Extraction with ethyl acetate (3× with 50 mL), drying (sodium sulphate) and evaporating gave product as viscous orange oil. ¹H NMR (CDCl₃) 8.00 (m, 1H) 7.92 (m, 1H) 6.93 (m,1H) 6.47 (t, J=73.6, 1H) 3.18 (m, 2H) 2.98 (m, 2H) 2.54 (s, 1H)

### tert-Butyl 4-(2-difluoromethoxy-4-nitrophenyl)piperazine-1-carboxylate

The above product (1.64 g, 6 mmol) was dissolved in dry tetrahydrofuran (25 mL) and di-tert-butyl dicarbonate (1.26 g, 6 mmol) added. After stirring overnight the mixture was evaporated and the resulting orange solid recrystallised from ethyl acetate giving the final product. ¹H NMR (CDCl₃) 8.03 (m, 1H) 7.93 (m, 1H) 6.48 (t, 1H) 3.53 (m, 2H) 3.15 (m, 2H) 1.42 (s, 9H).

### tert-Butyl 4-(2-difluoromethoxy-4-aminophenyl)piperazine-1-carboxylate

Obtained by hydrogenation over Pd-10% C of the corresponding nitro compound in ethanol. ¹H NMR (CDCl₃) 7.73 (m, 1H) 6.56 (t, 1H) 6.42 (m, 2H) 3.46 (m, 2H) 2.80 (m, 2H) 1.40 (s, 9H).

### 4-(4-Amino-2-fluorophenyl)-1,2-dimethylpiperazine

### 4-(2-Fluoro-4-nitrophenyl)-2-methylpiperazine

To rac-2-methylpiperazine (2.64 g, 23.1 mmol) in acetonitrile (50 mL) was added triethylamine (1.95 g, 2.7 mL, 19.2 mmol) followed by 3,4-difluoronitrobenzene (1 g, 7.7 mmol) dropwise over 5 min under a nitrogen atmosphere. The resulting yellow solution was allowed to stir at room temperature for 3 days. Excess acetonitrile was removed by evaporation under reduced pressure and the residue reconstituted in DCM (50 mL), washed with water (2×50 mL), dried (MgSO₄) and concentrated to afford the title compound as a yellow solid. LC-MS (UV 215 nm): 100%; m/z 240.19; 0.91 min. ¹H NMR (CDCl₃): 1.13 (3H, d, J 6.4), 2.56 (1H, dd, J 10.2 11.7), 2.91-2.99 (1H, m), 3.00-3.13 (3H, m), 3.52-3.59 (2H, m), 6.91 (1H, t, J 8.8), 7.85-8.01 (2H, m).

### 1,2-Dimethyl-4-(2-fluoro-4-nitrophenyl)piperazine

To a solution of 4-(2-fluoro-4-nitrophenyl)-2-methylpiperazine (1 g, 4.2 mmol) in formic acid (10 mL) was added paraformaldehyde (1.2 mL, 15.8 mmol of a 36.5% v/v aqueous solution). The resulting yellow solution was heated at 100° C. for 18 h. Upon cooling, excess formic acid/paraformaldehyde was removed under reduced pressure and the residue basified with 1M KOH solution. The resulting yellow precipitate was extracted into DCM (3×25 mL), dried (MgSO₄) and concentrated to afford the title compound as a yellow solid. LC-MS (UV 215 nm): 100%; m/z 254.40; 1.93 min. ¹H NMR (CDCl₃): 1.13 (3H, d, J 6.4), 2.28-2.34 (1H, m), 2.35 (3H, s), 2.43-2.52 (1H, m), 2.73 (1H, dd, J 10.0 12.0), 2.87-2.91 (1H, m), 3.07-3.14 (1H, m), 3.47-3.51 (1H, m), 3.55-3.57 (1H, m), 6.90 (1H, t, J 8.8), 7.84-7.92 (1H, m), 7.96-8.01 (1H, m).

### 4-(4-Amino-2-fluorophenyl)-1,2-dimethylpiperazine

Absolute ethanol (2 mL) was added to a two-necked round bottomed flask containing palladium on carbon (0.09 g, 0.42 mmol). The reaction vessel was evacuated and purged with nitrogen three times. 1,2-Dimethyl-4-(2-fluoro-4-nitrophenyl)piperazine (1.06 g, 4.2 mmol) in absolute ethanol (10 mL) was added and the vessel purged thrice more with nitrogen. After purging thrice with hydrogen, the reaction was left to stir under a hydrogen atmosphere at room temperature for 18 h. The reaction mixture was filtered through a pad of Celite washing with additional ethanol. Excess ethanol was removed under reduced pressure to afford the title compound as an off-white oil. LC-MS (UV 215 nm): 93%; m/z 224.35; 0.58 min. ¹H NMR (DMSO-d₆): 1.08 (3H, d, J 5.8, —CH(CH₃)), 2.37 (3H, s, —NCH₃), 2.43-2.59 (3H, m, —NCH(H)CH₂N—), 2.73-2.83 (1H, m, —NCH(H)), 2.90-3.06 (3H, m, —NCH(H)), 5.01 (2H, br, ArNH₂), 6.25-6.49 (2H, m, ArH), 6.69-6.81 (1H, m, ArH). 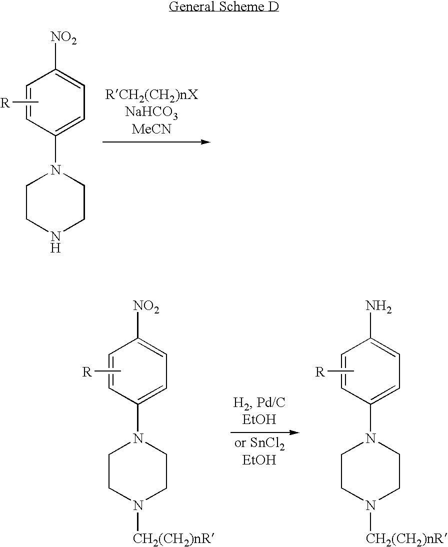
General Method D

### 4-(4-(3-Dimethylaminopropyl)piperazino)nitrobenzene

Prepared according to a slightly modified procedure from U.S. Pat. No. 3,331,845. A mixture of 4-nitrophenylpiperazine (2.1 g, 10 mmol), sodium hydrogen carbonate (2.5 g, 30 mmol), N,N-dimethyl-N-(3-chloropropyl)amine hydrochloride (1.9 g, 12 mmol) in isopropanol (80 mL) was heated at 80° C. for 18 h. The mixture was then allowed to cool, the solid filtered off and the solvent removed under reduced pressure. Ethyl acetate (ca. 200 mL) was added and the residue was washed with saturated brine twice (50 mL each time). The organic layer was dried over sodium sulphate and the solvent evaporated under reduced pressure. The crude compound was purified by column chromatography, eluting with dichloromethane/methanol 9/1 (containing 1% N,N-dimethylethylamine) to give the title compound as a yellow solid. ¹H NMR (CDCl₃, 400 MHz): 1.6 (2H, m); 2.15 (6H, s); 2.25 (2H, m); 2.35 (2H, m); 2.5-2.55 (4H, m); 3.35-3.4 (4H, m); 6.75 (2H, d, J 8 Hz); 8.05 (2H, d, J 8 Hz) MS: 293, 248

### 4-(4-(3-Dimethylaminopropyl)piperazino)aniline

A solution of 4-(4-(3-dimethylaminopropyl)piperazino)nitrobenzene (1.5 g) in methanol (50 mL) was hydrogenated at atmospheric pressure over Pd (5% on carbon) (0.3 g; 50% water content) for 4 h. The catalyst was filtered off and the solvent removed under reduced pressure to give the title compound as a brown solid ¹H NMR (CDCl₃, 400 MHz): 1.65 (2H, m); 2.15 (6H, s); 2.25 (2H, m); 2.35 (2H, m); 2.5-2.55 (4H, m); 2.95-3.05 (4H, m); 6.55 (2H, d, J 7 Hz); 6.75 (2H, d, J 7 Hz) MS: 263, 218

### 4-(4-(3-Chlorobenzyl)piperazino)aniline

4-(4-(3-chlorobenzyl)piperazino)nitrobenzene (3 g, 9 mmol, prepared as in the general method) was dissolved in ethanol (100 mL). Tin (II) chloride dihydrate (10.1 g, 45 mmol) was added and the reaction heated to 80° C. for 66 h. The reaction mixture was concentrated under reduced pressure. A saturated solution (200 mL) of Rochelle's salt (sodium potassium tartrate) was prepared, and solid NaHCO₃ was added to this until no more would dissolve. Ethyl acetate (200 mL) was added to the vessel, followed by the reaction mixture. The solution was then stirred until clear. The phases were separated, and the aqueous layer washed with ethyl acetate (50 mL). The organic layers were combined, washed with saturated brine, dried over magnesium sulphate, and evaporated to give the title compound. ¹H NMR (CDCl₃, 400 MHz): 2.5-2.55 (4H, m); 2.95-3.05 (4H, m); 3.47 (2H, s); 6.5-6.6 (2H, m); 6.7-6.8 (4H, m); 7.15-7.25 (4H,m); MS: 302, 304.

### 4-(4-Aminophenyl)-1-(2-tert-butoxycarbonylaminoethyl)piperazine

### 1-(2-Hydroxyethyl)-4-(4-nitrophenyl)piperazine

1-(4-Nitrophenyl)piperazine (12.0 g, 0.058 mol) and 2-bromoethanol (8.7 g, 0.070 mol) were dissolved in acetonitrile (175 mL) and treated with Hunig's base (9.0 g, 0.070 mol). The mixture was refluxed overnight, then the solvent evaporated and the residue redissolved in dichloromethane. The organic layer was washed with water and brine, dried over Na₂SO₄ and evaporated under reduced pressure to give the title compound.

### 1-(2-Chloroethyl)-4-(4-nitrophenyl)piperazine

1-(2-Hydroxyethyl)-4-(4-nitrophenyl)piperazine (5 g, 0.02 mol) was dissolved in 50 mL of DCM and treated with HCl (40 mL of a 1M solution in Et₂O) under a drying tube for 90 min. The solvent was evaporated, the residue dissolved in thionyl chloride (60 mL) and the mixture refluxed at 80° C. After 5 h, the reaction was complete as shown by LCMS and the thionyl chloride was removed under reduced pressure redissolving in DCM and evaporating three times to give the title compound as the HCl salt. LCMS: 93%, t=0.88 min, [MH+]=270.23

### 1-(2-Azidoethyl)-4-(4-nitrophenyl)piperazine

1-(2-Chloroethyl)-4-(4-nitrophenyl)piperazine (1 g, 0.0033 mol) was dissolved in dimethylsulphoxide (30 mL) and sodium azide (0.34 g, 0.0052 mol) was added followed by Hunig's base (0.84 g, 0.0065 mol). The mixture was stirred at 80° C. overnight in a sealed tube, then diluted with ethyl acetate and washed with water, dried and evaporated to yield the title compound. LCMS: 90%, t=0.85 min, [MH+]=277.26

### 1-(2-Tert-butoxycarbonylaminoethyl)-4-(4-nitrophenyl)piperazine

1-(2-Azidoethyl)-4-(4-nitrophenyl)piperazine (0.8 g, 0.0029 mol) was dissolved in anhydrous ethyl ether (35 mL) under nitrogen, and tributyl phosphine (0.76 mL, 0.0032 mol) was added dropwise. The mixture was stirred at room temperature for 1 h then cooled to −50° C. before adding di-tert-butyldicarbonate (0.63 g, 0.0032 mol dissolved in little ether) and stirring for a further hour. Saturated sodium hydrogen carbonate (2 mL) was added and the flask was allowed to warm to room temperature. Further 20 mL of saturated sodium hydrogen carbonate and 20 mL ethyl acetate were added, the organic phase separated and the aqueous layer extracted with ethyl acetate. The organic layers were combined, dried and evaporated. The crude material was purified on silica (2% MeOH:DCM) to give the title compound. LCMS: 91%, t=1.25 min, [M-56]+=295.32

### 4-(4-Aminophenyl)-1-(2-tert-butoxycarbonylaminoethyl)piperazine

1-(2-Tert-butoxycarbonylaminoethyl)-4-(4-nitrophenyl)piperazine (0.8 g, 0.0023 mol) was dissolved in ethanol (40 mL) and stirred with Pd/C (10%, 100 mg) under a hydrogen atmosphere overnight. The catalyst was filtered off and solvent evaporated to yield the title compound. LCMS: 90%, t=0.82 min. ¹H NMR (CDCl₃) 6.85 (2H, d); 6.65 (2H, d); 5.0 (1H, s); 3.3 (2H, t); 3.05 (4H, m); 2.6 (4H, m); 2.5 (2H, t); 1.5 (9H, s).

### 4-(4-Aminophenyl)-1-((2-N-tert-butoxycarbonylethylamino)ethyl)piperazine

### 1-((2-Ethylamino)ethyl)-4-(4-nitrophenyl)piperazine

1-(2-Chloroethyl)-4-(4-nitrophenyl)piperazine (2.5 g, 0.0082 mol), ethylamine (20 mL, 0.041 mol) and Hunig's base (2.1 g, 0.016 mol) were dissolved in ethanol (150 mL) and stirred at 80° C. in a pressure tube for 48 h. The solvent evaporated and the residue dissolved in DCM; the organic layer was washed with brine to form a precipitate which was collected and combined with the material recovered from evaporation of the organic layer to give the title compound as the HCl salt LCMS: 86%, t=0.92 min, [MH+]=270.23.

### 1-((2-N-Tert-butoxycarbonylethylamino)ethyl)-4-(4-nitrophenyl)piperazine

1-((2-Ethylamino)ethyl)-4-(4-nitrophenyl)piperazine (1.7 g, 0.0061 mol) was dissolved in dioxane/water 1/1 (70 mL) and di-tert-butyldicarbonate (1.47 g, 0.0067 mol) added. The reaction was stirred overnight at room temperature under nitrogen, the organic solvent was evaporated and the aqueous layer extracted into DCM, dried over Na₂SO₄ and evaporated. The residue was purified on silica eluting with 5% MeOH:DCM to give the title compound. LCMS: 87%, t=1.20 min, [MH+]=379.35, [M-56]+=323.31.

### 4-(4-Aminophenyl)-1-((2-N-tert-butoxycarbonylethylamino)ethyl)piperazine

1-((2-N-tert-Butoxycarbonylethylamino)ethyl)-4-(4-nitrophenyl)piperazine (1.0 g, 0.0027 mol) was hydrogenated overnight over Pd—C (10%, 100 mg) in ethanol (50 mL) to yield the the title aniline. LCMS: 94%, t=1.10 min, [MH+]=349.41, [M-56]+=293.35. ¹H NMR (CDCl₃) 6.8 (2H, d); 6.65 (2H, d); 5 (1H, s); 3.35 (2H, t); 3.25 (2H, t); 3.05 (4H, m); 2.65 (4H, t); 2.5 (2H, t); 1.45 (9H, s); 1.15 (3H, t). 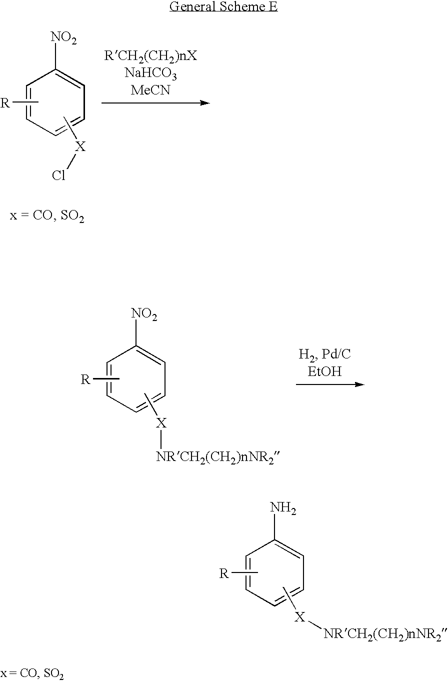
General Method E

### N-(2-Dimethylaminoethyl)-3-nitrobenzamide

3-Nitrobenzoyl chloride (2 g, 10.77 mmol) was loaded into a round bottomed flask, placed under a N₂ atmosphere and dissolved in anhydrous dichloromethane (10 mL). The mixture was cooled to 0° C. and N,N-dimethylethylenediamine (0.98 mL, 8.98 mmol) was added to the reaction. The reaction was allowed to warm to room temperature and left to stir for 18 h. After 18 h the reaction had given a precipitate which was isolated by filtration and washed with dichloromethane to give 2.28 g of a white solid, which was partitioned between dichloromethane and a saturated aqueous NaHCO₃ solution. The aqueous layer was separated and extracted dichloromethane. The organic layers were combined, dried over Na₂SO₄ and the solvent was removed under reduced pressure to afford the title compound as a yellow solid. MS: 193, 238; ¹H NMR (400 MHz, dmso-d6): 2.19 (6H, s), 2.42 (2H, t, J 6.8 Hz), 3.39 (2H, q, J 12.4 Hz, 6.7 Hz), 7.78 (1H, t, J 7.9 Hz), 8.29 (1H, ddd, J 7.9 1.8, 1.1 Hz), 8.38 (1H, ddd, J 8.1 Hz, 2.3, 1.0 Hz), 8.68 (1H, t, J 1.8 Hz), 8.81 (1H, t, J 5.7 Hz).

### 3-(N-(2-Dimethylaminoethylcarbamoyl))aniline

Palladium on carbon (200 mg, 10% w/w) was loaded to a three-necked flask and ethanol (1 mL) was added. This was then fitted with a three-way tap with balloon. The flask was then placed under vacuum then purged with nitrogen, this was repeated twice more. The amide (2.0 g, 8.4 mmol) was dissolved in ethanol (20 mL), this was then added to the reaction. The reaction was then placed under vacuum and purged with nitrogen three more times. It was then placed under vacuum again then purged with hydrogen, this was repeated once more leaving the balloon filled with hydrogen. The reaction was left at room temperature overnight under a hydrogen atmosphere. The reaction solution was then filtered through a celite plug washing with ethanol. The filtrates were combined and solvent removed to give a clear colorless oil. MS: 208; ¹H NMR (400 MHz, CDCl₃): 2.22 (6H, s), 2.27 (2H, t, J 5.9 Hz), 3.45 (2H, q, J 11.6, 5.3 Hz), 6.71 (1H, ddd, J 7.9, 2.4, 1.0 Hz), 6.85 (1H, bs), 7.0-7.15 (3H, m) 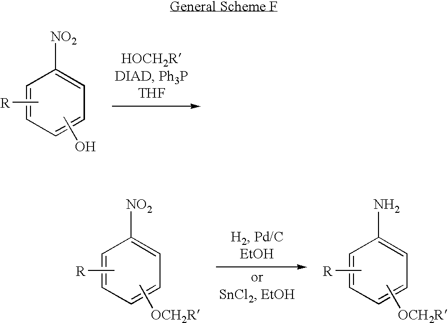
General Method F

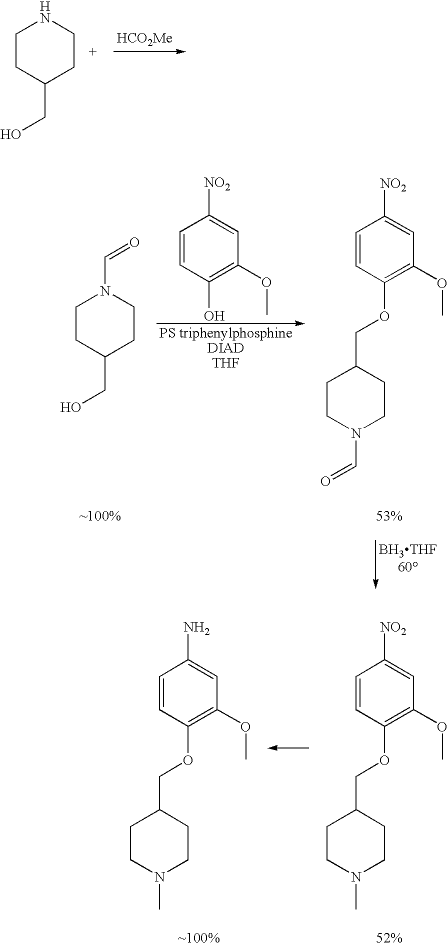

### N-Formyl-4-piperidinemethanol

4-Piperidinemethanol (10 g, 87 mmol) was dissolved in methyl formate (7 mL, 113 mmol) 0° C., and maintained at that temperature for 30 min, then allowed to reach 20° C. and stirred 90 min. Solid sodium hydroxide was added (0.87 g, pellets) and the mixture was left overnight. Dichloromethane was added, the NaOH removed by filtration and the solution treated with 1M HCl in ether (10 mL). The mixture was filtered through Celite and the solvent was removed under reduced pressure to afford the crude title compound. ¹H NMR (400 MHz, CDCl₃): 0.85-1.1 (2H, m); 1.55-1.85 (3H, m); 2.5-2.7 (1H, m); 2.95-3.1 (1H, m); 3.3 (2H, d, J 7 Hz); 3.6-3.7 (1H, m); 4.1-4.3 (1H, m); 8 (1H, s)

### N-Formyl-4-(2-methoxy-4-nitrophenoxymethyl)piperidine

4-Nitroguaiacol (2 g, 11.8 mmol), N-formyl 4-piperidinemethanol (1.13 g, 7.89 mmol) and polymer-supported triphenylphosphine (3 mmol/g, 3.94 g, 11.8 mmol) were dissolved in tetrahydrofuran (30 mL). The mixture was cooled to 0° C. and diisopropyl azodicarboxylate (2.33 mL, 11.8 mmol) was added dropwise. The mixure was stirred at 0° C. for 30 min then at 20° C. overnight. The resin was filtered off, washed with dichloromethane then methanol and the filtrate evaporated to give a deep orange oil. The oil was taken up in dichloromethane, washed with 2M NaOH, 2M HCl then brine, dried and evaporated giving a pale brown oil. This was taken up in 50:50 ethyl acetate:hexane, filtered through celite, filtrate evaporated, taken up in ethyl acetate and washed further with 1M NaOH. The organic layer was separated, dried over Na₂SO₄, the solvent removed under reduced pressure and the residue columned in 50:50 ethyl acetate:hexane to remove impurities. The product was then eluted with 9:1 dichloromethane:methanol to give a yellow oil, which crystallised on cooling. ¹H NMR (400 MHz, CDCl₃): 1.15-1.3 (2H, m); 1.85-1.9 (1H, m); 2.6-2.7 (1H, m); 3-3.1 (1H, m); 3.7-3.8 (1H, m); 4.0 (2H, d, J 7 Hz); 4.15-4.25 (1H, m); 7.2 (1H, d, J 8 Hz); 7.75 (1H, d, J 2 Hz); 7.9 (1H, dd, J 2 and 8 Hz); 8 (1H, s)

### N-Methyl-4-(2-methoxy-4-nitrophenoxymethyl)piperidine

A suspension of N-formyl-4-(2-methoxy-4-nitrophenoxymethyl)piperidine (1.24 g, 4.2 mmol) in tetrahydrofuran (5 mL) under nitrogen was stirred while adding the borane solution (8.4 mL of a 1M soln in THF) then heated to 60° C. for 2 h. Further borane solution (to a total of 5 equivalents) and 20 mL tetrahydrofuran (20 mL) were added and the mixture was heated overnight. The mixture was cooled, methanol (25 mL) was added carefully followed by dichloromethane. The mixture was then washed with brine, 2M NaOH, dried over Na₂SO₄ and solvent evaporated. The residue was dissolved in methanol, a few drops of acetic acid added and the mixture was heated under reflux for 3 days. Evaporation of the solvent and chromatography in 9:1 dichloromethane:methanol containing 1% triethylamine afforded the product as a brown solid. ¹H NMR (400 MHz, dmso-d₆): 1.4-1.5 (2H, m); 1.85-2 (3H, m); 2-2.1 (2H, m); 2.8-3 (2H, m); 4.05 (3H, s); 4.15 (2H, d, J 7 Hz); 7.35 (1H, d, J 8 Hz); 7.9 (1H, d, J 2 Hz); 8.05 (1H, dd, J 2 and 8 Hz)

### N-Methyl-4-(2-methoxy-4-nitrophenoxymethyl)piperidine

Catalytic reduction over Pd (10% C) in EtOH gave the aniline as a red-brown solid. ¹H NMR (400 MHz, CDCl₃): 1.3-1.5 (2H, m); 1.7-1.9 (3H, m); 2-2.1 (2H, m); 2.9-3 (2H, m); 3.4 (2H, broad s); 3.7 (2H, d, J 7 Hz); 3.75 (3H, s); 6.15 (1H, dd, J 1 and 7 Hz); 6.25 (1H, d, J 1 Hz); 6.65 (1H, d, J 7 Hz). 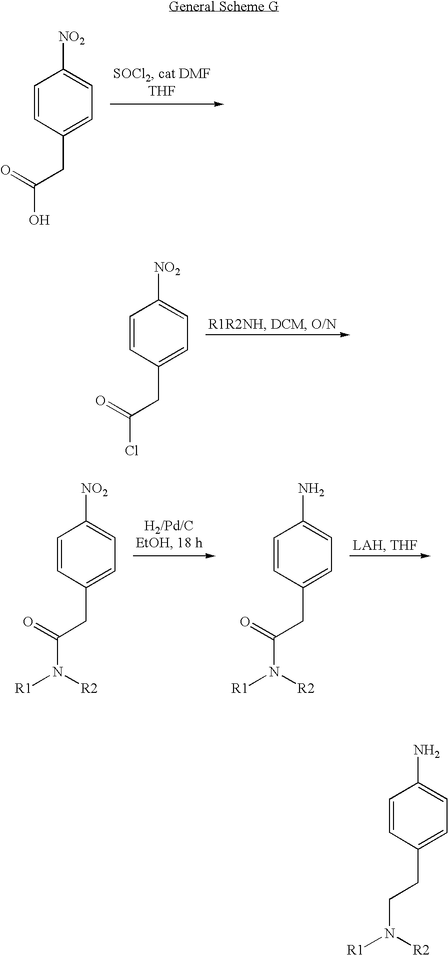
General Method G

### 1-Methyl-4-[(4-nitrophenyl)acetyl]piperazine

4-Nitrophenylacetic acid (2.00 g, 0.011 mol) was dissolved in anhydrous THF (20 mL) with gradual addition of thionyl chloride (1.03 mL, 0.0143 mol) and a catalytic amount of DMF (2 drops) at room temperature and stirred for 24 h. On completion, the reaction was quenched in situ with N-methylpiperazine (3.85 g, 0.038 mol) added dropwise in a solution of DCM (20 mL) at room temperature and stirred overnight to give a beige suspension. The solvent was removed in vacuo and the residue partitioned between DCM (30 mL) and sodium hydroxide (1N, 30 mL). The organic layer was washed twice, dried over sodium sulphate and filtered. Removal of the solvent in vacuo the title compound as an amber oil, which solidified on standing. LCMS:2.5 min; Rt 0.82 (m/z 264, M+H⁺) 98% ¹H NMR: (400 MHz, CDCl₃); 2.28 (3H, s), 2.32 (2H, dd, J 5.03, 5.08), 2.39 (2H, t, J 5.13), 3.49 (2H, t, J 5.08), 3.67 (2H, t, J 5.03), 3.82 (2H, s), 7.42 (2H, d, J 8.78), 8.19 (2H, d, J 8.78)

### 4-(4-Methylpiperazin-1-yl)carbonylmethylaniline

A solution of 1-methyl-4-[(4-nitrophenyl)acetyl]piperazine(1.5 g, 5.70 mmol) in ethanol (30 mL) was reduced over palladium/charcoal (10% wt, 50% wet, 150 mg) with hydrogen under atmospheric pressure and room temperature for 18 h. The catalyst was separated by filtration through celite and the solvent evaporated to the title compund as a brown oil. ¹H NMR: (400 MHz, CDCl₃); 2.21 (2H, t, J 5.01), 2.24 (3H, s), 2.34(2H, t, J 5.13), 3.45 (2H, t, J 5.13), 3.61 (2H, s), 3.63-3.66 (2H, m), 6.64 (2H, d, J 8.56), 7.01 (2H, d, J 8.31).

### 4-[2-(4-Methyl-piperazin-1-yl)ethyl]aniline

4-(4-Methylpiperazin-1-yl)carbonylmethylaniline (596 mg, 2.55 mmol) was treated in anhydrous THF (20 mL) under nitrogen with lithium aluminium hydride (291 mg, 2.67 mmol) overnight. The reaction was quenched with water (3×0.29 mL), 15% sodium hydroxide (3×0.29 mL) and again water (3×0.29 mL). The resulting precipitate was removed by filtration. Evaporation of the filtrate afforded the title compound as an orange oil. LC:2.5 min; Rt 0.21(m/z 220, M+H⁺) 90%, ¹H NMR: (400 MHz, CDCl₃); 2.30 (3H, s), 2.40-2.73 (2H+8H, m), 2.69 (2H, m); 6.62 (2H, d, J 8.31), 6.99 (2H, d, J 8.31). 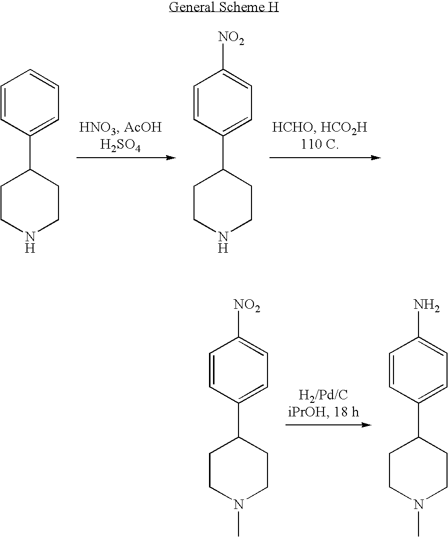
General Method H

### 4-(4-Nitrophenyl)piperidine

4-Phenylpiperidine (8 g, 49 mmol) was dissolved in 40 mL acetic acid and stirred with cooling below 25° C. while adding a solution of 2.64 mL sulphuric acid in 40 mL acetic acid. The solution was stirred at 20° C. while adding a solution of 2.08 mL 99% nitric acid in 20 mL acetic acid. Sulphuric acid (40 mL) was added without cooling, the temperature peaking at 58° C. When the solution had cooled to 25° C. it was added to 100 g ice/water and basified with a total of 150 g sodium hydrogen carbonate at 40° C. The mixture was then brought to pH 14 with 5M—sodium hydroxide solution. The mixture was extracted with dichloromethane (3×150 mL), dried with sodium sulphate and evaporated giving a pale yellow solid. Recrystallization from a total of 180 mL cyclohexane (hot filtration) gave the product as pale beige solid. NMR δ 8.18(d,2H), 7.40(d,2H), 3.21(m,2H), 2.77(m,3H), 1.82(m,3H), 1.68(m,2H).

### 1-Methyl-4-(4-nitrophenyl)piperidine

4-(4-Nitrophenyl)piperidine (515 mg) was added to 4 mL 90% formic acid and 1.5 mL formalin added. The solution was stirred and heated at 110° C. for 17 h, evaporated and the solids dissolved in 20 mL water. After basification to pH 14 with 5M sodium hydroxide solution the precipitated solid was extracted into t-butyl methyl ether (3×30 mL). Drying (sodium sulphate) and evaporating gave pure product as pale cream solid. NMR δ 8.15(d,2H), 7.39(d, 2H), 3.00(m,2H), 2.60(m,1H), 2.31(s, 3H), 2.08 (m, 2H), 1.86 (m, 4H).

### 4-(4-Aminophenyl)-1-methylpiperidine

Hydrogenation of the above compound in 30 mL isopropanol over 100 mg of 10% palladium on charcoal for 5 h, filtration and evaporation gave the pure product as cream solid. NMR δ 7.00(d, 2H), 6.63(d, 2H), 3.57(s, 2H), 2.98(m, 2H), 2.38(m, 1H), 2.30(s, 3H), 2.02(m, 2H),1.78(m, 4H). 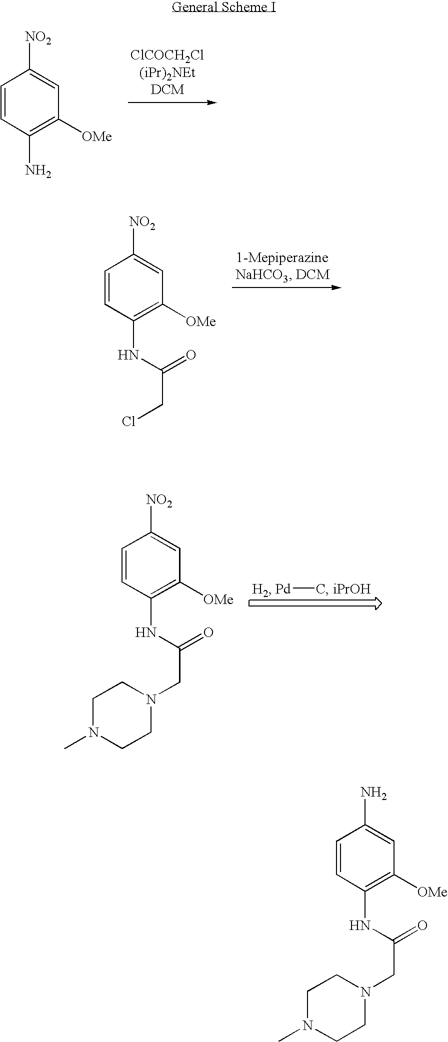
General Method I

### N-(2-methoxy-4-nitrophenyl)chloroacetamide

2-Methoxy-4-nitroaniline (8.40 g, 50 mmol) was dissolved in 200 mL dichloromethane and stirred at 0° C. while adding diisopropylethylamine (6.45 g, 50 mmol) then a solution of chloroacetyl chloride (5.65 g, 50 mmol) in 50 mL dichloromethane. The mixture was stirred for 17 h at 20-25° C., evaporated and 300 mL ethyl acetate added. The solution was washed with 2×100 mL 2M hydrochloric acid then brine, dried (sodium sulphate) and evaporated. The residue dissolved in 200 mL warm toluene, treated with charcoal, filtered, and then chromatographed on 300 mL flash silica in a 10 cm sinter funnel. Elution with toluene and evaporation afforded a bright yellow oil which solidified on standing. NMR showed an 11:4 mixture of product and starting aniline—product δ 9.17(s, 1H), 8.56(m,1H), 7.94(m, 1H), 7.79(m, 1H), 4.22(s, 2H), 4.03(s, 3H). Used without further purification.

### 1-[(2-Methoxy-4-nitrophenyl)carbamoyl]methyl-4-methylpiperazine

The above product (3.06 g, containing 10 mmol of the chloroacetamide) was dissolved in 20 mL dichloromethane and 100 mL isopropanol. 1-Methylpiperazine (1.00 g, 10 mmol) was added followed by 4.20 g sodium hydrogen carbonate (50 mmol). The mixture was stirred at 20° C. for 4 h then left overnight.

Next day the mixture was heated under reflux for 4 h, evaporated and the residue treated with 200 mL water/200 mL ethyl acetate. Further ethyl acetate was added to dissolve all the product. The ethyl acetate extracts were washed well with water then with 2M citric acid solution (3×40 mL). The acid extracts were washed with ethyl acetate (2×50 mL) then brought to pH 12 with 5M sodium hydroxide solution. The solid was collected and washed with water then dried in air giving the product as pale yellow needles. NMR δ 10.18(s, 1H), 8.59(m, 1H), 7.93(m, 1H), 7.78(m, 1H), 4.01(s, 3H), 3.20(s, 2H), 2.40-2.80(m, 8H), 2.50(s, 3H).

### 1-[(2-Methoxy-4-aminophenyl)carbamoyl]methyl-4-methylpiperazine

The above product was dissolved in 120 mL isopropanol, 300 mg 10% palladium on charcoal added and stirred under hydrogen for 6 h. Filtration and evaporation gave the product as cream solid. NMR δ 9.47(s, 1H), 8.09(m, 1H), 6.30(m, 2H), 3.83(s, 3H), 3.58(s 2H), 3.11(s, 2H), 2.30-2.70(m, 8H), 2.30(s, 3H). 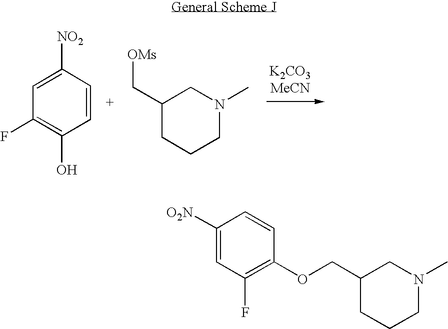
General Method J

### Preparation of 3-(2-Fluoro-4-nitrophenoxymethyl)-1-methylpiperidine

3-Hydroxymethyl-1-methylpiperidine (2 g, 9.66 mmol) was dissolved in DCM (15 mL), the solution was cooled to 0° C. and methanesulfonyl chloride (0.9 mL, 11.6 mmol) was added dropwise. The reaction was then allowed to warm up to room temperature and left stirring for 24 h. After this time water (20 mL) was added to the reaction and the organic layer was separated, dried over Na₂SO₄, filtered and solvent removed to give a colorless oil. ¹H NMR indicated a mixture of the starting piperidine and the desired product. This was then dissolved in acetonitrile (30 mL) along with 2-fluoro-4-nitrophenol (1.52 g., 9.66 mmol); potassium carbonate (2.67 g., 19.32 mmol) was added and the reaction was heated at 90° C. for 18 h, then allowed to cool to room temperature. The solids were filtered and solvent removed from the filtrate. This gave 3.5 g of an orange oil. Column chromatography (5% MeOH/DCM) gave a yellow solid which was shown to be a mixture of 2-fluoro-4-nitrophenol and 3-(2-fluoro-4-nitrophenoxymethyl)-1-methylpiperidine. The material was partitioned between DCM (30 mL) and saturated potassium carbonate solution (30 mL). The DCM layer was removed and washed with 5M HCl (20 mL). The acidic layer was then basified to pH 10 and extracted twice with DCM (30 mL). The organics combined, dried over Na₂SO₄, filtered and solvent removed to give 3-(2-fluoro-4-nitrophenoxymethyl)-1-methylpiperidine as a yellow oil. ¹NMR (400 MHz, (CD₃OD) 8.16-8.06 (2 H, m), 7.33 (1 H, t, J 8.8), 4.17 (1 H, m), 4.09 (1 H, m), 3.32 (1 H, d, J 10.8), 3.02 (1 H, t, J 11.5), 2.47 (3 H, s), 2.22 (3 H, m), 1.89 (3 H, m), 1.75 (1 H, m), 1.28 (1 H, m).

Alternatively, the nitro derivative was prepared as follows: 3,4-Difluoronitro phenol (3 g, 18.7 mmol) and 3-hydroxy-1-methylpiperidine (2.5 g, 19.3 mmol) were dissolved in dry THF (100 mL) under nitrogen. Sodium hydride (60%, 1 g, 25 mmol) was slowly added under positive nitrogen pressure. The resulting light yellow solution was heated at 60° C. for 2.5 h. The dark-red solution was left to cool to room temperature and quenched with a solution of acetic acid (0.3 mL, 5.2 mmol) in methanol (10 mL). Solvent was removed under reduced pressure to yield an orange solid that was purified by column chromatography using DCM: MeOH (75: 25) as eluent to yield 3-(2-fluoro-4-nitro phenoxymethyl)-1-methylpiperidine as an orange oil.

Catalytic hydrogenation as in general Method A afforded the aniline derivative.

Further examples of anilines include the following (NMR spectra at 400 MHz, in CDCl₃ unless otherwise stated):

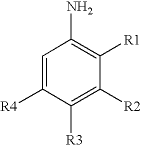

| Ex. | Method | R1 | R2 | R3 | R4 | MS | NMR |
|---|---|---|---|---|---|---|---|
| 1 | A (chloro- | H | H | 3-(di- | H | 181 | 2.25 (6H, s); |
| | alkyl phenol | | | methyl- | | | 2.65 (2H, t, J 7 |
| | displacement) | | | amino)- | | | Hz); 3.9 (2H, |
| | | | | ethoxy | | | t, J 7 Hz); 6.5- |
| | | | | | | | 7 (2H, m); |
| | | | | | | | 6.65-6.75 |
| | | | | | | | (2H, m) |
| 2 | A (chloro- | H | H | 3-(di- | | | See specific |
| | alkyl phenol | | | methyl- | | | example |
| | displacement) | | | amino)- | | | |
| | | | | propoxy | | | |
| 3 | A (chloro- | H | OCH₃ | 2-((4- | OCH₃ | 296 | 2.25 (3H, s); |
| | alkyl phenol | | | CH₃)pip- | | | 2.4-2.7 (8H, |
| | displacement) | | | erazin-1- | | | m); 2.75 (2H, |
| | | | | yl)ethoxy | | | t, J 7 Hz); 3.7 |
| | | | | | | | (6H, s); 3.9 |
| | | | | | | | (2H, t, J 7 Hz); |
| | | | | | | | 5.9 (2H, s) |
| 4 | A (chloro- | H | OCH₃ | 3-((4- | OCH₃ | 310 | 1.8-1.9 (2H, |
| | alkyl phenol | | | CH₃)pip- | | | m); 2.2 (3H, |
| | displacement) | | | erazin-1- | | | s); 2.3-2.6 (10 |
| | | | | yl)propoxy | | | H, m); 3.7 |
| | | | | | | | (6H, s); 3.85 |
| | | | | | | | (2H, t, J 7 Hz); |
| | | | | | | | 5.9 (2H, s) |
| 5 | A (chloro- | H | OCH₃ | 2-((4- | H | 266 | 2.35 (3H, s); |
| | alkyl phenol | | | CH₃)pip- | | | 2.55-2.8 (10H, |
| | displacement) | | | erazin-1- | | | m); 3.7 (3H, |
| | | | | yl)ethoxy | | | s); 4 (2H, t, J |
| | | | | | | | 7 Hz); 6.1 (1H, |
| | | | | | | | dd, J 2 and 8 |
| | | | | | | | Hz); 6.2 (1H, |
| | | | | | | | d, J 2 Hz); 7.7 |
| | | | | | | | (1H, d, J 8 Hz) |
| 6 | A (chloro- | H | OCH₃ | 3-((4- | H | 280 | 1.9-2.1 (2H, |
| | alkyl phenol | | | CH₃)pip- | | | m); 2.35 (3H, |
| | displacement) | | | erazin-1- | | | s); 2.4-2.6 |
| | | | | yl)propoxy | | | (10H, m); 3.8 |
| | | | | | | | (3H, s); 4 (2H, |
| | | | | | | | t,J 7 Hz); 6.2 |
| | | | | | | | (1H, dd, J 2 |
| | | | | | | | and 8 Hz); 6.3 |
| | | | | | | | (1H, d, J 2 |
| | | | | | | | Hz); 6.8 (1H, |
| | | | | | | | d, J 8 Hz) |
| 7 | A (chloro- | H | OCH₃ | OCH₃ | 2-((4- | 296 | 2.2 (3H, s); |
| | alkyl phenol | | | | CH₃)pip- | | 2.3-2.5 (4H, |
| | displacement) | | | | erazin-1- | | m); 2.5-2.7 |
| | | | | | yl)ethoxy | | (4H, m); 2.8 |
| | | | | | | | (2H, t, J 7 Hz); |
| | | | | | | | 3.65 (3H, s); |
| | | | | | | | 3.75 (3H, s); 4 |
| | | | | | | | (2H, t, J 7 Hz); |
| | | | | | | | 5.8-5.85 (2H, m) |
| 8 | A (chloro- | H | OCH₃ | OCH₃ | 3-((4- | 310 | 1.85-1.95 (2H, |
| | alkyl phenol | | | | CH₃)pip- | | m); 2.2 (3H, |
| | displacement) | | | | erazin-1- | | s); 2.3-2.5 |
| | | | | | yl)propoxy | | (10H, m); 3.65 |
| | | | | | | | (3H, s); 3.75 |
| | | | | | | | (3H, s); 3.95 |
| | | | | | | | (2H, t, J 7 Hz); |
| | | | | | | | 6.85-6.9 (2H, m) |
| 9 | A (chloro- | H | OCH₃ | 2-(piper- | OCH₃ | 281 | 1.3-1.4 (2H, |
| | alkyl phenol | | | idino)- | | | m); 1.5-1.6 |
| | displacement) | | | ethoxy | | | (4H, m); 2.45- |
| | | | | | | | 2.6 (4H, m), |
| | | | | | | | 2.75 (2H, t, J 7 |
| | | | | | | | Hz); 3.65 (6H, |
| | | | | | | | s); 3.95 (2H, t, |
| | | | | | | | J 7 Hz); 5.85 |
| | | | | | | | (2H, s) |
| 10 | A (phenol | H | H | 2- | H | 223 | 2.45-2.55 (4H, |
| | alkylation) | | | (morpholino) | | | m); 2.7 (2H, t, |
| | | | | ethoxy | | | J 7 Hz); 3.65- |
| | | | | | | | 3.7 (4H, m), |
| | | | | | | | 3.95 (2H, t, J 7 |
| | | | | | | | Hz); 6.5-6.6 |
| | | | | | | | (2H, m); 6.65- |
| | | | | | | | 6.7 (2H, m) |
| 11 | A (chloro- | H | OCH₃ | 2- | OCH₃ | 283 | 2.5-2.55 (4H, |
| | alkyl phenol | | | (morpholino) | | | m); 2.7 (2H, t, |
| | displacement) | | | ethoxy | | | J 7 Hz); 3.6- |
| | | | | | | | 3.7 (4H, m); |
| | | | | | | | 3.7 (6H, s); |
| | | | | | | | 4.95 (2H, t, J 7 |
| | | | | | | | Hz); 5.8 (2H, s) |
| 12 | A (chloro- | H | H | (S)-((1- | H | 207 | (dmso-d₆) 1.5- |
| | alkyl phenol | | | CH₃)pyrr | | | 1.6 (1H, m); |
| | displacement) | | | olidin-2- | | | 1.6-1.65 (2H, |
| | | | | yl)methoxy | | | m); 1.9-2 (1H, |
| | | | | | | | m); 2.15-2.25 |
| | | | | | | | (1H, m); 2.35 |
| | | | | | | | (3H, s); 2.5- |
| | | | | | | | 2.6 (1H, m); |
| | | | | | | | 2.9-3 (1H, m); |
| | | | | | | | 4.65-4.7 (1H, |
| | | | | | | | m); 4.7-4.75 |
| | | | | | | | (1H, m); 6.45- |
| | | | | | | | 6.5 (2H, m); |
| | | | | | | | 6.6-6.65 (2H, m) |
| 13 | A (chloro- | H | F | 3-((4- | H | 268 | 1.8-1.9 (2H, |
| | alkyl phenol | | | CH₃)pip- | | | m); 2.2 (3H, |
| | displacement) | | | erazin-1- | | | s); 2.3-2.55 |
| | | | | yl)propoxy | | | (10H, m); 3.9 |
| | | | | | | | (2H, t, J 7 Hz); |
| | | | | | | | 6.3 (1H, m); |
| | | | | | | | 6.4 (1H, m); |
| | | | | | | | 6.7 (1H, m) |
| 14 | A (chloro- | H | F | 3-(piper- | H | 253 | 1.3-1.4 (2H, |
| | alkyl phenol | | | idino)pro | | | m); 1.4-1.5 |
| | displacement) | | | poxy | | | (4H, m); 1.7- |
| | | | | | | | 1.8 (2H, m); |
| | | | | | | | 2.25-2.4 (6H, |
| | | | | | | | m); 3.9 (2H, t, |
| | | | | | | | J 7 Hz); 6.25- |
| | | | | | | | 6.3 (1H, m); |
| | | | | | | | 6.35-6.4 (1H, |
| | | | | | | | m); 6.75-6.85 |
| | | | | | | | (1H, m) |
| 15 | A (chloro- | H | F | 3-(di- | H | 241 | 1.05 (6H, t, J 7 |
| | alkyl phenol | | | ethyl- | | | Hz); 1.9-2 |
| | displacement) | | | amino)- | | | (2H, m); 2.5- |
| | | | | propoxy | | | 2.7 (6H, m), 4 |
| | | | | | | | (2H, t, J 7 Hz); |
| | | | | | | | 6.35-6.4 (1H, |
| | | | | | | | m); 6.4-6.45 |
| | | | | | | | (1H, m); 6.8- |
| | | | | | | | 6.9 (1H, m) |
| 16 | A (chloro- | H | F | 2-((4- | H | 254 | 2.2 (3H, s); |
| | alkyl phenol | | | CH₃)- | | | 2.3-2.4 (4H, |
| | displacement) | | | piperazin-1- | | | m); 2.4-2.65 |
| | | | | yl)ethoxy | | | (4H, m), 2.75 |
| | | | | | | | (2H, t, J 7 Hz); |
| | | | | | | | 4 (2H, t, J 7 |
| | | | | | | | Hz); 6.25-6.3 |
| | | | | | | | (1H, m); 6.3- |
| | | | | | | | 6.35 (1H, m); |
| | | | | | | | 6.75-6.85(1H, m) |
| 17 | A (chloro- | H | 3-(piper- | H | H | 235 | 1.3-1.4 (2H, |
| | alkyl phenol | | idino)pro | | | | m); 1.45-1.55 |
| | displacement) | | poxy | | | | (4H, m); 2.3- |
| | | | | | | | 2.5 (6H, m); |
| | | | | | | | 3.9 (2H, t, J 7 |
| | | | | | | | Hz); 6.1-6.3 |
| | | | | | | | (3H, m); 6.9-7 |
| | | | | | | | (1H, m) |
| 18 | A (chloro- | H | 3-((4- | H | H | 250 | 1.9-2 (2H, m); |
| | alkyl phenol | | CH₃)- | | | | 2.3 (3H, s); |
| | displacement) | | piperazin-1- | | | | 2.4-2.7 (10H, |
| | | | yl)propoxy | | | | m); 4 (2H, m); |
| | | | | | | | 6.2-6.4 (3H, m); |
| | | | | | | | 7-7.1 (1H, m) |
| 19 | F (Mitsu- | H | OCH₃ | (R)- | 323, 223 | | (as N-Boc |
| | nobu) | | | (pyrrolidin- | (as N- | | protected); |
| | | | | 2-yl)- | Boc pro- | | (dmso-d₆): 1.4 |
| | | | | methoxy | tected) | | (9H, broad s); |
| | | | | | | | 1.7-1.8 (2H, |
| | | | | | | | m); 1.8-2 (3H, |
| | | | | | | | m); 3.2-3.25 |
| | | | | | | | (2H, m); 3.65 |
| | | | | | | | (3H, s); 3.75- |
| | | | | | | | 3.85 (2H, m); |
| | | | | | | | 4.7-4.8 (2H, |
| | | | | | | | broad s); 6 |
| | | | | | | | (1H, dd, J 2 |
| | | | | | | | and 8 Hz); |
| | | | | | | | 6.25 (1H, d, J |
| | | | | | | | 2 Hz); 6.65 |
| | | | | | | | (1H, d, J 8 Hz) |
| 20 | A (chloro- | H | Cl | 2-(piper- | H | 255 | 1.3-1.4 (2H, |
| | alkyl phenol | | | idino) | | 257 | m); 1.5-1.6 |
| | displacement) | | | ethoxy | | | (4H, m); 2.4- |
| | | | | | | | 2.6 (4H, m); |
| | | | | | | | 2.8 (2H, t, J 7 |
| | | | | | | | Hz); 4.1 (2H, |
| | | | | | | | t,J 7 Hz); 6.55 |
| | | | | | | | (1H, dd, J 2 |
| | | | | | | | and 8 Hz); 7.7 |
| | | | | | | | (1H, d, J 2 |
| | | | | | | | Hz); 7.8 (1H, |
| | | | | | | | d, J 8 Hz) |
| 21 | A (chloro- | H | F | 2-((4-iso- | H | 282 | 1.05 (6H, d, J |
| | alkyl phenol | | | propyl) | | | 7 Hz); 2.5-2.7 |
| | displacement) | | | piperazin-1- | | | (9H, m); 2.8 |
| | | | | yl)-ethoxy | | | (2H, t, J 7 Hz), |
| | | | | | | | 4.1 (2H, t, J 7 |
| | | | | | | | Hz); 6.35-6.4 |
| | | | | | | | (1H, m); 6.45 |
| | | | | | | | (1H, m); 6.8- |
| | | | | | | | 6.9 (1H, m) |
| 22 | A (chloro- | H | OCH₃ | 2-((4-iso- | H | 294 | 1.05 (6H, d, J |
| | alkyl phenol | | | propyl) | | | 7 Hz); 2.5-2.7 |
| | displacement) | | | piperazin-1- | | | (9H, m); 2.8 |
| | | | | yl)-ethoxy | | | (2H, t, J 7 |
| | | | | | | | Hz); 3.8 (3H, |
| | | | | | | | s); 4.1 (2H, t, |
| | | | | | | | J 7 Hz); 6.2 |
| | | | | | | | (1H, d, J 2 and |
| | | | | | | | 8 Hz); 6.3 (1H, |
| | | | | | | | d, J 2 Hz); |
| | | | | | | | 6.75 (1H, d, J |
| | | | | | | | 8 Hz) |
| 23 | A (chloro- | H | OCH₃ | 3-((4-iso- | H | 308 | 1.05 (6H, d, J |
| | alkyl phenol | | | propyl)- | | | 7 Hz); 1.9-2 |
| | displacement) | | | piperazin-1- | | | (2H, m); 2.5- |
| | | | | yl)propoxy | | | 2.7 (11H, m); |
| | | | | | | | 3.4 (2H, broad |
| | | | | | | | s); 3.8 (3H, s); |
| | | | | | | | 4 (2H, t, J 7 |
| | | | | | | | Hz); 6.2 (1H, |
| | | | | | | | d, J 2 and 8 |
| | | | | | | | Hz); 6.3 (1H, |
| | | | | | | | d, J 2 Hz); |
| | | | | | | | 6.75 (1H, d, J |
| | | | | | | | 8 Hz) |
| 24 | A (chloro- | CH₃ | H | 3-((4-CH₃) | H | 264 | 1.9-2 (2H, m); |
| | alkyl phenol | | | piperazin-1- | | | 2.1 (3H, s); |
| | displacement) | | | yl)propoxy | | | 2.35 (3H, s); |
| | | | | | | | 2.4-2.7 (10H, |
| | | | | | | | m); 4(2H, t, J |
| | | | | | | | 7 Hz); 6.25- |
| | | | | | | | 6.4 (2H, m); |
| | | | | | | | 6.9-7 (1H, m) |
| 25 | A (chloro- | CH₃ | H | 3-(piper- | H | 249 | 1.4-1.55 (2H, |
| | alkyl phenol | | | idino) | | | m); 1.6-1.7 |
| | displacement) | | | propoxy | | | (4H, m); 1.95- |
| | | | | | | | 2.05 (2H, m); |
| | | | | | | | 2.1 (3H, s); |
| | | | | | | | 2.4-2.6 (6H, |
| | | | | | | | m); 4(2H, t, J |
| | | | | | | | 7 Hz); 6.3-6.4 |
| | | | | | | | (2H, m); 6.9-7 |
| | | | | | | | (1H, m) |
| 26 | F (Mitsu- | H | OCH₃ | ((1-CH₃)- | H | 251 | See specific |
| | nobu) | | | piperidin-4- | | | example |
| | | | | yl)-methoxy | | | |
| 27 | F (Mitsu- | H | OCH₃ | 2-((1-CH₃) | H | 265 | 1.2-1.35 (2H, |
| | nobu) | | | piperidin-4- | | | m); 1.4-1.55 |
| | | | | yl)ethoxy | | | (1H, m); 1.65- |
| | | | | | | | 1.75 (4H, m), |
| | | | | | | | 1.85-1.95 (2H, |
| | | | | | | | m); 2.2 (3H, |
| | | | | | | | s); 2.8-2.9 |
| | | | | | | | (2H, m); 3.7 |
| | | | | | | | (3H, s); 3.9 |
| | | | | | | | (2H, t, J 7 Hz); |
| | | | | | | | 6.15 (1H, dd, J |
| | | | | | | | 2 and 8 Hz); |
| | | | | | | | 6.2 (1H, d, J 2 |
| | | | | | | | Hz); 6.65 (1H, |
| | | | | | | | d, J 8 Hz) |
| 28 | F (Mitsu- | H | H | 2-((1-CH₃) | H | 235 | 1.2-1.35 (2H, |
| | nobu) | | | piperidin-4- | | | m); 1.4-1.55 |
| | | | | yl)ethoxy | | | (1H, m); 1.6- |
| | | | | | | | 1.7 (4H, m); |
| | | | | | | | 1.85-1.95 (2H, |
| | | | | | | | m); 2.2 (3H, |
| | | | | | | | s); 2.8-2.9 |
| | | | | | | | (2H, m); 3.8- |
| | | | | | | | 3.9 (2H, m); |
| | | | | | | | 6.5-6.6 (2H, |
| | | | | | | | m); 6.7-6.8 |
| | | | | | | | (2H, m) |
| 29 | F (Mitsu- | H | H | (S)-(pyr- | H | 293 | (as N-Boc |
| | nobu) | | | rolidin-2- | | 193 | protected); |
| | | | | yl)methoxy | | | (dmso-d₆): |
| | | | | | | | 1.15 (9H, s), |
| | | | | | | | 1.4-1.7 (4H, |
| | | | | | | | m); 3-3.05 |
| | | | | | | | (2H, m); 3.4- |
| | | | | | | | 3.45 (1H, m); |
| | | | | | | | 3.5-3.55 (2H, |
| | | | | | | | m); 4.4 (2H, |
| | | | | | | | broad s); 6.2- |
| | | | | | | | 6.3 (2H, m); |
| | | | | | | | 6.4-6.5 (2H, m) |
| 30 | B (Halide | H | OCH₃ | 2-(iso- | H | | (as N-Boc |
| | displacement | | | propylam | | | protected) |
| | via alkoxy | | | ino)ethoxy | | | 1.15 (6H, d, J |
| | anion) | | | | | | 7 Hz); 1.45 |
| | | | | | | | (9H, s); 3.35- |
| | | | | | | | 3.5 (2H, m); |
| | | | | | | | 3.8 (3H, s); |
| | | | | | | | 3.9-4.1 (2H, |
| | | | | | | | m); 4.3-4.45 |
| | | | | | | | (1H, m); 6.2 |
| | | | | | | | (1H, dd, J 2 |
| | | | | | | | and 8 Hz); 6.3 |
| | | | | | | | (1H, d, J 2 |
| | | | | | | | Hz); 6.8 (1H, m) |
| 31 | B (Halide | H | Cl | 2-(iso- | H | | (as N-Boc |
| | displacement | | | propyl- | | | protected) |
| | via alkoxy | | | amino)- | | | 1.15 (6H, |
| | anion) | | | ethoxy | | | broad d, J 7 |
| | | | | | | | Hz); 1.45 (9H, |
| | | | | | | | s); 3.35-3.5 |
| | | | | | | | (2H, m); 3.9- |
| | | | | | | | 4.1 (2H, m); |
| | | | | | | | 4.3-4.45 (1H, |
| | | | | | | | m); 6.5 (1H, |
| | | | | | | | dd, J 2 and 8 |
| | | | | | | | Hz); 6.7 (1H, |
| | | | | | | | d, J2 Hz), 6.8 |
| | | | | | | | (1H, d, J 8 Hz) |
| 32 | C (Halide | (H?) | OCH₃ | (4-CH₃)- | H | 222 | 2.4 (3H, s); |
| | displacement | | | piperazin | | | 2.5-2.7 (4H, |
| | | | | -1-yl | | | m); 2.9-3.1 |
| | | | | | | | (4H, m); 3.8 |
| | | | | | | | (3H, s); 6.2- |
| | | | | | | | 6.4 (2H, m); |
| | | | | | | | 6.8-6.9 (1H, m) |
| 33 | C (Halide | H | OCH₃ | 4-(tert- | H | | (as N-Boc |
| | displacement | | | butoxy- | | | protected) |
| | | | | carbonyl) | | | 1.4 (9H, s); |
| | | | | piperazin | | | 2.8-2.9 (4H, |
| | | | | -1-yl | | | m); 3.5-3.6 |
| | | | | | | | 94H, m); 3.75 |
| | | | | | | | (3H, s); 6.1- |
| | | | | | | | 6.25 (2H, m); |
| | | | | | | | 6.65-6.8 (1H, m) |
| 34 | C (Halide | H | H | 4-(tert- | H | 278 | (as N-Boc |
| | displacement) | | | butoxy- | | | protected) |
| | | | | carbonyl) | | | 1.4 (9H, s); |
| | | | | piperazin | | | 2.85-2.95 (4H, |
| | | | | -1-yl | | | m); 3.4 (2H, |
| | | | | | | | broad s); 3.45- |
| | | | | | | | 3.55 (4H, m); |
| | | | | | | | 6.6 (2H, d, J 8 |
| | | | | | | | Hz); 6.75 (2H, |
| | | | | | | | d, J 8Hz) |
| 35 | C (Halide | H | H | 4-(iso- | H | 220 | 1.0 (6H, d, J 7 |
| | displacement) | | | propyl)- | | | Hz); 2.55-2.7 |
| | | | | piperazin | | | (6H, m); 2.95- |
| | | | | -1-yl | | | 3.05 (4H, m, |
| | | | | | | | 3.35 (2H, |
| | | | | | | | broad s); 6.5- |
| | | | | | | | 6.65 (2H, m); |
| | | | | | | | 6.7-6.8 (2H, m) |
| 36 | D (piperazine | H | H | 4-(carbamo- | H | 235 | 2.6-2.7 (4H, |
| | alkylation | | | ylmethyl) | | | m); 2.9-3.1 |
| | | | | piperazin | | | (4H, m); 3.4 |
| | | | | -1-yl | | | (2H, broad s); |
| | | | | | | | 5.4 (1H, broad |
| | | | | | | | s); 6.55 (2H, d, |
| | | | | | | | J 8Hz); 66.7 |
| | | | | | | | (2H, d, J 8Hz); |
| | | | | | | | 7 (1H, broad s) |
| 37 | D (piperazine | H | H | 4-(cyclo- | H | 274 | 0.7-0.9 (2H, |
| | alkylation | | | hexyl- | | | m); 1.1-1.3 |
| | | | | methyl)- | | | (3H, m); 1.4- |
| | | | | piperazin | | | 1.5 (1H, m); |
| | | | | -1-yl | | | 1.6-1.8 (5H, |
| | | | | | | | m); 2.1 (2H, d, |
| | | | | | | | J 7 Hz); 2.4- |
| | | | | | | | 2.55 (4H, m); |
| | | | | | | | 2.9-3 (4H, m); |
| | | | | | | | 3.35 (2H, |
| | | | | | | | broad s); 6.5- |
| | | | | | | | 6.65 (2H, m); |
| | | | | | | | 7.8-7.9 (2H, m) |
| 38 | D (piperazine | H | H | 4-(((3-Cl) | H | 302/ | See specific |
| | alkylation) | | | phenyl) | | 304 | example |
| | | | | methyl) | | | |
| | | | | piperazin | | | |
| | | | | -1-yl | | | |
| 39 | D (piperazine | H | H | 4-(((3- | H | 293 | 2.5-2.6 (4H, |
| | alkylation) | | | cyano)- | | | m); 2.9-3.1 |
| | | | | phenyl)- | | | (4H, m); 3.5 |
| | | | | methyl)- | | | (2H, s); 6.5- |
| | | | | piperazin | | | 6.6 (2H, m); |
| | | | | -1-yl | | | 6.7-6.8 (2H, |
| | | | | | | | m); 7.3-7.4 |
| | | | | | | | (1H, m); 7.5- |
| | | | | | | | 7.6 (2H, m); |
| | | | | | | | 7.6 (1H, m) |
| 40 | D (piperazine | H | H | 4-(((3- | H | | 2.5-2.6 (4H, |
| | alkylation) | | | OCH₃)- | | | m); 2.9-3.1 |
| | | | | phenyl)- | | | (4H, m); 3.45 |
| | | | | methyl)- | | | (2H, s), 3.75 |
| | | | | piperazin | | | (3H, s); 6.5- |
| | | | | -1-yl | | | 6.6 (2H, m); |
| | | | | | | | 6.7-6.8 (3H, |
| | | | | | | | m); 6.8-6.9 |
| | | | | | | | (2H, m); 7.2- |
| | | | | | | | 7.3 (1H, m) |
| 41 | C (Halide | H | Cl | (4-CH₃)- | H | | 2.35 (3H, s); |
| | displacement) | | | piperazin | | | 2.5-2.7 (4H, |
| | | | | -1-yl | | | m), 2.9-3 (4H, |
| | | | | | | | m); 3.5 (2H, |
| | | | | | | | broad s); 6.5 |
| | | | | | | | (1H, dd, J 2 |
| | | | | | | | and 8 Hz); 6.7 |
| | | | | | | | (1H, d, J 2 Hz); |
| | | | | | | | 6.9 (1H, d, J 8 |
| | | | | | | | Hz) |
| 42 | C (Halide | H | OCH₃ | 4-(iso- | H | | 1.1 (6H, d, J 7 |
| | displacement) | | | propyl)- | | | Hz); 2.6-2.7 |
| | | | | piperazin | | | (6H, m); 2.9- |
| | | | | -1-yl | | | 3.1 (4H, m); |
| | | | | | | | 3.75 (3H, s); |
| | | | | | | | 6.15-6.3 (2H, |
| | | | | | | | m); 6.7 (1H, d, |
| | | | | | | | J 8 Hz) |
| 43 | C (Halide | H | F | 4-(iso- | H | | 1.1 (6H, d, J 7 |
| | displacement) | | | propyl)- | | | Hz); 2.6-2.7 |
| | | | | piperazin | | | (6H, m); 2.9-3 |
| | | | | -1-yl | | | (4H, m); 3.5 |
| | | | | | | | (H, broad s); |
| | | | | | | | 6.3-6.4 (2H, |
| | | | | | | | m); 6.7-6.8 |
| | | | | | | | (1H, m) |
| 44 | C (Halide | H | (4-CH₃)- | H | H | 192 | 2.25 (3H, s); |
| | displacement) | | piperazin | | | | 2.45-2.5 (4H, |
| | | | -1-yl | | | | m); 3.1-3.2 |
| | | | | | | | (4H, m), 3.6 |
| | | | | | | | (2H, broad s); |
| | | | | | | | 6.1 (1H, dd, J |
| | | | | | | | 2 and 8 Hz); |
| | | | | | | | 6.2 (1H, m; 6.3 |
| | | | | | | | (1H, d, J 2 and |
| | | | | | | | 8 Hz); 6.95 |
| | | | | | | | (1H, t, J 8 Hz) |
| 45 | C (Halide | CH₃ | H | (4-CH₃)- | H | 206 | 2.1 (3H, s); 2.3 |
| | displacement) | | | piperazin | | | (3H, s); 2.5- |
| | | | | -1-yl | | | 2.6 (4H, m); |
| | | | | | | | 2.9-3.1 (4H, |
| | | | | | | | m); 6.5-6.6 |
| | | | | | | | (1H, m); 6.6- |
| | | | | | | | 6.7 (2H, m) |
| 46 | D (piperazine | H | H | (4-(2- | H | 249 | 2.2 (6H, s); |
| | alkylation) | | | dimethyl | | | 2.3-2.4 (2H, |
| | | | | amino- | | | m); 2.4-2.5 |
| | | | | ethyl))- | | | (2H, m), 2.5- |
| | | | | piperazin | | | 2.6 (4H, m); |
| | | | | -1-yl | | | 2.95-3.05 (4H, |
| | | | | | | | m); 6.55 (2H, |
| | | | | | | | d, J 8 Hz); |
| | | | | | | | 6.75 (2H, d, J |
| | | | | | | | 8 Hz) |
| 47 | D (piperazine | H | H | (4-((2- | H | | 2.5-2.6 (6H, |
| | alkylation) | | | methoxy) | | | m); 2.95-3.05 |
| | | | | ethyl))- | | | (4H, m); 3.3 |
| | | | | piperazin | | | (3H, s); 3.5 |
| | | | | -1-yl | | | (2H, t, J 7 Hz); |
| | | | | | | | 6.55 (2H, d, J |
| | | | | | | | 8 Hz); 6.75 |
| | | | | | | | (2H, d, J 8 Hz) |
| 48 | D (piperazine | H | H | (4-(3- | H | | See specific |
| | alkylation) | | | dimethyl | | | example |
| | | | | amino- | | | |
| | | | | propyl))pipe | | | |
| | | | | razin-1-yl | | | |
| 49 | E (amide | H | H | (N-(2- | H | 250 | dmso-d₆: 0.8- |
| | formation) | | | diethyl- | | | 1.0 (6H, m) |
| | | | | amino)- | | | 2.3-2.6 (6H, |
| | | | | ethyl)- | | | m); 3.0 (3H, |
| | | | | (N-methyl)) | | | broad s); 3.35- |
| | | | | carbamoyl | | | 3.5 (2H, m); |
| | | | | | | | 545 (2H, |
| | | | | | | | broad s); 6.5 |
| | | | | | | | (2H, d, J 8 |
| | | | | | | | Hz); 7.1 (2H, |
| | | | | | | | d, J 8 Hz) |
| 50 | E (amide | H | (N-(2- | H | H | 208 | See specific |
| | formation) | | dimethyl | | | | example |
| | | | amino)- | | | | |
| | | | ethyl)car | | | | |
| | | | bamoyl | | | | |
| 51 | E (amide | H | (N-(2- | H | H | 250 | dmso-d₆: 0.8— |
| | formation) | | diethy- | | | | 1.0 (6H, 2 |
| | | | lamino)- | | | | broad m); 2.2- |
| | | | ethyl)- | | | | 2.8 (4H, 2 |
| | | | (N-methyl)) | | | | broad m); 2.9- |
| | | | carbamoyl | | | | 3.0 (3H, 2 |
| | | | | | | | broad s); 3.2- |
| | | | | | | | 3.5 (2H, 2 |
| | | | | | | | broad m); 5.2 |
| | | | | | | | (2H, broad s); |
| | | | | | | | 6.4 (1H, d, J 8 |
| | | | | | | | Hz); 6.5 (1H, |
| | | | | | | | d, J 2 Hz), 6.6 |
| | | | | | | | (1H, dd, J 2 |
| | | | | | | | and 8 Hz); |
| | | | | | | | 7.05 (1H, m) |
| 52 | G (amide | H | H | 2-(diethyla- | H | | 1.0-1.1 (6H, |
| | reduction) | | | mino)ethyl | | | m); 2.5-2.75 |
| | | | | | | | (8H, m); 3.6 |
| | | | | | | | (2H, bs); 6.62 |
| | | | | | | | (2H, d, J 8.4); |
| | | | | | | | 6.98 (2H, d, J |
| | | | | | | | 8.4) |
| 53 | A | H | H | (2-(4 | H | 236 | 2.3 (3H, s); |
| | | | | methyl | | | 2.3-2.7 (8H, |
| | | | | piperazino) | | | m); 2.75 (2H, |
| | | | | ethoxy | | | t, J 7 Hz)); |
| | | | | | | | 3.95 (2H, t J 7 |
| | | | | | | | Hz); 6.5-6.6 |
| | | | | | | | (2H, m); 6.7- |
| | | | | | | | 6.8 (2H, m) |
| 54 | C | H | F | (4- | H | 210 | 2.25 (3H, s); |
| | | | | methyl | | | 2.45-2.55 (4H, |
| | | | | piperazino) | | | m); 3.85-2.95 |
| | | | | | | | (4H, m), 3.55 |
| | | | | | | | (2H, bs); 6.25- |
| | | | | | | | 6.35 (2H, m); |
| | | | | | | | 6.75-6.85 (1H, |
| | | | | | | | m) |
| 55 | A | H | F | 4-(2- | H | 239 | 1.35-1.45 (2H, |
| | | | | (piperidino) | | | m); 1.55-1.65 |
| | | | | ethoxy) | | | (4H, m); 2.45- |
| | | | | | | | 2.5 (4H, m); |
| | | | | | | | 2.7 (2H, t, J 7 |
| | | | | | | | Hz); 3.4-3.5 |
| | | | | | | | (2H, bs); 4.0 |
| | | | | | | | (2H, t, J 7 Hz); |
| | | | | | | | 6.35-6.45 (1H, |
| | | | | | | | m); 6.5-6.55 |
| | | | | | | | (1H, m); 6.7- |
| | | | | | | | 6.8 (1H, m) |
| 56 | C | H | OCHF₂ | (4- | H | 258 | 2.4-2.5 (4H, |
| | | | | methyl | | | m); 3.85-3.95 |
| | | | | piperazino) | | | (4H, m); 3.5 |
| | | | | | | | (2H, bs); 6.4- |
| | | | | | | | 6.45 (2H, m); |
| | | | | | | | 6.6 (1H, t, J 65 |
| | | | | | | | Hz); 6.75-6.85 |
| | | | | | | | (1H, m) |
| 57 | F Boc | H | OCH₃ | 2-((1- | H | 323 | (dmso-d₆) |
| | denyative | | | tert- | | 267 | 1.3-4.4 (9H, |
| | | | | butoxyca- | | 223 | m); 1.65-1.75 |
| | | | | rbonyl) | | | (1H, m); 1.8- |
| | | | | pyrrolidin-2- | | | 1.95 (3H, m); |
| | | | | yl)methoxy | | | 3.15-3.25 (1H, |
| | | | | | | | m); 3.65-3.75 |
| | | | | | | | (1H, m); 3.7 |
| | | | | | | | (3H, s); 3.8- |
| | | | | | | | 3.9 (2H, m); |
| | | | | | | | 4.7 (2H, bs); |
| | | | | | | | 6.0 (1H, dd, J |
| | | | | | | | 2 and 8 Hz); |
| | | | | | | | 6.2 (1H, d, J 2 |
| | | | | | | | Hz); 6.6 (1H, |
| | | | | | | | d, J 8 Hz) |
| 58 | C | H | Cl | (4- | H | | 1.2 (6H, d, J 7 |
| | | | | isopropyl | | | Hz); 2.8-2.9 |
| | | | | piperazino) | | | (4H, m); 2.9- |
| | | | | | | | 3.0 (1H, m); |
| | | | | | | | 3.0-3.2 (4H, |
| | | | | | | | m), 3.5 (2H, |
| | | | | | | | bs); 6.5 (1H, |
| | | | | | | | dd, J 2 and 8 |
| | | | | | | | Hz); 6.7 (1H, |
| | | | | | | | d, J 2 Hz); 6.85 |
| | | | | | | | (1H, d J 8 Hz) |
| 59 | E | H | (2- | H | H | 244 | 2.0 (6H, s); |
| | | | dimethyl | | | | 2.2-2.3 (2H, |
| | | | aminoethyl) | | | | m); 2.85-2.95 |
| | | | sulfamoyl) | | | | (2H, m); 6.65- |
| | | | oyl) | | | | 6.75 (1H, m); |
| | | | | | | | 6.95-7.05 (2H, |
| | | | | | | | m); 7.05-7.15 |
| | | | | | | | (1H, m) |
| 60 | A | H | Cl | 2-(4- | H | 298 | 1.3 (6H, d, J 7 |
| | | | | isopropyl | | | Hz); 2.85-3.05 |
| | | | | piperazino) | | | (10H, m); 3.15 |
| | | | | ethoxy | | | (1H, sectuplet, |
| | | | | | | | J 7 Hz); 4.1 |
| | | | | | | | 92H, t, J 7 |
| | | | | | | | Hz); 6.65 (1H, |
| | | | | | | | dd, J 2 and 8 |
| | | | | | | | Hz); 6.8 (1H, |
| | | | | | | | d, J 8 Hz); 6.85 |
| | | | | | | | (1H, d, J 8 Hz) |
| 61 | C | H | H | ((1,2- | H | 206 | (dmso-d₆) |
| | | | | dimethyl) | | | 1.0 (3H, d, 16 |
| | | | | piperazin | | | Hz); 2.05-2.15 |
| | | | | -4-yl | | | (1H, m); 2.2 |
| | | | | | | | (3H, s); 2.15- |
| | | | | | | | 2.25 (2H, m); |
| | | | | | | | 2.55-2.65 (1H, |
| | | | | | | | m); 2.7-2.8 |
| | | | | | | | (1H, m); 3.15- |
| | | | | | | | 3.25 (2H, m); |
| | | | | | | | 6.5 (2H, d, J 8 |
| | | | | | | | Hz); 6.7 (2H, |
| | | | | | | | d, J 8 Hz) |
| 62 | H | H | H | 1-methyl | H | 191 | See specific |
| | | | | piperid-4-yl | | | example |
| 63 | H | H | H | 1- | H | 219 | 1.1 (6H, d, J 7 |
| | | | | (isopropyl) | | | Hz); 1.7-1.8 |
| | | | | piperid-4-yl | | | (2H, m); 11.8- |
| | | | | | | | 1.9 (2H, m), |
| | | | | | | | 2.2-2.3 (2H, |
| | | | | | | | m); 2.35-2.45 |
| | | | | | | | (1H, m); 2.75- |
| | | | | | | | 2.85 (1H, m); |
| | | | | | | | 3.0-3.1 (2H, |
| | | | | | | | m); 3.6 (2H, |
| | | | | | | | bs); 6.7 (2H, d, |
| | | | | | | | J 8 Hz); 7.05 |
| | | | | | | | (2H, d, J 8 Hz) |
| 64 | H | H | H | 1-(3-(N,N- | H | 262 | 1.7-1.9 (6H, |
| | | | | dimethyl | | | m); 2.05-2.15 |
| | | | | amino | | | (2H, m); 2.3 |
| | | | | propyl) | | | (6H, s); 2.3- |
| | | | | piperid-4-yl | | | 2.4 (2H, m); |
| | | | | | | | 2.4-2.5 (3H, |
| | | | | | | | m); 3.05-3.15 |
| | | | | | | | (2H, m); 3.6 |
| | | | | | | | (2H, bs); 6.65 |
| | | | | | | | (2H, d, J 8 |
| | | | | | | | Hz); 7.05 (2H, |
| | | | | | | | d, J 8 Hz) |
| 65 | I | H | OCH₃ | (4-methyl- | H | 279 | See specific |
| | | | | piperazino) | | | example |
| | | | | methylcarbo- | | | |
| | | | | nylamino | | | |
| 66 | A | H | F | (3-(1- | H | 255 | 1.9-2.0 (2H, |
| | | | | morpholino) | | | m); 2.4-2.45 |
| | | | | propoxy | | | (4H, m); 2.5 |
| | | | | | | | (2H, t, J 7.5 |
| | | | | | | | Hz); 3.5 (2H, |
| | | | | | | | bs); 3.65-3.75 |
| | | | | | | | (4H, m); 4.0 |
| | | | | | | | (2H, t, J 7.5 |
| | | | | | | | Hz); 6.33-6.38 |
| | | | | | | | (1H, m); 6.45 |
| | | | | | | | (1H, dd, J 2.5 |
| | | | | | | | and 12.5); 6.8 |
| | | | | | | | (1H, t, J 9 Hz) |
| 67 | A | H | F | (3-(1- | H | 241 | (dmso-d₆) |
| | | | | morpholino) | | | 2.4-2.5 (4H, |
| | | | | propoxy | | | m); 2.6 (2H, t, |
| | | | | | | | J 5.6 Hz); |
| | | | | | | | 3.54-3.6 (4H, |
| | | | | | | | m); 3.96 (2H, |
| | | | | | | | t, J 5.6 Hz); |
| | | | | | | | 6.27-6.35 (1H, |
| | | | | | | | m); 6.39 (1H, |
| | | | | | | | dd, J 2.5 and |
| | | | | | | | 13 Hz); 6.84 |
| | | | | | | | (1H, dd J 8.5 |
| | | | | | | | and 13 Hz) |
| 68 | A | H | F | 2-(1- | H | 225 | (dmso-d₆) |
| | | | | pyrrolidino) | | | 1.6-1.7 (4H, |
| | | | | ethoxy | | | m); 2.72 (2H, |
| | | | | | | | t, J 5.8 Hz), |
| | | | | | | | 3.15-3.35 (2H, |
| | | | | | | | m); 3.45-3.55 |
| | | | | | | | (2H, m); 3.95 |
| | | | | | | | (2H, t, J 5.8 |
| | | | | | | | Hz); 6.25-6.3 |
| | | | | | | | (1H, m); 6.39 |
| | | | | | | | (1H, dd, J 2.5 |
| | | | | | | | and 13 Hz); |
| | | | | | | | 6.83 (1H, t, J |
| | | | | | | | 9.4 Hz) |
| 69 | J | H | F | (1-methyl- | H | 239 | (MeOH-d₄) |
| | | | | piperidin-3- | | | 1.05-4.15 (1H, |
| | | | | yl)methoxy | | | m); 1.6-1.7 |
| | | | | | | | (1H, m); 1.7- |
| | | | | | | | 1.8 (2H, m); |
| | | | | | | | 1.9-2.0 (1H, |
| | | | | | | | m); 2.0-2.15 |
| | | | | | | | (2H, m); 2.33 |
| | | | | | | | (3H, s); 2.85- |
| | | | | | | | 2.95 (1H, m); |
| | | | | | | | 3.05-3.15 (1H, |
| | | | | | | | m); 3.75 (1H, |
| | | | | | | | dd, J 7.5 and |
| | | | | | | | 9.5); 3.84 (1H, |
| | | | | | | | dd, J 5.5 and |
| | | | | | | | 9.5); 6.40-6.45 |
| | | | | | | | (1H, m); 6.51 |
| | | | | | | | (1H, dd, J 2.5 |
| | | | | | | | and 13 Hz); |
| | | | | | | | 6.84 (1H, t, J |
| | | | | | | | 9.5 Hz) |
| 70 | A | H | F | 3-(1- | H | 239 | 1.7-1.8 (4H, |
| | | | | pyrrolidino) | | | m); 1.95-2.05 |
| | | | | propoxy | | | (2H, m); 2.45- |
| | | | | | | | 2.55 (4H, m); |
| | | | | | | | 2.6 (2H, t, J 7 |
| | | | | | | | Hz); 3.5 (2H, |
| | | | | | | | bs); 4.0 (2H, t, |
| | | | | | | | J 7 Hz); 6.33- |
| | | | | | | | 6.37 (1H, m); |
| | | | | | | | 6.45 (1H, dd, J |
| | | | | | | | 2.5 and 13 |
| | | | | | | | Hz); 6.80 (1H, |
| | | | | | | | t, J 9 Hz) |
| 71 | J | H | F | (octahydro- | H | 279 | (dmso-d₆) |
| | | | | 2H- | | | 1.1-1.8 (9H, |
| | | | | quinolizin-1- | | | m); 1.9-2.0 |
| | | | | yl)methoxy | | | (3H, m); 2.7- |
| | | | | | | | 2.8 (2H, m); |
| | | | | | | | 3.9-4.0 (1H, |
| | | | | | | | m); 4.05-4.15 |
| | | | | | | | (1H, m); 4.9 |
| | | | | | | | (1H, bs); 6.35- |
| | | | | | | | 6.4 (1H, m); |
| | | | | | | | 6.45-6.5 (1H, |
| | | | | | | | m); 6.85-6.95 |
| | | | | | | | (1H, m) |
| 72 | J Boc | H | F | (1-(tert- | H | 225, | (dmso-d₆) |
| | derivative | | | butoxy- | | 325 | 1.5-1.6 (1H, |
| | | | | carbonyl) | | | m); 1.65-1.8 |
| | | | | piperidin-3- | | | (2H, m); 2.75- |
| | | | | yl)methoxy | | | 2.85 (2H, m); |
| | | | | | | | 3.65-3.8 (4H, |
| | | | | | | | m); 3.8-4.05 |
| | | | | | | | (2H, m); 5.0 |
| | | | | | | | (2H, bs); 6.25- |
| | | | | | | | 6.30 (1H, m); |
| | | | | | | | 6.39 (1H, dd, J |
| | | | | | | | 2.5 and 13 |
| | | | | | | | Hz), 6.83 (1H, |
| | | | | | | | dd, J 9.1 and |
| | | | | | | | 9.8 Hz) |
| 73 | A | H | F | 2-(1-tert- | H | 340 | (dmso-d₆) |
| | | | | butoxy | | | 1.45 (9H, s); |
| | | | | carbonyl) | | | 2.4-2.5 (4H, |
| | | | | piperazin-4- | | | m); 2.65 (2H, |
| | | | | yl)ethoxy | | | t, J 6.5 Hz); |
| | | | | | | | 3.25-3.35 (4H, |
| | | | | | | | m), 4.0 (2H, t, |
| | | | | | | | J 6.5 Hz); 4.95 |
| | | | | | | | (2H, bs); 6.25- |
| | | | | | | | 6.35 (1H, m); |
| | | | | | | | 6.4 (1H, dd, J |
| | | | | | | | 2.5 and 13 |
| | | | | | | | Hz); 6.85 (1H, |
| | | | | | | | t, J 9 Hz) |
| 74 | D | H | H | (4-(2-tert- | H | | See specific |
| | | | | butoxy- | | | example |
| | | | | carbonylam- | | | |
| | | | | ino)ethyl) | | | |
| | | | | piperazin | | | |
| | | | | -1-yl | | | |
| 75 | D | H | H | (4-(2-N- | H | | See specific |
| | | | | tert- | | | example |
| | | | | butoxy- | | | |
| | | | | carbonyl-N- | | | |
| | | | | ethylamino) | | | |
| | | | | ethyl) | | | |
| | | | | piperazin | | | |
| | | | | -1-yl | | | |
| 76 | F | H | F | (N-tert- | H | 311 | 1.45 (9H, s); |
| | | | | butoxy- | | | 1.8-1.9 (1H, |
| | | | | carbonyl- | | | m), 1.9-2.1 |
| | | | | pyrrolidin-2- | | | (2H, m); 2.1- |
| | | | | (R)-yl) | | | 2.2 (1H, m); |
| | | | | methoxy | | | 3.25-3.45 (2H, |
| | | | | | | | m); 3.75-4 |
| | | | | | | | (1H, m); 4.0- |
| | | | | | | | 4.2 (2H, m); |
| | | | | | | | 6.3-6.4 (1H, |
| | | | | | | | m); 6.4-6.5 |
| | | | | | | | (1H, m), 6.8- |
| | | | | | | | 6.95 (1H, m) |
| 77 | F | H | F | (N-tert- | H | 311 | 1.45 (9H, s); |
| | | | | butoxy- | | | 1.8-4.9 (1H, |
| | | | | carbonyl- | | | m); 1.9-2.1 |
| | | | | pyrrolidin-2- | | | (2H, m); 2.1- |
| | | | | (S)-yl) | | | 2.2 (1H, m); |
| | | | | methoxy | | | 3.25-3.45 (2H, |
| | | | | | | | m); 3.55 (2H, |
| | | | | | | | bs); 3.75-4 |
| | | | | | | | (1H, m); 4.0- |
| | | | | | | | 4.2 (2H, m); |
| | | | | | | | 6.3-6.4 (1H, |
| | | | | | | | m); 6.4-6.5 |
| | | | | | | | (1H, m); 6.8- |
| | | | | | | | 6.95 (1H, m) |
| 78 | A | H | F | 4-(1- | H | | 1.7-1.85 (2H, |
| | | | | imidazolyl) | | | m); 1.95-2.1 |
| | | | | buthoxy | | | (2H, m); 3.95 |
| | | | | | | | (2H, t, J 7 Hz), |
| | | | | | | | 4.05 (2H, t, J 7 |
| | | | | | | | Hz); 6.45 (1H, |
| | | | | | | | m); 6.55 (1H, |
| | | | | | | | dd, J 2.5 and |
| | | | | | | | 13 Hz); 6.75 |
| | | | | | | | (1H, t, J 9.5 |
| | | | | | | | Hz); 6.9 (1H, |
| | | | | | | | s); 7.1 (1H, s); |
| | | | | | | | 7.5 (1H, s) |
| 79 | C | H | F | (1,2- | H | 224 | See specific |
| | | | | dimethyl) | | | synthesis |
| | | | | piperazin | | | |
| | | | | -4-yl | | | |
| 80 | C | H | F | (1,2,6- | H | 238 | 1.15 (6H, d, II |
| | | | | rimethyl) | | | 7 Hz); 2.3 (3H, |
| | | | | piperazin | | | s); 2.4-2.5 |
| | | | | -4-yl | | | (2H, m); 2.5- |
| | | | | | | | 2.6 (2H, m); |
| | | | | | | | 3.1-3.2 (2H, |
| | | | | | | | m); 3.55 (2H, |
| | | | | | | | bs); 6.3-6.5 |
| | | | | | | | (2H, m), 6.8 |
| | | | | | | | (1H, t, J 9.5 Hz) |
| 81 | G | H | H | 2-(4- | H | | See specific |
| | | | | methyl- | | | synthesis |
| | | | | piperazino) | | | |
| | | | | ethyl | | | |
| 82 | G | H | H | (4- | H | | See specific |
| | | | | methyl- | | | synthesis |
| | | | | piperazino) | | | |
| | | | | carbonyl- | | | |
| | | | | methyl | | | |
| 83 | G | H | H | 2-(1,2- | H | 234 | 1.15 (3H, d, |
| | | | | dimethyl- | | | J 7.5 Hz); 1.85- |
| | | | | piperazi-4- | | | 1.95 (1H, m), |
| | | | | yl)ethyl | | | 2.1-2.25 (1H, |
| | | | | | | | m); 2.25-2.4 |
| | | | | | | | (1H, m); 2.3- |
| | | | | | | | 2.4 (1H, m) |
| | | | | | | | 2.5-2.6 (2H, |
| | | | | | | | m); 2.65-2.75 |
| | | | | | | | (2H, m); 2.8- |
| | | | | | | | 2.9 (2H, m); |
| | | | | | | | 2.9-3.0 (2H, |
| | | | | | | | m); 3.6 (2H, |
| | | | | | | | bs); 6.65 (2H, |
| | | | | | | | d, J 8.5); 7.0 |
| | | | | | | | (2H, d, J 8.5) |
| 84 | G | H | H | (1,2- | H | 248 | 0.95 and 1.05 |
| | | | | dimethyl- | | | (3H, two d, J 7 |
| | | | | piperazin-4- | | | Hz); 1.9-2.1 |
| | | | | yl)carbon- | | | (1H, m), 2.1- |
| | | | | ylmethyl | | | 2.2 (1H, m); |
| | | | | | | | 2.2 (3H, s); |
| | | | | | | | 2.4-2.6 (1H, |
| | | | | | | | m); 2.7-3.0 |
| | | | | | | | (1H, m); 3.1- |
| | | | | | | | 3.3 (1H, m); |
| | | | | | | | 3.6 (2H, s); |
| | | | | | | | 3.6-3.7 (1H, |
| | | | | | | | m), 4.3-4.45 |
| | | | | | | | (1H, m); 6.6 |
| | | | | | | | (2H, d, J 8.5 |
| | | | | | | | Hz); 7.0 (2H, |
| | | | | | | | d, J 8.5 Hz) |
| 85 | A | H | F | (4-(2- | H | | 1.9-2.0 (2H, |
| | | | | methoxy- | | | m); 2.4-2.7 |
| | | | | ethyl) | | | (12H, m); 3.4 |
| | | | | piperazino) | | | (3H, s); 3.5- |
| | | | | propoxy | | | 3.6 (4H, m); |
| | | | | | | | 3.95-4.1 (2H, |
| | | | | | | | m); 6.3-6.4 |
| | | | | | | | (1H, m); 6.45 |
| | | | | | | | (1H, dd, J 2.5 |
| | | | | | | | and 13 Hz); |
| | | | | | | | 6.8 (1H, t, J |
| | | | | | | | 9.5 Hz) |
| 86 | A | H | F | (4-(2-N,N- | H | | 1.9-2.0 (2H, |
| | | | | dimethyl | | | m); 2.3 (6H, |
| | | | | aminoethyl) | | | s); 2.4-2.7 |
| | | | | piperazino) | | | (14H, m), 3.6 |
| | | | | propoxy | | | (2H, bs); 3.95- |
| | | | | | | | 4.1 (2H, m); |
| | | | | | | | 6.3-6.4 (1H, |
| | | | | | | | m); 6.45 (1H, |
| | | | | | | | dd, J 2.5 and |
| | | | | | | | 13 Hz); 6.8 |
| | | | | | | | (1H, t, J 9.5 Hz) |

Method K

General method for the synthesis of 4-aminoanilines of type K.

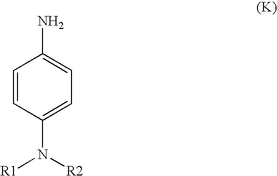

Step 1: A solution of 4-fluoronitrobenzene (1.41 g, 1.06 mL, 0.01 mol), N,N-diisopropylethylamine (1.1 equiv), and amine (1.1 equiv) in N,N-dimethylformamide (8-10 mL) was heated at 100° C. for 48 h in a sealed tube. The reaction mixture was cooled to room temperature and concentrated. The residue was purified via column chromatography on silica gel (gradient elution with 0 to 10% methanol-dichloromethane) to afford the nitroaniline.

Step 2: 10% Palladium on carbon (0.05 g) was added to a solution of the nitroaniline (0.001 mol) in ethanol (50 mL) under a H₂(g) atmosphere (via balloon). The reaction mixture stirred at r.t. overnight and was then filtered through celite. The filtrate was concentrated to afford a dark yellow oil.

Method L

General method for the synthesis of 2,5-diaminopyridines of type L.

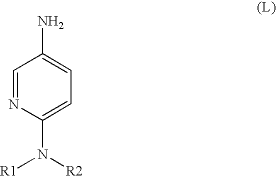

Step 1: A solution of 2-chloro-5-nitropyridine (0.317 g, 1.06 mL, 0.002 mol), N,N-diisopropylethylamine (1.1 equiv), and amine (1.1 equiv) in acetonitrile (40 mL) was refluxed for 24 h. The reaction mixture was cooled to room temperature and concentrated. The brown residue was used without purification.

Step 2: The diaminopyridine was prepared from the aminonitropyridine using the procedure in step 2 of method K.

Method M

General method for the synthesis of 4-aminoalkoxyanilines of type M.

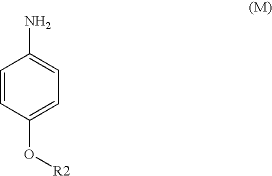

Step 1: A solution of 4-fluoronitrobenzene (0.141 g, 0.106 mL, 0.001 mol), aminoalcohol (1.1 equiv) in tetrahydrofuran (8-10 mL) was cooled to 0° C. in a sealed tube. A solution of KHMDS (0.5 M in toluene) was added dropwise, and the reaction mixture was allowed to reach room temperature. The mixture was partitioned between sat. aq. K₂CO₃ and ethylacetate. The organic layer was separated, dried over anhydrous Na₂SO₄, and concentrated. The residue was purified via column chromatography on silica gel (gradient elution with 0 to 10% methanol-dichloromethane) to afford the alkoxynitrobenzene.

Step 2: The alkoxyaniline was prepared from the alkoxynitrobenzene using the procedure in step 2 of method K.

Method N

General method for the synthesis of 4-[P-aminoalcohol]-alkoxyanilines of type N

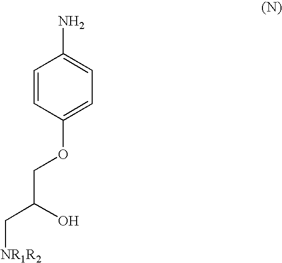

Step 1: A solution of 1,2-epoxy-3-(4-nitrophenoxy)propane (1.95 g, 0.01 mol), N,N-diisopropylethylamine (1.1 equiv), and amine (1.1 equiv) in methanol (60 mL) was refluxed for 24 h. The reaction mixture was cooled to room temperature and concentrated. The residue was purified via column chromatography on silica gel (gradient elution with 0 to 20% methanol-dichloromethane) to afford the aminoalkoxynitrobenzene.

Step 2: The aminoalkoxyaniline was prepared from the alkoxynitrobenzene using the procedure in step 2 of method K.

Table of anilines made.

| Aniline | MW | (MH+) | Method |
|---|---|---|---|
| 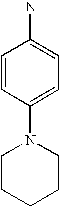 | 176.26 | 177 | K |
| 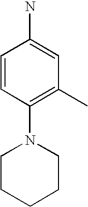 | 190.29 | 191 | K |
| 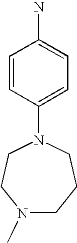 | 205.31 | 206 | K |
| 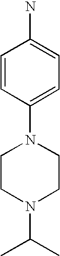 | 219.33 | 220 | K |
| 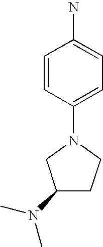 | 205.31 | 206 | K |
| 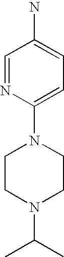 | 220.32 | 221 | L |
| 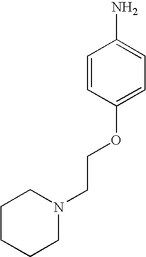 | 220.32 | 221 | M |
| 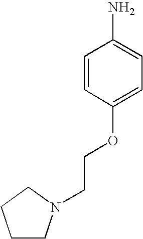 | 206.29 | 207 | M |
| 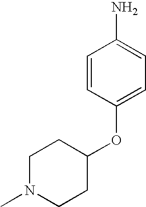 | 206.29 | 207 | M |
| 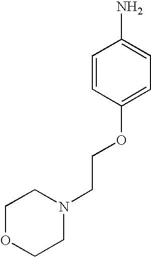 | 222.29 | 223 | M |
| 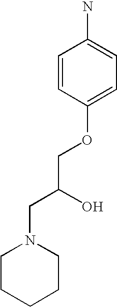 | 250.34 | 251 | N |

### 4-(3-Piperidin-1-yl-propoxy)-phenylamine

4-(3-piperidin-1-yl-propoxy)-phenylamine was prepared according to the method described in WO 03/018021.

Analytical Methods:

Unless otherwise indicated all HPLC analyses were run on an HP-1000 or HP-1050 system with an HP Zorbax SB-C₁₈ (5μ) reverse phase column (4.6×150 mm) run at 30° C. with a flow rate of 1.00 mL/min. The mobile phase used solvent A (H₂O/0.1% TFA) and solvent B (CH₃CN/0.1% TFA) with a 20 min gradient from 10% to 90% CH₃CN. The gradient was followed by a 2 min return to 10% CH₃CN and a 3 min flush. The peaks of interest eluted on the LC profiles at the times indicated.

LC-MS Methods:

Method A:

Samples were run on an HP-1100 system with an HP Zorbax SB-C₈ (5μ) reverse phase column (4.6×50 mm) run at 30° C. with a flow rate of 0.75 mL/min.

The mobile phase used solvent A (H₂O/0.1% ACOH) and solvent B (CH₃CN/0.1% ACOH) with a 10 min gradient from 10% to 90% CH₃CN. The gradient was followed by a 1 min return to 10% CH₃CN and a 2 min flush.

The peaks of interest eluted on the LC profiles at the times indicated.

Method B:

Samples were run on an HP-1100 system with an HP Zorbax SB-C₈ (5μ) reverse phase column (4.6×50 mm) run at 30° C. with a flow rate of 1.5 mL/min.

The mobile phase used solvent A (H₂O/0.1% AcOH) and solvent B (CH₃CN/0.1% AcOH) with a 5 min gradient from 10% to 90% CH₃CN. The gradient was followed by a 0.5 min return to 10% CH₃CN and a 1.5 min flush.

Proton NMR Spectra:

Unless otherwise indicated all ¹H NMR spectra were run on an Varian series Mercury 300 or 400 MHz instrument. All observed protons are reported as parts per million (ppm) downfield from tetramethylsilane (TMS) or other internal reference in the appropriate solvent indicated.

### EXAMPLE 1

### 4-(2-((2,6-Dimethylphenyl)oxy)-1H-benzimidazol-1-yl)-N-(4-(4-methyl-1-piperazinyl)phenyl)-2-pyrimidinamine

### Step A: 2-Chloro-benzoimidazole-1-carboxylic acid tert-butyl ester

Di-tert-butyldicarbonate (12.270 g, 56.221 mmol) was added to a solution of 2-chlorobenzimidazole (8.41 g, 55.119 mmol) and triethylamine (20 mL) in isopropanol (90 mL). The reaction mixture was stirred at room temperature for 19 h and then concentrated. The resulting material was purified via column chromatography on silica gel (eluting with dichloromethane) to afford 2-chloro-benzoimidazole-1-carboxylic acid tert-butyl ester as a white solid.

### Step B: 2-(2,6-Dimethyl-phenoxy)-1H-benzoimidazole

Cesium carbonate (2.643 g, 8.112 mmol) was added to a solution of 2-chloro-benzoimidazole-1-carboxylic acid tert-butyl ester (1.000 g, 3.957 mmol) and 2,6-dimethylphenol (3.867 g, 31.66 mmol) in isopropanol (10 mL). The mixture was heated at 150° C. for 22 h and then cooled to room temperature. The reaction mixture was partitioned between ethyl acetate and 2.0 N sodium hydroxide solution. The aqueous phase was separated and extracted with ethyl acetate and dichloromethane. The combined organic phases were washed with brine, dried over anhydrous magnesium sulfate, filtered, and concentrated to afford an orange-brown solid. Trituration with dichloromethane and filtering afforded 2-(2,6-dimethyl-phenoxy)-1H-benzoimidazole as an off-white solid. MS (MH⁺) 239.2; Calculated 238.11 for C₁₅H₁₄N₂O.

### Step C: 1-(2-Chloro-pyrimidin-4-yl)-2-(2,6-dimethyl-phenoxy)-1H-benzoimidazole

Sodium hydride (60% dispersion in mineral oil, 0.084 g, 2.10 mmol) was added to a solution of 2-(2,6-dimethyl-phenoxy)-1H-benzoimidazole (0.500 g, 2.10 mmol) in N,N-dimethylformamide (20 mL). 2,4-Dichloropyrimidine (0.298 g, 2.00 mmol) was added and the mixture stirred at room temperature for 18 h. Saturated ammonium chloride solution was added and the mixture was partitioned between dichloromethane and water. The aqueous phase was separated and extracted with dichloromethane. The combined organic phases were washed with brine, dried over anhydrous magnesium sulfate, filtered, and concentrated to afford a brown solid. This solid was purified via column chromatography on silica gel (eluting with 0-50% ethyl acetate-hexane) to afford 1-(2-chloro-pyrimidin-4-yl)-2-(2,6-dimethyl-phenoxy)-1H-benzoimidazole as an off-white solid. MS (MH⁺) 351.1; Calculated 350.09 for C₁₉H₁₅ClN₄O.

### Step D: 4-(2-((2,6-Dimethylphenyl)oxy)-1H-benzimidazol-1-yl)-N-(4-(4-methyl-1-piperazinyl)phenyl)-2-pyrimidinamine

4-(4-Methylpiperazino)aniline (0.160 g, 0.834 mmol) was added to a solution of 1-(2-chloro-pyrimidin-4-yl)-2-(2,6-dimethyl-phenoxy)-1H-benzoimidazole (0.266 g, 0.758 mmol) in acetic acid (10 mL). The mixture stirred at 110° C. for 16 h and was then concentrated. The residue was partitioned between dichloromethane and saturated sodium bicarbonate solution. The aqueous phase was separated and extracted with dichloromethane. The combined organic phases were washed with brine, dried over anhydrous magnesium sulfate, filtered and concentrated to afford a brown solid. This material was purified via column chromatography on silica gel (gradient elution with 0-100% dichloromethane-(90: 10:1, dichloromethane/methanol/ammonium hydroxide)) to afford a yellow solid. Trituration with hexane afforded 4-(2-((2,6-dimethylphenyl)oxy)-1H-benzimidazol-1-yl)-N-(4-(4-methyl-1-piperazinyl)phenyl)-2-pyrimidinamine as a pale yellow solid. MS (MH⁺) 506.4; Calculated 505.26 for C₃₀H₃₁N₇O.

### EXAMPLE 2

### 4-(2-((2-(Methyloxy)phenyl)oxy)-1H-benzimidazol-1-yl)-N-(4-(4-morpholinyl)phenyl)-2-pyrimidinamine

### Step A: 2-Chloro-benzoimidazole-1-carboxylic acid tert-butyl ester

Prepared as detailed above

### Step B: 2-(2-Methoxy-phenoxy)-1H-benzoimidazole

Cesium carbonate (3.965 g, 12.17 mmol) was added to a solution of 2-chloro-benzoimidazole-1-carboxylic acid tert-butyl ester (1.500 g, 5.936 mmol) and 2-methoxyphenol (5.90 g, 5.20 mL, 47.5 mmol) in isopropanol (25 mL). The mixture was heated at 150° C. for 17 h and was then cooled to room temperature. The reaction mixture was partitioned between ethyl acetate and 2.0 N sodium hydroxide solution. The aqueous phase was separated and extracted with ethyl acetate and dichloromethane. The combined organic phases were washed with brine, dried over anhydrous magnesium sulfate, filtered, and concentrated to afford an off-white solid. Trituration with ethyl acetate and filtering afforded 2-(2-methoxy-phenoxy)-1H-benzoimidazole as a white solid. MS (MH⁺) 240.8; Calculated 240.09 for C₁₄H₁₂N₂O₂.

### Step C: 1-(2-Chloro-pyrimidin-4-yl)-2-(2-methoxy-phenoxy)-1H-benzoimidazole

Sodium hydride (60% dispersion in mineral oil, 0.116 g, 2.91 mmol) was added to a solution of 2-(2-methoxy-phenoxy)-1H-benzoimidazole (0.700 g, 2.91 mmol) in N,N-dimethylformamide (30 mL). 2,4-Dichloropyrimidine (0.413 g, 2.77 mmol) was added and the mixture stirred at room temperature for 20 h. Saturated ammonium chloride solution was added and the mixture was partitioned between dichloromethane and water. The aqueous phase was separated and extracted with dichloromethane. The combined organic phases were washed with brine, dried over anhydrous magnesium sulfate, filtered, and concentrated to afford a brown solid. This solid was purified via column chromatography on silica gel (eluting with 0-50% ethyl acetate-hexane) to afford 1-(2-chloro-pyrimidin-4-yl)-2-(2-methoxy-phenoxy)-1H-benzoimidazole as a white solid. MS (MH⁺) 353.1; Calculated 352.07 for C₁₈H₁₃ClN₄O₂.

### Step D: 4-(2-((2-(Methyloxy)phenyl)oxy)-1H-benzimidazol-1-yl)-N-(4-(4-morpholinyl)phenyl)-2-pyrimidinamine

A resealable tube was charged with the 1-(2-chloro-pyrimidin-4-yl)-2-(2-methoxy-phenoxy)-1H-benzoimidazole (0.050 g, 0.142 mmol), 4-morpholinoaniline (0.035 g, 0.200 mmol), tris(dibenzylideneacetone)dipalladium (0.005 g, 0.006 mmol), 4,5-bis-diphenylphosphanyl-9,9-dimethyl-9H-xanthene (0.010 g, 0.017 mmol), and sodium carbonate (0.021 g, 0.199 mmol). The system was flushed with argon and toluene (1 mL) was added, followed by the addition of water (0.0030 mL, 0.14 mmol) (with stirring). The tube was flushed with argon and sealed. The mixture was heated at 100° C. for 18 h and then cooled to room temperature. The reaction mixture was filtered, and the filtrate was concentrated to afford a brown oil. The oil was purified via column chromatography (gradient elution with 0-50% ethyl acetate-hexane) to afford 4-(2-((2-(methyloxy)phenyl)oxy)-1H-benzimidazol-1-yl)-N-(4-(4-morpholinyl)phenyl)-2-pyrimidinamine as an off-white solid. MS (MH⁺) 495.2; Calculated 494.21 for C₂₈H₂₆N₆O₃.

### EXAMPLES 3 To 5

The following compounds were prepared using the procedure outlined above for the synthesis of 4-(2-((2-(methyloxy)phenyl)oxy)-1H-benzimidazol-1-yl)-N-(4-(4-morpholinyl)phenyl)-2-pyrimidinamine.

### EXAMPLE 3

### 4-(2-((2-(Methyloxy)phenyl)oxy)-1H-benzimidazol-1-yl)-N-(4-(4-methyl-1-piperazinyl)phenyl)-2-pyrimidinamine

MS (MH⁺) 508.2; Calculated 507.24 for C₂₉H₂₉N₇O₂.

### EXAMPLE 4

### 4-(4-(4-(2-((2-(Methyloxy)phenyl)oxy)-1H-benzimidazol-1-yl)-2-pyrimidinyl)-1-piperazinyl)phenylamine

MS (MH⁺) 494.2; Calculated 493.22 for C₂₈H₂₇N₇O₂.

### EXAMPLE 5

### N-(4-((2-(Dimethylamino)ethyl)oxy)phenyl)-4-(2-((2-(methyloxy)phenyl)oxy)-1H-benzimidazol-1-yl)-2-pyrimidinamine

MS (MH⁺) 497.3; Calculated 496.22 for C₂₈H₂₈N₆O₃.

### EXAMPLE 6

### N-(4-((2-((1-methylethyl)amino)ethyl)oxy)-3-(methyloxy)phenyl)-4-(2-((2-(methyloxy)phenyl)oxy)-1H-benzimidazol-1-yl)-2-pyrimidinamine

### Steps A to D: Isopropyl-[2-(2-methoxy-4-{4-[2-(2-methoxy-phenoxy)-benzoimidazol-1-yl]-pyrimidin-2-ylamino}-phenoxy)-ethyl]-carbamic acid tert-butyl ester

Isopropyl-[2-(2-methoxy-4-{4-[2-(2-methoxy-phenoxy)-benzoimidazol-1-yl]-pyrimidin-2-ylamino}-phenoxy)-ethyl]-carbamic acid tert-butyl ester was prepared as a pale yellow solid according to the procedures outlined for the preparation of 4-(2-((2-(methyloxy)phenyl)oxy)-1H-benzimidazol-1-yl)-N-(4-(4-morpholinyl)phenyl)-2-pyrimidinamine. MS (MH⁺) 641.1; Calculated 640.30 for C₃₅H₄₀N₆O₆.

### Step E: N-(4-((2-((1-methylethyl)amino)ethyl)oxy)-3-(methyloxy)phenyl)-4-(2-((2-(methyloxy)phenyl)oxy)-1H-benzimidazol-1-yl)-2-pyrimidinamine

A solution of isopropyl-[2-(2-methoxy-4-{4-[2-(2-methoxy-phenoxy)-benzoimidazol-1-yl]-pyrimidin-2-ylamino}-phenoxy)-ethyl]-carbamic acid tert-butyl ester (0.150 g, 0.234 mmol) in dichloromethane (2 mL) was cooled to 0° C. and trifluoroacetic acid (1 mL) was added dropwise. The resulting orange solution stirred at 0° C. for 2 h and was concentrated. The residue was partitioned between ethyl acetate and saturated sodium bicarbonate solution. The organic phase was separated and washed with brine, dried over anhydrous magnesium sulfate, filtered and concentrated to afford N-(4-((2-((1-methylethyl)amino)ethyl)oxy)-3-(methyloxy)phenyl)-4-(2-((2-(methyloxy)phenyl)oxy)-1H-benzimidazol-1-yl)-2-pyrimidinamine as an off-white solid. MS (MH⁺) 541.2; Calculated 540.25 for C₃₀H₃₂N₆O₄.

### EXAMPLES 7 TO 12

The following compounds were prepared using the procedure outlined above for the preparation of N-(4-((2-((1-methylethyl)amino)ethyl)oxy)-3-(methyloxy)phenyl)-4-(2-((2-(methyloxy)phenyl)oxy)-1H-benzimidazol-1-yl)-2-pyrimidinamine.

### EXAMPLE 7

### 4-(2-((2-(methyloxy)phenyl)oxy)-1H-benzimidazol-1-yl)-N-(3-(methyloxy)-4-(((2S)-2-pyrrolidinylmethyl)oxy)phenyl)-2-pyrimidinamine

MS (MH⁺) 539.2; Calculated 538.23 for C₃₀H₃₀N₆O₄.

### EXAMPLE 8

### 4-(2-((2-(methyloxy)phenyl)oxy)-1H-benzimidazol-1-yl)-N-(3-(methyloxy)-4-(((2R)-2-pyrrolidinylmethyl)oxy)phenyl)-2-pyrimidinamine

MS (MH⁺) 539.2; Calculated 538.23 for C₃₀H₃₀N₆O₄

### EXAMPLE 9

### N-(3-chloro-4-((2-((1-methylethyl)amino)ethyl)oxy)phenyl)-4-(2-((2-(methyloxy)phenyl)oxy)-1H-benzimidazol-1-yl)-2-pyrimidinamine

MS (MH⁺) 545.4; Calculated 544.20 for C₂₉H₂₉ClN₆O₃.

### EXAMPLE 10

### N-(4-((2-((1-methylethyl)amino)ethyl)oxy)phenyl)-4-(2-((2-(methyloxy)phenyl)oxy)-1H-benzimidazol-1-yl)-2-pyrimidinamine

MS (MH⁺) 511.3; Calculated 510.24 for C₂₉H₃₀N₆O₃.

### EXAMPLE 11

### 4-(2-((2,3-bis(methyloxy)phenyl)oxy)-1H-benzimidazol-1-yl)-N-(3-chloro-4-((2-((1-methylethyl)amino)ethyl)oxy)phenyl)-2-pyrimidinamine

MS (MH⁺) 575.2; Calculated 574.21 for C₃₀H₃₁ClN₆O₄.

### EXAMPLE 12

### 4-(2-((2,3-bis(methyloxy)phenyl)oxy)-1H-benzimidazol-1-yl)-N-(4-((2-((1-methylethyl)amino)ethyl)oxy)phenyl)-2-pyrimidinamine

MS (MH⁺) 541.2; Calculated 540.25 for C₃₀H₃₂N₆O₄.

### EXAMPLE 13

4-(2-(pyridine-2-ylmethoxy)-1H-benzo[d]imidazol-1-yl)-N-3,4,5-trimethoxyphenyl)-1,3,5-triazin-2-amine was prepared in a manner similar to that described in Examples 1, 2 and 6. MS (MH⁺) 486; Calculated 485.5 for C₂₅H₂₃N₇O₄.

Biological Assays

The following assays can be employed to determine the degree of activity of a compound as a protein kinase inhibitor. Compounds described herein have been tested in one or more of these assays, and have shown activity. Representative compounds of the invention (Examples 1-12) were tested and found to exhibit IC₅₀ values of at least <10 μM in the Lck HTRF kinase assay, among others, thereby demonstrating and confirming the utility of the compounds of the invention as protein kinase inhibitors and in the prophylaxis and treatment of immune diseases, hyperproliferative disorders, etc.

LCK-Homogeneous Time Resolved Fluorescent (HTRF) Kinase Assay:

The LCK HTRF assay begins with LCK in the presence of ATP phosphorylating the biotinylated peptide Gastrin. The reaction incubates for 90 min. To quench the assay detection reagents are added which both stop the reaction by diluting out the enzyme and chelating the metals due to the presence of EDTA. Once the detection reagents are added the assay incubates for 30 min to allow for equilibration of the detection reagents.

The LCK HTRF assay is comprised of 10 μL of compound in 100% DMSO, 15 μL of ATP and biotinylated Gastrin, and 15 μL of LCK KD GST (225-509) for a final volume of 40 μL. The final concentration of gastrin is 1.2 μM. The final concentration of ATP is 0.5 μM (Km app=0.6 μM±0.1) and the final concentration of LCK is 250 pM. Buffer conditions are as follows: 50 mM HEPES pH 7.5, 50 mM NaCl, 20 mM MgCl, 5 mM MnCl, 2 mM DTT, 0.05% BSA.

The assay is quenched and stopped with 160 μL of detection reagent. Detection reagents are as follows: Buffer made of 50 mM Tris, pH 7.5, 100 mM NaCl, 3 mM EDTA, 0.05% BSA, 0.1% Tween20. Added to this buffer prior to reading is Steptavidin allophycocyanin (SA-APC) at a final conc in the assay of 0.0004 mg/mL, and europilated anti-phosphotyrosine Ab (Eu-anti-PY) at a final conc of 0.025 nM.

The assay plate is read in either a Discovery or a RubyStar. The eu-anti-PY is excited at 320 nm and emits at 615 nm to excite the SA-APC which in turn emits at 655 nm. The ratio of SA-APC at 655 nm (excited due to close proximity to the Eu-anti-PY because of phosphorylation of the peptide) to free Eu-anti-PY at 615 nm will give substrate phosphorylation.

Assays for other kinases are done in a similar way as described above, varying the concentrations of enzyme, peptide substrate, and ATP added to the reaction, depending on the specific activity of the kinase and measured Km's for the substrates.

Human Mixed Lymphocyte Reaction (huMLR):

The purpose of this assay is to test the potency of T cell activation inhibitors in an in vitro model of allogeneic T cell stimulation. Human peripheral blood lymphocytes (hPBL; 2×10⁵/well) are incubated with mitomycin C-treated B lymphoblastoid cells (JY cell line; 1×10⁵/well) as allogeneic stimulators in the presence or absence of dilutions of potential inhibitor compound in 96-well round-bottom tissue culture plates. These cultures are incubated at 37° C. in 5% CO₂ for 6 days total. The proliferative response of the hPBL is measured by ³H-thymidine incorporation overnight between days 5 and 6 after initiation of culture. Cells are harvested onto glass fiber filters and ³H-thymidine incorporation into DNA is analyzed by liquid scintillation counter.

Jurkat Proliferation/Survival Assay:

The purpose of this assay is to test the general anti-proliferative/cytotoxic effect of compounds on the Jurkat human T cell line. Jurkat cells (1×10⁵/well) are plated in 96-well flat-bottom tissue culture plates with or without compound dilutions and cultured for 72 h at 37° C. in 5% CO₂. Viable cell number is determined during the last 4 h of culture by adding 10 μL/well WST-1 dye. WST-1 dye conversion relies on active mitochondrial electron transport for reduction of the tetrazolium dye. The dye conversion is read by OD at 450-600 nm.

Anti-CD3/CD28-Induced T cell IL-2 Secretion and Proliferation Assay:

The purpose of this assay is to test the potency of T cell receptor (TCR; CD3) and CD28 signaling pathway inhibitors in human T cells. T cells are purified from human peripheral blood lymphocytes (hPBL) and pre-incubated with or without compound prior to stimulation with a combination of an anti-CD3 and an anti-CD28 antibody in 96-well tissue culture plates (1×10⁵ T cells/well). Cells are cultured for ˜20 h at 37° C. in 5% CO₂, then secreted IL-2 in the supernatants is quantified by cytokine ELISA (Pierce/Endogen). The cells remaining in the wells are then pulsed with ³H-thymidine overnight to assess the T cell proliferative response. Cells are harvested onto glass fiber filters and ³H-thymidine incorporation into DNA is analyzed by liquid scintillation counter. For comparison purposes, phorbol myristic acid (PMA) and calcium ionophore can be used in combination to induce IL-2 secretion from purified T cells. Potential inhibitor compounds can be tested for inhibition of this response as described above for anti-CD3 and -CD28 antibodies.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

For the treatment of Lck-mediated diseases and other diseases listed above, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, pain, inflammation and the like.

While it may be possible to administer a compound of the invention alone, in the methods described, the compound administered normally will be present as an active ingredient in a pharmaceutical composition. Thus, in another embodiment of the invention, there is provided a pharmaceutical composition comprising a compound of this invention in combination with a pharmaceutically acceptable carrier, which includes diluents, excipients and the like as described herein. A pharmaceutical composition of the invention may comprise an effective amount of a compound of the invention or an effective dosage amount of a compound of the invention. An effective dosage amount of a compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multidose pharmaceutical composition, such as powders, liquids and the like, in which an effective amount of the compound is administered by administering a portion of the composition.

The dosage regimen for treating Lck-mediated diseases and other diseases listed above with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

1. A compound of Formula I 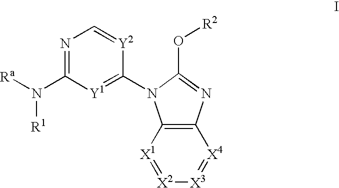
or a pharmaceutically-acceptable salt thereof, wherein
X¹ is N or C(R^{3a}); X² is N or C(R^{3b}); X³ is N or C(R^{3c}); X⁴ is N or C(R^{3d});
Y¹ is N or CH; Y² is N or CH;
R¹ is selected from —R¹¹, —R¹¹—R¹², —R¹¹—R¹⁴, —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴, and —R¹¹—R¹²—R¹³—R¹⁴, any of which is substituted by 0, 1, 2, 3 or 4 substituents independently selected from R^{c};
alternatively R¹ and R^{a} taken together with the nitrogen to which they are attached form a 5- or 6-membered heterocyclic ring having 0, 1 or 2 additional heteroatoms selected from N, O and S, which heterocyclic ring is substituted by 0, 1, 2, 3 or 4 substituents independently selected from R^{c};
R² is selected from —R²¹, —R²¹—R²², —R²¹—R²⁴, —R²²—R²⁴, R²¹—R²²—R²⁴, —R²¹—R²³—R²⁴, —R²²—R²³—R²⁴, —R²¹—R²³—R²²—R²⁴ and —R²¹—R²²—R²³—R²⁴, and of which is substituted by 0, 1, 2, 3 or 4 substituents independently selected from R^{c};
R^{3a} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3a} is independently in each instance selected from R^{c};
R^{3b} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3b} is independently in each instance selected from R^{c};
R^{3c} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3c} is independently in each instance selected from R^{c};
R^{3d} is independently in each instance, selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3d} is independently in each instance selected from R^{c};
R¹¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R¹² is independently at each instance C₁₋₈alkyl;
R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R¹⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²² is independently at each instance C₁₋₈alkyl;
R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R²⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³² is independently at each instance C₁₋₈alkyl;
R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R³⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R^{a} is independently at each instance H or R^{b};
R^{b} is independently at each instance C₁₋₈alkyl, phenyl or benzyl; and
R^{c} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

2. The compound of claim 1 wherein one of X¹, X², X³ and X⁴ is N and the other three of X¹, X², X³ and X⁴ is C(R^{3a}), C(R^{3b}), C(R^{3c}), or C(R^{3d}).

3. The compound of claim 1 wherein Y¹ is N and Y² is CH.

4. The compound of claim 1 wherein Y¹ is N and Y² is N.

5. The compound of claim 1 wherein X¹ is C(R^{3a}); X² is C(R^{3b}); X³ is C(R^{3c}); and X⁴ is C(R^{3d}).

6. The compound of claim 1, defined by formula II 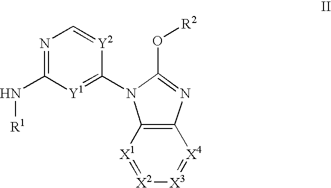
wherein X¹ is N or C(R^{3a}); X² is N or C(R^{3b}); X³ is N or C(R^{3c}); X⁴ is N or C(R^{3d});
Y¹ is N or CH; Y² is N or CH;
R¹ is selected from —R¹¹, —R¹¹—R¹², —R¹¹—R¹⁴, —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴ and —R¹¹—R¹²—R¹³—R¹⁴, and of which is substituted by 0, 1, 2, 3 or 4 substituents independently selected from R^{c};
R² is selected from —R²¹, —R²¹—R²², —R²¹—R²⁴, —R²²—R²⁴, —R²¹—R²²—R²⁴, —R²¹—R²³—R²⁴, —R²²—R²³—R²⁴, —R²¹—R²³—R²²—R²⁴ and —R²¹—R²²—R²³—R²⁴, any of which is substituted by 0, 1, 2, 3 or 4 substituents independently selected from R^{c};
R^{3a} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3a} is independently in each instance selected from R^{c};
R^{3b} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3b} is independently in each instance selected from R^{c};
R^{3c} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3c} is independently in each instance selected from R^{c};
R^{3d} is independently in each instance, selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3d} is independently in each instance selected from R^{c};
R¹¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R¹² is independently at each instance C₁₋₈alkyl;
R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R¹⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R²² is independently at each instance C₁₋₈alkyl;
R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R²⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³¹ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R³² is independently at each instance C₁₋₈alkyl;
R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R³⁴ is independently at each instance a saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, so long as the combination of O and S atoms is not greater than 2, wherein the carbon atoms of the ring are substituted by 0, 1 or 2 oxo groups;
R^{a} is independently at each instance H or R^{b};
R^{b} is independently at each instance C₁₋₈alkyl, phenyl or benzyl; and
R^{c} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

7. The compound of claim 6, wherein
X¹ is C(R^{3a}); X² is C(R^{3b}); X³ is C(R^{3c}); X⁴ is C(R^{3d});
Y¹ is N; Y² is N or CH;
R¹ is selected from —R¹¹, —R¹¹—R¹², —R¹¹—R¹⁴, —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴ and —R¹¹—R¹²—R¹³—R¹⁴, and of which is substituted by 0, 1, 2, 3 or 4 substituents independently selected from R^{c};
R² is selected from —R²¹, —R²¹—R²², —R²¹—R²⁴, —R²²—R²⁴, —R²¹—R²²—R²⁴, —R²¹—R²³—R²⁴, —R²²—R²³—R²⁴, —R²¹—R²³—R²²—R²⁴ and —R²¹—R²²—R²³—R²⁴, any of which is substituted by 0, 1, 2, 3 or 4 substituents independently selected from R^{c};
R^{3a} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3a} is independently in each instance selected from R^{c};
R^{3b} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3b} is independently in each instance selected from R^{c};
R^{3c} is selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3c} is independently in each instance selected from R^{c};
R^{3d} is independently in each instance, selected from H, —R³², —R³⁴, —R³²—R³⁴, —R³³—R³⁴, —R³³—R³²—R³⁴ and —R³²—R³³—R³⁴, any of which is substituted by 0, 1, 2, 3 or 4 substituents independently selected from R^{c}; or R^{3d} is independently in each instance selected from R^{c};
R¹¹ is independently at each instance a phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups;
R¹² is independently at each instance C₁₋₈alkyl;
R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R¹⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;
R²¹ is independently at each instance a phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups;
R²² is independently at each instance C₁₋₈alkyl;
R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R²⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;
R³¹ is independently at each instance a phenyl, naphthyl, pyridyl, pyrimidinyl, triazinyl, quinolinyl, isoquinolinyl, quinazolinyl, isoquinazolinyl, thiophenyl, furyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, isoindolyl, indazolyl, benzofuranyl, benzothiophenyl, benzimidazolyl, imidazo-pyridinyl, purinyl, benzotriazolyl, oxazolinyl, isoxazolinyl or thiazolinyl ring, wherein the carbon atoms of the ring are substituted by 0, 1 or 2 oxo groups;
R³² is independently at each instance C₁₋₈alkyl;
R³³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R³⁴ is independently at each instance a phenyl, naphthyl, 5,6,7,8-tetrahydronaphthyl, dihydro-indenyl, pyridyl, pyrimidinyl, triazinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;
R^{a} is independently at each instance H or R^{b};
R^{b} is independently at each instance C₁₋₈alkyl, phenyl or benzyl; and
R^{c} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

8. The compound of claim 7 wherein
X¹ is C(R^{3a}); X² is C(R^{3b}); X³ is C(R^{3c}); X⁴ is C(R^{3d});
Y¹ is N; Y² is CH;
R¹ is selected from —R¹¹, —R¹¹—R¹², —R¹¹—R¹⁴, —R¹²—R¹⁴, —R¹¹—R¹²—R¹⁴, —R¹¹—R¹³—R¹⁴, —R¹²—R¹³—R¹⁴, —R¹¹—R¹³—R¹²—R¹⁴ and —R¹¹—R¹²—R¹³—R¹⁴, and of which is substituted by 0, 1, 2, 3 or 4 substituents independently selected from R^{c};
R² is selected from —R²¹, —R²¹—R²², —R²¹—R²⁴, —R²²—R²⁴, —R²¹—R²²—R²⁴, —R²¹—R²³—R²⁴, —R²²—R²³—R²⁴, —R²¹—R²³—R²²—R²⁴ and —R²¹—R²²—R²³—R²⁴, any of which is substituted by 0, 1, 2, 3 or 4 substituents independently selected from R^{c};
R^{3a} is selected from R^{a}, R^{b} and R^{c};
R^{3b} is selected from R^{a}, R^{b} and R^{c};
R^{3c} is selected from R^{a}, R^{b} and R^{c};
R^{3d} is selected from R^{a}, R^{b} and R^{c};
R¹¹ is independently at each instance a phenyl, pyridyl, pyrimidinyl, thiophenyl, thiazolyl, oxazolyl, isoxazolyl, isothiazolyl, oxazolinyl, isoxazolinyl or thiazolinyl ring;
R¹² is independently at each instance C₁₋₈alkyl;
R¹³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R¹⁴ is independently at each instance a phenyl, pyridyl, pyrimidinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;
R²¹ is independently at each instance a phenyl, pyridyl, pyrimidinyl, thiophenyl, thiazolyl, oxazolyl, isoxazolyl, isothiazolyl, oxazolinyl, isoxazolinyl or thiazolinyl ring;
R²² is independently at each instance C₁₋₈alkyl;
R²³ is independently at each instance —C(═O)—, —C(═O)O—, —C(═O)NR^{a}—, —C(═NR^{a})NR^{a}—, —O—, —OC(═O)—, —OC(═O)NR^{a}—, —OC(═O)N(R^{a})S(═O)₂—, —OC₂₋₆alkylNR^{a}—, —OC₂₋₆alkylO—, —S—, —S(═O)—, —S(═O)₂—, —S(═O)₂NR^{a}—, —S(═O)₂N(R^{a})C(═O)—, —S(═O)₂N(R^{a})C(═O)O—, —S(═O)₂N(R^{a})C(═O)NR^{a}—, —N(R^{a})—, —N(R^{a})C(═O)—, —N(R^{a})C(═O)O—, —N(R^{a})C(═O)N(R^{a})—, —N(R^{a})C(═NR^{a})N(R^{a})—, —N(R^{a})S(═O)₂—, —N(R^{a})S(═O)₂N(R^{a})—, —NR^{a}C₂₋₆alkylN(R^{a})— or —NR^{a}C₂₋₆alkylO—;
R²⁴ is independently at each instance a phenyl, pyridyl, pyrimidinyl, quinolinyl, tetrahydroquinolinyl, isoquinolinyl, tetrahydroisoquinolinyl, quinazolinyl, isoquinazolinyl, thiophenyl, furyl, tetrahydrofuranyl, pyrrolyl, pyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, cyclopropyl, cyclobutyl, azetidinyl, cyclopentyl, cyclohexyl or cycloheptyl ring, wherein the carbon atoms of the ring are substituted by 0, 1 or 2 oxo groups;
R^{a} is independently at each instance H or R^{b};
R^{b} is independently at each instance C₁₋₈alkyl, phenyl or benzyl; and
R^{c} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

9. A compound according to claim 1, wherein the compound is selected from:
4-(2-((2,6-dimethylphenyl)oxy)-1H-benzimidazol-1-yl)-N-(4-(4-methyl-1-piperazinyl)phenyl)-2-pyrimidinamine;
4-(2-((2-(methyloxy)phenyl)oxy)-1H-benzimidazol-1-yl)-N-(4-(4-morpholinyl)phenyl)-2-pyrimidinamine;
4-(2-((2-(methyloxy)phenyl)oxy)-1H-benzimidazol-1-yl)-N-(4-(4-methyl-1-piperazinyl)phenyl)-2-pyrimidinamine;
4-(4-(4-(2-((2-(methyloxy)phenyl)oxy)-1H-benzimidazol-1-yl)-2-pyrimidinyl)-1-piperazinyl)phenylamine;
N-(4-((2-((1-methylethyl)amino)ethyl)oxy)-3-(methyloxy)phenyl)-4-(2-((2-(methyloxy)phenyl)oxy)-1H-benzimidazol-1-yl)-2-pyrimidinamine;
4-(2-((2-(methyloxy)phenyl)oxy)-1H-benzimidazol-1-yl)-N-(3-(methyloxy)-4-(((2S)-2-pyrrolidinylmethyl)oxy)phenyl)-2-pyrimidinamine;
4-(2-((2-(methyloxy)phenyl)oxy)-1H-benzimidazol-1-yl)-N-(3-(methyloxy)-4-(((2R)-2-pyrrolidinylmethyl)oxy)phenyl)-2-pyrimidinamine;
N-(3-chloro-4-((2-((1-methylethyl)amino)ethyl)oxy)phenyl)-4-(2-((2-(methyloxy)phenyl)oxy)-1H-benzimidazol-1-yl)-2-pyrimidinamine;
N-(4-((2-((1-methylethyl)amino)ethyl)oxy)phenyl)-4-(2-((2-(methyloxy)phenyl)oxy)-1H-benzimidazol-1-yl)-2-pyrimidinamine;
N-(4-((2-(dimethylamino)ethyl)oxy)phenyl)-4-(2-((2-(methyloxy)phenyl)oxy)-1H-benzimidazol-1-yl)-2-pyrimidinamine;
4-(2-((2,3-bis(methyloxy)phenyl)oxy)-1H-benzimidazol-1-yl)-N-(3-chloro-4-((2-((1-methylethyl)amino)ethyl)oxy)phenyl)-2-pyrimidinamine;
4-(2-((2,3-bis(methyloxy)phenyl)oxy)-1H-benzimidazol-1-yl)-N-(4-((2-((1-methylethyl)amino)ethyl)oxy)phenyl)-2-pyrimidinamine;
4-(2-(pyridine-2-ylmethoxy)-1H-benzo[d]imidazol-1-yl)-N-3,4,5-trimethoxyphenyl)-1,3,5-triazin-2-amine;
or a pharmaceutically-acceptable salt thereof.

10. A method for making a compound according to claim 6, comprising the steps of:
reacting a compound having the structure 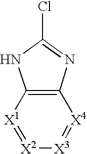
with dialkylcarbonate to give 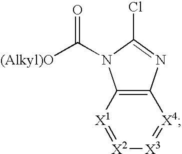
reacting the product with R²OH to give 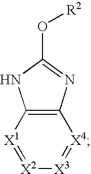
reacting the formed product with 2,4-dihalopyrimidine to give 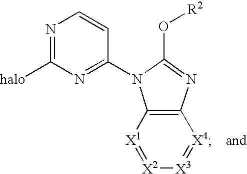
reacting the halopyrimidine with H₂N—R¹ in the presence of acid to give

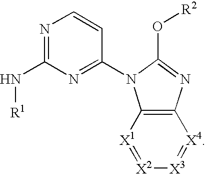

11. A pharmaceutical composition comprising a compound according to claim 1 and a pharmaceutically acceptable carrier.

12. A pharmaceutical composition comprising a compound according to claim 6 and a pharmaceutically acceptable carrier.

13. A method of treatment of inflammation, the method comprising the step of administering a therapeutically-effective amount of a compound according to claim 1.

14. A method of treatment of inflammation, the method comprising the step of administering a therapeutically-effective amount of a compound according to claim 6.

15. A method of inhibition of T cell activation and proliferation in a mammal, the method comprising the step of administering an therapeutically-effective amount of a compound according to claim 1.

16. A method of inhibition of T cell activation and proliferation in a mammal, the method comprising the step of administering an therapeutically-effective amount of a compound according to claim 6.

17. A method of treatment of arthritis, rheumatoid arthritis, psoriatic arthritis, or osteoarthritis in a mammal, the method comprising administering a therapeutically-effective amount of a compound according to claim 1.

18. A method of treatment of arthritis, rheumatoid arthritis, psoriatic arthritis, or osteoarthritis in a mammal, the method comprising administering a therapeutically-effective amount of a compound according to claim 6.

19. A method of treatment of organ transplant, acute transplant or heterograft or homograft rejection, or transplantation tolerance induction in a mammal, the method comprising administering a therapeutically-effective amount of a compound according to claim 1.

20. A method of treatment of organ transplant, acute transplant or heterograft or homograft rejection, or transplantation tolerance induction in a mammal, the method comprising administering a therapeutically-effective amount of a compound according to claim 6.

21. A method of treatment of ischemic or reperfusion injury, myocardial infarction, or stroke in a mammal, the method comprising administering a therapeutically-effective amount of a compound according to claim 1.

22. A method of treatment of ischemic or reperfusion injury, myocardial infarction, or stroke in a mammal, the method comprising administering a therapeutically-effective amount of a compound according to claim 6.

23. A method of treatment of multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea or atopic dermatitis in a mammal, the method comprising administering a therapeutically-effective amount of a compound according to claim 1.

24. A method of treatment of multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea or atopic dermatitis in a mammal, the method comprising administering a therapeutically-effective amount of a compound according to claim 6.

25. A method of treatment of colon carcinoma or thymoma in a mammal, the method comprising administering a therapeutically-effective amount of a compound according to claim 1.

26. A method of treatment of colon carcinoma or thymoma in a mammal, the method comprising administering a therapeutically-effective amount of a compound according to claim 6.

27. The manufacture of a medicament comprising a compound according to claim 1.

28. The manufacture of a medicament comprising a compound according to claim 6.

29. The manufacture of a medicament for the treatment of inflammation comprising a therapeutically-effective amount of a compound according to claim 1.

30. The manufacture of a medicament for the treatment of inflammation comprising a therapeutically-effective amount of a compound according to claim 6.

31. The manufacture of a medicament for the inhibition of T cell activation and proliferation in a mammal in need thereof, comprising a therapeutically-effective amount of a compound according to claim 1.

32. The manufacture of a medicament for the inhibition of T cell activation and proliferation in a mammal in need thereof, comprising a therapeutically-effective amount of a compound according to claim 6.

33. The manufacture of a medicament for the treatment of arthritis, rheumatoid arthritis, psoriatic arthritis, or osteoarthritis in a mammal comprising a therapeutically-effective amount of a compound according to claim 1.

34. The manufacture of a medicament for the treatment of arthritis, rheumatoid arthritis, psoriatic arthritis, or osteoarthritis in a mammal comprising a therapeutically-effective amount of a compound according to claim 6.

35. The manufacture of a medicament for the treatment of organ transplant, acute transplant or heterograft or homograft rejection, or transplantation tolerance induction in a mammal comprising a therapeutically-effective amount of a compound according to claim 1.

36. The manufacture of a medicament for the treatment of organ transplant, acute transplant or heterograft or homograft rejection, or transplantation tolerance induction in a mammal comprising a therapeutically-effective amount of a compound according to claim 6.

37. The manufacture of a medicament for the treatment of ischemic or reperfusion injury, myocardial infarction, or stroke in a mammal in need thereof, comprising a therapeutically-effective amount of a compound according to claim 1.

38. The manufacture of a medicament for the treatment of ischemic or reperfusion injury, myocardial infarction, or stroke in a mammal in need thereof, comprising a therapeutically-effective amount of a compound according to claim 6.

39. The manufacture of a medicament for the treatment of multiple sclerosis, inflammatory bowel disease, including ulcerative colitis, Crohn's disease, lupus, contact hypersensitivity, delayed-type hypersensitivity, and gluten-sensitive enteropathy, type 1 diabetes, psoriasis, contact dermatitis, Hashimoto's thyroiditis, Sjogren's syndrome, autoimmune hyperthyroidism, Addison's disease, autoimmune polyglandular disease, autoimmune alopecia, pernicious anemia, vitiligo, autoimmune hypopituatarism, Guillain-Barre syndrome, glomerulonephritis, serum sickness, uticaria, allergic diseases, asthma, hayfever, allergic rhinitis, scleracielma, mycosis fungoides, dermatomyositis, alopecia areata, chronic actinic dermatitis, eczema, Behcet's disease, Pustulosis palmoplanteris, Pyoderma gangrenum, Sezary's syndrome, atopic dermatitis, systemic schlerosis, morphea or atopic dermatitis in a mammal comprising a therapeutically-effective amount of a compound according to claim 6.

40. The manufacture of a medicament for the treatment of colon carcinoma or thymoma in a mammal comprising a therapeutically-effective amount of a compound according to claim 6.
